(19) United States
(12) Patent Application Publication
(10) Pub. No.: US 2026/0365611 A1
(43) Pub. Date: Nov. 19, 2026

(54) CRYSTALLINE SALTS OF 2-PHENYL-2-AMINOCYCLOHEXAN-1-ONE DERIVATIVES

(71) Applicant: Gilgamesh Pharma Inc., New York, NY (US)

(72) Inventors: William LEONG, New York, NY (US); Yow-Rong Catrina JOU, New York, NY (US); Kaushalendra CHATURVEDI, New York, NY (US); Qi GAO, New York, NY (US)

(73) Assignee: Gilgamesh Pharma Inc., New York, NY (US)

(21) Appl. No.: 19/490,052

(22) PCT Filed: Jun. 5, 2024
(86) PCT No.: PCT/US2024/032487

§ 371 Date: Dec. 4, 2025

Related U.S. Application Data
(60) Provisional application No. 63/471,091, filed on Jun. 5, 2023.
(60) Provisional application No. 63/580,073, filed on Sep. 1, 2023.

(51) Int. Cl. C07C 225/20

(52) U.S. Cl. CPC C07C 225/20; C07B 2200/13; C07C 2601/14

(57) ABSTRACT

The present disclosure relates to various crystalline salts of (R)-2-(4-fluorophenyl)-2-(methylamino)cyclohexan-1-one, which are useful for treating psychiatric disorders, including treating mood disorders. These salts include the hydrochloride salts, sulfate salts, p-toluenesulfonate salts, methanesulfonate salts, maleate salts, phosphate salts, tartrate salts, fumarate salts, citrate salts, glycolate salts, malate salts, and hippurate salts thereof.

### FIELD OF THE DISCLOSURE

The present disclosure relates to crystalline salts and polymorphs of 2-phenyl-2-aminocyclohexan-1-one derivatives, which are useful in the treatment of psychiatric disorders.

### BACKGROUND OF THE DISCLOSURE

Depression is a common psychological problem and refers to a mental state of low mood and aversion to activity. Various symptoms associated with depression include persistent anxious or sad feelings, feelings of helplessness, hopelessness, pessimism, and/or worthlessness, low energy, restlessness, irritability, fatigue, loss of interest in pleasurable activities or hobbies, excessive sleeping, overeating, appetite loss, Insomnia, thoughts of suicide, and suicide attempts. The presence, severity, frequency, and duration of the above-mentioned symptoms vary on a case-by-case basis.

Approximately one third of patients with major depressive disorder (MDD) fail to achieve remission of their symptoms, even after multiple rounds of treatment with several known classes of antidepressants, including selective serotonin reuptake inhibitors (SSRIs) (Rush et al. 2006). This high prevalence of treatment-resistant depression (TRD) makes clear the need for new, more efficacious pharmacotherapies for depression that will target new mechanisms and/or patient populations.

U.S. Pat. No. 11,344,510, the contents of which are incorporated by reference, discloses, in part, compounds useful for the treatment of psychiatric disorders, including 2-(4-fluorophenyl)-2-(methylamino)cyclohexane-1-one,

US Patent Application having Serial Number U.S. Ser. No. 63/471,091 (SSMP Docket: 41715) entitled “SYNTHESIS OF 2-PHENYL-2-AMINOCYCLOHEXAN-1-ONE DERIVATIVES”, the contents of which are incorporated by reference, discloses, in part, polymorphs of the hydrochloride salt of 2-(4-fluorophenyl)-2-(methylamino)cyclohexan-1-one.

Many compounds may exist in more than one salt form and those salts in turn may each exist in more than one crystal form, or polymorph. Relative to one another, these salts and polymorphs exhibit different physical, chemical, and spectroscopic properties. For example, certain salts or polymorphs of a compound may be more chemically stable, may be more readily crystallized, may be more readily soluble in particular solvents, may be more or less hygroscopic, may flow more readily, or may compress more easily than others. See, e.g., P. DiMartino, et al., J. Thermal Anal., 48:447-458 (1997). In the case of drugs, certain solid forms may be more bioavailable than others, while others may be more stable under certain manufacturing, storage, and biological conditions. This is particularly important from a regulatory standpoint, since drugs are approved by governmental agencies, such as the U.S. Food and Drug Administration, only if they meet exacting purity and characterization standards. Indeed, the regulatory approval of one salt or polymorph of a compound, which exhibits certain solubility and physicochemical (including spectroscopic) properties, does not necessarily imply the ready approval of other salts or polymorphs of that same compound. Salt and polymorphic forms of a compound are known in the pharmaceutical arts to affect, for example, the solubility, stability, flowability, fractability, and compressibility of the compound, as well as the safety and efficacy of drug products comprising it. See, e.g., Knapman, K. Modern Drug Discoveries, 2000, 53. Therefore, the discovery of new salts and polymorphs of a drug can provide a variety of advantages.

However, finding the appropriate conditions for preparing and crystallizing a new salt or polymorph of a particular compound is tedious and often involves trial and error, as many factors come into play to find the right conditions for synthesis and crystallization thereof. For example, solvent, concentration, temperature, heating or cooling rate, stoichiometry of reactants, addition rates, and mixing parameters must all be varied to obtain the desired result.

This present disclosure encompasses polymorphs of certain salts of 2-(4-fluorophenyl)-2-(methylamino)cyclohexan-1-one and stereoisomers thereof, such as the R isomer. Further, as described hereinbelow, it has now been discovered that certain salts of 2-(4-fluorophenyl)-2-(methylamino)cyclohexan-1-one and stereoisomers thereof, such as salts of (R)-2-(4-fluorophenyl)-2-(methylamino)cyclohexan-1-one, can be prepared and isolated in a number of crystal forms.

### SUMMARY OF THE DISCLOSURE

The present disclosure relates to various crystalline salts of (R)-2-(4-fluorophenyl)-2-(methylamino)cyclohexan-1-one, which are useful for treating psychiatric disorders, including treating mood disorders. These salts include the hydrochloride salts, sulfate salts, p-toluenesulfonate salts, methanesulfonate salts, maleate salts, phosphate salts, tartrate salts, fumarate salts, citrate salts, glycolate salts, malate salts, and hippurate salts. In addition, the present disclosure relates to polymorphs of the aforementioned salts in crystalline form.

### BRIEF DESCRIPTION OF THE DRAWINGS

The objects, features, and advantages of the present disclosure will become apparent to one of ordinary skill in the art, in view of the following detailed description, taken in combination with the attached drawings, in which:

FIG. 1 is an ORTEP drawing of the single crystal X-ray structure of 1R HCl in Form 1 (a) and Form 2 (b).

FIG. 2 is an exemplary XRPD diffractogram of solid 1R HCl (Form 1).

FIG. 3 is an exemplary PLM image of solid 1R HCl (Form 1).

FIG. 4 is an exemplary DSC/TGA overlay of solid 1R HCl (Form 1).

FIG. 5 is an exemplary DVS isotherm plot of solid 1R HCl (Form 1) (a) and DVS change in Mass Plot (b).

FIG. 6 is an exemplary XPRD of solid 1R HCl as a mixture of Form 1 and Form 2.

FIG. 7 is a simulated XRPD of solid 1R HCl (Form 2) generated from single crystal data.

FIG. 8 is an exemplary XRPD diffractogram of Sulfate-1.

FIG. 9 is an exemplary DSC/TGA overlay of Sulfate-1.

FIG. 10 is an exemplary XRPD diffractogram of Sulfate-2.

FIG. 11 is an exemplary XRPD diffractogram of Sulfate-3.

FIG. 12 is an exemplary XRPD diffractogram of Sulfate-4.

FIG. 13 is an exemplary XRPD diffractogram of Tosyl-1.

FIG. 14 is an exemplary DSC/TGA overlay of Tosyl-1.

FIG. 15 is an exemplary XRPD diffractogram of Tosyl-2A.

FIG. 16 is an exemplary XRPD diffractogram of Tosyl-2B.

FIG. 17 is an exemplary DSC/TGA overlay of Tosyl-2B.

FIG. 18 is an exemplary XRPD diffractogram of MSA-1.

FIG. 19 is an exemplary DSC/TGA overlay of MSA-1.

FIG. 20 is an exemplary XRPD diffractogram of Mal-1.

FIG. 21 is an exemplary DSC/TGA overlay of Mal-1.

FIG. 22 is an exemplary XRPD diffractogram of Mal-2.

FIG. 23 is an exemplary DSC/TGA overlay of Mal-2.

FIG. 24 is an exemplary XRPD diffractogram of Phos-1.

FIG. 25 is an exemplary DSC/TGA overlay of Phos-1.

FIG. 26 is an exemplary XRPD diffractogram of Phos-2.

FIG. 27 is an exemplary DSC/TGA overlay of Phos-2.

FIG. 28 is an exemplary XRPD diffractogram of Tar-1.

FIG. 29 is an exemplary XRPD diffractogram of Tar-2.

FIG. 30 is an exemplary XRPD diffractogram of Tar-5.

FIG. 31 is an exemplary DSC/TGA overlay of Tar-5.

FIG. 32 is an exemplary XRPD diffractogram of Tar-3

FIG. 33. is an exemplary DSC/TGA overlay of Tar-3.

FIG. 34 is an exemplary XRPD diffractogram of Tar-4.

FIG. 35 is an exemplary XRPD diffractogram of Tar-6

FIG. 36 is an exemplary DSC/TGA overlay of Tar-6.

FIG. 37 is an exemplary XRPD diffractogram of Fum-1.

FIG. 38 is an exemplary DSC/TGA overlay of Fum-1.

FIG. 39 is an exemplary XRPD diffractogram of Fum-2.

FIG. 40 is an exemplary XRPD diffractogram of Cit-1.

FIG. 41 is an exemplary XRPD diffractogram of Cit-2.

FIG. 42 is an exemplary DSC/TGA overlay of Cit-2.

FIG. 43 is an exemplary XRPD diffractogram of Glyc-1.

FIG. 44 is an exemplary DSC/TGA overlay of Glyc-1.

FIG. 45 is an exemplary XRPD diffractogram of Mali-1.

FIG. 46 is an exemplary DSC/TGA overlay of Mali-1.

FIG. 47 is an exemplary XRPD diffractogram of Hip-1

FIG. 48. is an exemplary DSC/TGA overlay of Hip-1.

### DETAILED DESCRIPTION OF THE DISCLOSURE

The salts disclosed herein include at least one asymmetric center. When the stereoisomers are specifically designated, these centers are specifically indicated by the symbols “R” or “S,” depending on the configuration of substituents around the chiral atom. However, when stereochemistry is not to be designated, the structures will be drawn without indicating the stereochemistry; these structures are racemic mixtures. “R,” as used herein, whether bolded or not bolded, italicized or not italicized, refers to the R isomer, while the term “S,” as used herein, whether bolded or not bolded, italicized or not italicized, refers to the S isomer.

In some embodiments, a composition prepared herein may be enriched in a specific enantiomer of any compound disclosed herein relative to the corresponding opposite enantiomer of that compound, such that the mixture is not racemic. In such cases, the subject mixture of isomers is understood to have an enantiomeric excess and optical purity >0%. The enantiomeric excess or optical purity of the isomeric mixture may be, for example, >0%, >5%, >25%, >50%, >75%, >90%, >95%, >97%, >98%, or >99%. The enantiomeric excess or optical purity of the isomeric mixture may be, for example, 5-100%, 25-100%, 50-100%, 75-100%, 90-100%, 95-100%, 97-100%, 98-100%, or 99-100%. Thus, for example, contemplated herein is a composition including the S enantiomer of a compound substantially free of the R enantiomer, or the R enantiomer substantially free of the S enantiomer. Further, if the named compound includes more than one chiral center, the scope of the present disclosure also includes compositions containing the various stereoisomers and diastereomers, including mixtures of varying proportions between the various stereoisomers and/or diastereomers or pharmaceutically acceptable salts thereof, as well as compositions including one or more stereoisomers and diastereomers substantially free of one or more of the other stereoisomers and/or diastereomers, respectively. By “substantially free” in this context, it is meant that the composition includes less than, for example, 50%, 25%, 15%, 10%, 8%, 5%, 3%, 2%, or 1% of the minor enantiomer or diastereomer(s). For example, the expression that a compound is enantiomerically pure refers to the compound being substantially free of other stereoisomers, including any other enantiomers or diastereomers.

For clarity, in the context of the present disclosure, chemical structures of a compound depicted with a specific stereochemical orientation at any particular chiral center, as defined by wedge and dash notation, are intended to represent the specified stereoisomer of said compound in substantially pure form, or a mixture enriched in the stereoisomer(s) with the specified stereochemical orientation at the defined chiral center over the stereoisomer(s) with the opposite orientation at said chiral center.

As used herein, the term that the “composition is substantially free,” as relates to a polymorph, refers to the composition being predominantly present in a particular polymorph relative to the other polymorph. For example, 1R HCl exists in two solid forms, Form 1 and Form 2. The statement that 1R HCl exists in Form 1 substantially free from Form 2 means that the Form 1 polymorph is the predominant polymorph present relative to Form 2, i.e., the Form 1 polymorph is present in excess relative to the Form 2 polymorph. By “substantially free” of Form 2 relative to Form 1, it is meant that the composition includes less than, for example, 50%, 25%, 15%, 10%, 8%, 5%, 3%, 2%, or 1% of the Form 2 polymorph relative to the Form 1 polymorph. Similarly, using the term “the composition is substantially free” of Form 1 relative to Form 2 is meant to indicate that the composition includes less than, for example, 50%, 25%, 15%, 10%, 8%, 5%, 3%, 2%, or 1% of the Form 1 polymorph relative to the Form 2 polymorph.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range of up to 10%, a range of up to 5%, and/or a range of up to 1% of a given value. Alternatively, particularly with respect to biological systems or processes, the term can mean within an order of magnitude, e.g., within 5-fold, or within 2-fold, of a value. “About” and “approximately” are used interchangeably herein.

As defined herein, an “inert solvent” is a solvent that does not react with either the reactants or products formed in the reaction. Suitable organic solvents for use in the present disclosure include, but are not limited to, alcohols having 1-6 carbon atoms, such as methanol, ethanol, isopropanol, butanol and the like; ketones having 1-6 carbon atoms, such as acetone, methyl ethyl ketone, methyl isobutyl ketone, and the like; ether solvents having 1-6 carbon atoms, such as dimethyl ether, diethyl ether, methyl ethyl ether, methyl t-butyl ether MTBE, dipropyl ether, diisopropyl ether and the like, cyclic ethers having 4-6 carbon atoms, such as THF, dioxane, and the like; halogenated solvents such as dichloroethane, dichloromethane, chloroform and the like; esters having 2-10 carbon atoms, such as ethyl acetate, isopropyl acetate, n-propyl acetate and the like; nitriles such as acetonitrile, propionitrile and the like; hydrocarbons having 1-10 carbon atoms, including aryl groups, such as toluene, xylene, cyclohexane, heptane, xylene and the like; dimethylsulfoxide (DMSO), N,N-dimethylformamide (DMF), N,N-dimethylacetamide (DMA) and the like; and mixtures thereof in various proportion without limitation. The use of a suitable solvent includes the use of a mixture of solvents. The applicability of a particular solvent for a reaction is dependent on several factors, such as the type of reaction, the reactants, the products, reagents used, and the like. One of ordinary skill in the art can determine the appropriate solvents for the reactions described herein.

As used herein, the term “protic” refers to a proton or a hydrogen atom or ion. The term “protic solvent,” as used herein, refers to a polar liquid compound that has dissociable hydrogen atoms and is capable of forming a hydrogen bond with oxygen, fluorine, or nitrogen atoms.

The term “protic polar solvent,” as used herein, refers to a solvent that has at least one OH or NH bond and is miscible with water. Examples include water, methanol, ethanol, ammonia, and the like.

XRPD patterns of polymorphs are depicted herein. Each polymorph is characterized by or has an XPRD pattern substantially as shown in its corresponding aforementioned figure, e.g., at least 70% of the XPRD pattern has the peaks at the values shown with variations of ±0.50 ° 2θ, and in another embodiment, at least 75% of the XPRD pattern has the peaks at the values shown with variations of ±0.50 ° 2θ, and in another embodiment, at least 80% of the XPRD pattern has the peaks at the values shown with variations of ±0.50 ° 2θ, and in a still further embodiment, at least 85% of the XPRD pattern has the peaks at the values shown with variations of ±0.50 ° 2θ, and in still another embodiment, at least 90% of the XPRD pattern has the peaks at the values shown with variations of ±0.50 ° 2θ. It should be understood, however, that relative intensities and assignment of the peaks of polymorphic forms depicted in these figures can vary depending on a number of factors, including, without limitation, sample preparation, aspect ratio, particle size, mounting, and the instrument and analytical procedure and settings used to obtain the spectrum. As such, the peaks observed in the figures and assignments listed herein in the various tables and figures are intended to encompass variations of ±0.5 ° 2θ and variations in relative peak intensity understood to be acceptable by one skilled in the art. However, it is to be understood that peak values in the tables and figures also encompass variations of ±0.1, ±0.2, ±0.3, and ±0.4 ° 2θ, or any value therein between. When listing the peaks for the XRPDs, it is to be understood that each of the values listed are ±0.50 ° 2θ, even when ±0.50 ° 2θ is not recited in the listing. Further, when a listing of peaks is provided with a variation at the end of the list, e.g., ±0.50 ° 2θ, for purposes of this disclosure, each value in the list of peaks is modified by the listed variation, e.g. ±0.50 ° 2θ. In other embodiments, the depicted or listed XRPD peaks have variations of between ±1 ° 2θ and ±0 ° 2θ, e.g., ±1 ° 2θ, ±0.75 ° 2θ, ±0.20 ° 2θ, or ±0.10 ° 2θ.

Unless indicated to the contrary, the term “freebase” or “free base” refers to 2-(4-fluorophenyl)-2-(methylamino)cyclohexan-1-one in its base, unprotonated form without any counterion, or one of its enantiomers, if indicated, e.g., R freebase or S freebase. Thus, as used herein, the term “freebase” refers to the structure

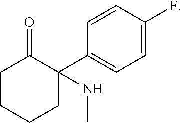 [SMILES: CNC1(c2ccc(F)cc2)CCCCC1=O]

Therefore, the “R freebase” refers to the R isomer of this structure and the “S freebase” refers to the S isomer of this structure. Further, the term “deuterated freebase” refers to the structure

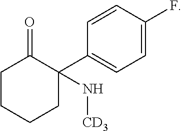 [SMILES: [2H]C([2H])([2H])NC1(c2ccc(F)cc2)CCCCC1=O]

Therefore, the “R deuterated freebase” refers to the R isomer of this structure and the “S deuterated freebase” refers to the S isomer of this structure.

As defined herein, the term “solvent” refers to a liquid substance or a mixture of liquid substances, which is capable of dissolving another substance (solute) to form a solution in which the solute is uniformly dispersed at the molecular or ionic size level. For purposes of this disclosure, the solvent may be a reaction solvent or a crystallizing solvent. Solvents referenced herein are inert solvents with respect to the solutes or reactants.

As defined herein, an “inert solvent” is a solvent that does not react with either the reactants or products formed in a chemical reaction.

The term “reaction solvent” or like term is a solvent in which a chemical reaction occurs. It is an inert solvent, i.e., it does not react with either the free base or the acids or the product that is formed. In an embodiment, the free base and the acid are soluble therein, and the product salt may or may not be soluble therein. Further, in an embodiment, it is a volatile solvent. Further, in an embodiment, it is a volatile solvent, which has a boiling point of 90 °C or less at 1 atm pressure.

As used herein, the term “crystallizing solvent” or like term is an inert solvent that is used for the crystallization of a salt of the present disclosure in which the salt is poorly soluble at room temperature or low temperature, but in which it is more soluble when heated, for example, to the boiling point of the solvent. Ideally, the salt is nearly insoluble or sparingly soluble in the solvent at room temperature and extremely soluble at the boiling point of the solvent. The crystallizing solvent may be one solvent or a mixture of solvents. If it is a mixture of liquid solvents, they may be miscible. In an embodiment, water may be a solvent or co-solvent. The term “recrystallizing solvent,” as used herein, is a crystallizing solvent, and the two terms may be used interchangeably.

The term “crystalline” as applied to a compound refers to a solid phase in which the material has a regularly ordered internal structure at the molecular level and gives a distinctive X-ray diffraction pattern with defined peaks. By substantially crystalline, it is meant that the composition has greater than 50%, or greater than 55%, or greater than 60%, or greater than 65%, or greater than 70%, or greater than 75%, or greater than 80%, or greater than 85%, or greater than 90%, or greater than 95%, or greater than 99% of the compound present in crystalline form.

The term “crystallization,” as used throughout this disclosure, can refer to crystallization and/or recrystallization, depending upon the applicable circumstances relating to the preparation of the salts described herein.

As used herein and unless otherwise indicated, the terms “polymorph” and “polymorphic form,” which are used herein synonymously, refer to solid crystalline forms of a compound or complex. Different polymorphs of the same compound can exhibit different physical, chemical, and/or spectroscopic properties. Different physical properties include, but are not limited to, stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to a thermodynamically more stable polymorph) or both (e.g., tablets of one polymorph are more susceptible to breakdown at high humidity).

Different physical properties of polymorphs and/or salts can affect their processing. For example, one polymorph might be more likely to form solvates or might be more difficult to filter or wash free of impurities than another due to, for example, the shape or size distribution of particles of it.

Polymorphs of a molecule can be obtained by a number of methods known in the art. Such methods include, but are not limited to, melt recrystallization, melt cooling, solvent crystallization, desolvation, rapid evaporation, rapid cooling, slow cooling, vapor diffusion and sublimation. Polymorphs can be detected, identified, classified, and characterized using well-known techniques such as, but not limited to, melting point, differential scanning calorimetry (DSC), thermogravimetry (TGA), X-ray powder diffractometry (XRPD), single crystal X-ray diffractometry, vibrational spectroscopy, solution calorimetry, solid state nuclear magnetic resonance (NMR), infrared (IR) spectroscopy, Raman spectroscopy, hot stage optical microscopy, scanning electron microscopy (SEM), electron crystallography and quantitative analysis, particle size analysis (PSA), surface area analysis, solubility, and rate of dissolution.

As used herein to refer to the spectra or data presented in graphical form (e.g., XRPD, DSC, 1R, Raman, and NMR spectra), and unless otherwise indicated, the term “peak” refers to a peak or other special feature that one skilled in the art would recognize as not attributable to background noise.

As used herein and unless otherwise indicated, the term “substantially pure” when used to describe a solid means a solid form of the compound that comprises one or more crystalline polymorphs disclosed herein in at least 50% by weight, and in another embodiment, in at least 60% by weight, and in another embodiment, in at least 70% by weight, and in another embodiment, in at least 75% by weight, and in a further embodiment, in at least 80% by weight, and in another embodiment, in at least 90% by weight, and in a further embodiment, in at least 95% by weight, and in an even further embodiment, at least 97%, or 98%, or 99%, or 100% by weight of the solid.

As used herein and unless otherwise indicated, the term “polymorphically pure” when used to describe a polymorph of a compound, means a solid form of the compound that comprises that polymorph and is substantially free of other polymorphs of the compound. For example, a representative polymorphically pure solid comprises greater than 80% by weight of one polymorphic form of the compound and less than 20% by weight of other polymorphic forms of the compound, while in another embodiment, greater than 90% by weight of one polymorphic form of the compound and less than 10% by weight of other polymorphic forms of the compound, and in a still further embodiment, greater than 95% by weight of one polymorphic form of the compound and less than 5% by weight of other polymorphic forms of the compound, and in an even further embodiment, greater than 97% by weight of one polymorphic form of the compound and less than 3% by weight of other polymorphic forms of the compound.

The term “enantiomerically pure” when referring to a salt or freebase herein refers to the salt or freebase being present predominantly in one enantiomer and substantially free of the other enantiomer. For example, an enantiomerically pure salt or freebase comprises greater than 80% by weight of one enantiomeric form of the salt or freebase, and less than 20% by weight of the other enantiomer of the salt or freebase, while in another embodiment, greater than 90% by weight of one enantiomer and less than 10% by weight of the other enantiomer of the salt or freebase, and in a still further embodiment, greater than 95% by weight of one enantiomer and less than 5% by weight of the other enantiomer of the salt or freebase, and in an even further embodiment, greater than 97% by weight of one enantiomer and less than 3% by weight of the other enantiomer or the salt or freebase.

The term “pharmaceutically acceptable” (such as in the recitation of a pharmaceutically acceptable excipient or carrier) refers to a material that is compatible with administration to a human subject, e.g., the material does not cause an undesirable biological effect. Examples of pharmaceutically acceptable excipients are described in the “Handbook of Pharmaceutical Excipients,” Rowe et al., Ed. (Pharmaceutical Press, 7th ED., 2012).

The terms “treating” and “treatment” refer to ameliorating, suppressing, eradicating, reducing the severity of, decreasing the frequency of, decreasing the incidence of, reducing the risk of, slowing the progression of damage caused by, delaying the onset of the condition, or improving the quality of life of a human patient or subject suffering from a condition.

The terms “effective amount” or “therapeutically effective amount” refer to an amount of a crystalline salt described herein, a pharmaceutical composition comprising the same, a medicament comprising the same, or another material comprising the same, which is effective to achieve a particular pharmacological and/or physiological effect including, but not limited to, reducing the frequency or severity of sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, and recurrent thoughts of death or suicide; or providing a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof. The precise dosage will vary according to a variety of factors, such as subject-dependent variables (e.g., age, immune system health, clinical symptoms, etc.), the disease or disorder being treated, as well as the route of administration and the pharmacokinetics of the agent being administered.

The term “therapeutic index” used in reference to any salt disclosed herein and associated therapeutic effects and side effects refers to the ratio of the dose of said salt required to induce a particular negative side effect to the dose of said salt required to induce the desired therapeutic effect.

“Patient” or “subject” refers to animals, and can include any mammal, such as humans, rats, mice, cats, dogs, goats, sheep, horses, monkeys, apes, rabbits, cattle, etc. The mammalian subject can be in any stage of development including adults, children, infants, and neonates.

Unless indicated to the contrary, the terms “drugs” and “medicament” are synonymous.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements, but may include other elements not expressly listed or inherent to such process, method, article, or apparatus.

When referring to a solid, the term “substantially comprising crystalline” followed by reference to a compound name, such as 2-(4-fluorophenyl)-2-(methylamino)cyclohexan-1-one, including the stereoisomers, salts, and polymorphs, such as (R)-2-(4-fluorophenyl)-2-(methylamino)cyclohexan-1-one or(S)-2-(4-fluorophenyl)-2-(methylamino)cyclohexan-1-one hydrochloride, fumarate, sulfate, p-toluenesulfonate, methanesulfonate, maleate, phosphate, tartrate, citrate, malate, or hippurate, refers to a solid having at least 50 wt % of the indicated compound in the crystalline state. It may contain other impurities. For example, the compound in an amorphous state may additionally be present or another polymorph may be present, or other impurities may be present, but the sum of these impurities is not more than 50 wt %. In an embodiment, the solid may contain at least 55 wt % of the indicated compound in a crystalline state, and in another embodiment, at least 60 wt % of the indicated compound in a crystalline state, and in a further embodiment, at least 65 wt % of the indicated compound in a crystalline state, and in a still further embodiment, at least 70 wt % of the indicated compound in a crystalline state, and in a further embodiment, at least 75 wt % of the indicated compound in a crystalline state, and in a further embodiment, at least 80 wt % of the indicated compound in a crystalline state, and in a still further embodiment, at least 85 wt % of the indicated compound in a crystalline state, and in another embodiment, at least 90 wt % of the indicated compound in a crystalline state, and in a still further embodiment, at least 95 wt % of the indicated compound in a crystalline state, and in an even further embodiment, at least 99 wt % of the indicated compound in a crystalline state. Thus the amount of the indicated compound present in a crystalline state in the solid may be 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, 99 wt %, or 100 wt %.

When referring to a figure or other graphical representation of data relating to any crystal, solid, polymorph, or a mixture thereof described herein, the terms “substantially”, “substantially as depicted”, and “substantially as shown” mean characterized by the graphical data in the identified figure subject to small variations, for example, variations in peak intensities or peak positions in X-ray diffraction patterns due to factors such as variation in instrumental response, aspect ratio, particle size, experimental error, and variations in sample concentration or purity. Nevertheless, one of skill in the art will readily be capable of comparing the graphical data in the figures herein with graphical data for a second polymorph, crystal, or solid form to confirm whether the two sets of graphical data are characterizing the same material or two different materials. It should be understood that all figures are thus representative of the inherent properties of the polymorph, crystal, or solid form to which they relate and that the particular data presented is substantially as depicted in the figure whether or not the modifier substantially is explicitly included in any description of said figure.

The terms “substantially,” “substantially as depicted,” and “substantially as shown” in reference to a DSC thermogram, refer to the peak temperatures being about the values shown and with the same number of peaks as depicted.

Also, the use of “a” or “an” are employed to describe elements and components described herein. This is done merely for convenience and to give a general sense of the scope of the invention. This description should be read to include one or at least one.

Moreover, the singular also includes the plural and vice versa unless it is obvious that it is meant otherwise.

Further, unless expressly stated to the contrary, “or” refers to an inclusive “or” and not to an exclusive “or.” For example, a condition A or B is satisfied by any one of the following: A is true (or present), and B is false (or not present), A is false (or not present), and B is true (or present), and both A and B are true (or present).

Moreover, the term “and/or” is synonymous with the term “or,” as used herein.

When a range or list of values is expressed, an embodiment includes the endpoint of the ranges and/or list and all the points therebetween. For example, a range of 6 to 9, includes the value 6 and 9 and all values therebetween. Similarly, when values are expressed as approximations, by use of the antecedent “about,” it will be understood that the values range from about the two endpoints, where “about” is defined as herein described. All ranges are inclusive and combinable. Further, reference to values stated in ranges includes each and every value within that range.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present disclosure, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

Unless indicated to the contrary, all percentages are by weight.

The term “salts disclosed herein” or “salt disclosed herein” refers to any of the salts disclosed in the exemplification, including crystalline salts. These salts are pharmaceutically acceptable salts.

The present disclosure relates, in part, to a crystalline salt of 2-(4-fluorophenyl)-2-(methylamino)cyclohexan-1-one, including the stereoisomers thereof, such as (R)-2-(4-fluorophenyl)-2-(methylamino)cyclohexan-1-one and(S)-2-(4-fluorophenyl)-2-(methylamino)cyclohexan-1-one. These include the hydrochloride thereof, the sulfate thereof, the p-toluenesulfonate thereof, the methanesulfonate thereof, the maleate thereof, the phosphate thereof, the tartrate thereof, the fumarate thereof, the citrate thereof, the glycolate thereof, the malate thereof, and the hippurate thereof. These salts exist in various polymorphic forms, as described herein, and are included in the present disclosure.

In an embodiment, the present disclosure relates to solids that are substantially a crystalline form of any of the disclosed salts of 2-(4-fluorophenyl)-2-(methylamino)cyclohexan-1-one, including the stereoisomers thereof, such as (R)-2-(4-fluorophenyl)-2-(methylamino)cyclohexan-1-one. The solid may be polymorphically pure, that is, comprising only one crystalline polymorph described herein, or alternatively, it may comprise a mixture of two or more of the aforementioned crystalline polymorphs. In an embodiment, the solid contains one or more of the crystalline polymorphs with at least one crystalline polymorph described herein present in a quantity of at least 50% by weight, and in a further embodiment, in at least 55% by weight, and in a further embodiment, in at least 60% by weight, and in a further embodiment, in at least 65% by weight, and in another embodiment, in at least 70% by weight, and in a further embodiment, in at least 75% by weight, and in a further embodiment, in at least 80% by weight, and in an even further embodiment, in at least 85% by weight, and in an even further embodiment, in at least 90% by weight, and in an even further embodiment, in at least 95% by weight, and in a further embodiment, in at least 98% by weight, and in a further embodiment, in at least 99% by weight. In another embodiment, the solid comprises a mixture of polymorphs.

In an embodiment, the solid is polymorphically pure. That is, it contains one crystalline polymorph substantially free of other crystalline polymorphs. In addition, regardless of whether the solid is polymorphically pure or contains a mixture, it may contain other materials, such as a pharmaceutically acceptable carrier or adjuvant(s) known in the pharmaceutical arts or optionally non-polymorphic impurities.

In an embodiment, the solid may be substantially pure. In another embodiment, it may be polymorphically pure, and in a still further embodiment, it may be both substantially pure and polymorphically pure. In a further embodiment, it may be polymorphically pure and enantiomerically pure. In a further embodiment, it may be substantially pure, enantiomerically pure, and polymorphically pure.

In addition, in an embodiment, the solid is anhydrous, that is, contains less than 5% by weight water. Thus, in an embodiment, the solid is anhydrous and substantially pure, and in another embodiment, is polymorphically pure and is anhydrous, and in a still further embodiment, is substantially pure, polymorphically pure, and is anhydrous. In a still further embodiment, the solid is substantially pure, enantiomerically pure, polymorphically pure, and is anhydrous.

The salts of the present disclosure are prepared by reacting the free base, such as the R isomer of the free base, with an acid to effectuate the formation of the salt. Since the reactions do not affect the chiral center of the free base, the resulting salt product maintains the stereochemistry of the free base. Examples of acids include hydrochloric acid, sulfuric acid, p-tolunesulfonic acid, methanesulfonic acid, phosphoric acid, tartaric acid, fumaric acid, citric acid, glycolic acid, maleic acid, malic acid, hippuric acid, and the like, and are illustrated are illustrated in the exemplification below. The selection of the solvent system for the crystallization of a salt is based on the solubility of the free base and the acid chosen. The solvent may be a single solvent or a mixture of solvents, including mixtures containing water.

The techniques that can be used for salt crystallization are known to the skilled artisan. They include, but are not limited to, melt recrystallization, melt cooling, solvent crystallization, desolvation, rapid evaporation, rapid cooling, slow cooling, vapor diffusion and sublimation. The resulting crystalline products are dried by techniques known in the art, such as air drying, oven drying, vacuum drying, or evaporation of the solvent, such as water. Polymorphs can be detected, identified, classified, and characterized using well-known techniques such as, but not limited to, melting point, differential scanning calorimetry (DSC), thermogravimetry (TGA), X-ray powder diffractometry (XRPD), single crystal X-ray diffractometry, vibrational spectroscopy, solution calorimetry, solid state nuclear magnetic resonance (NMR), infrared (IR) spectroscopy, Raman spectroscopy, hot stage optical microscopy, scanning electron microscopy (SEM), electron crystallography and quantitative analysis, particle size analysis (PSA), surface area analysis, solubility, and rate of dissolution.

The salts prepared are purified by techniques known in the art, such as chromatography, including column chromatography, HPLC, recrystallization, sublimation, filtration, and the like.

The following non-limiting examples are exemplary for the preparation and isolation of the various salts disclosed herein, but the teachings of the present disclosure are not so limiting.

### EXEMPLIFICATION

The following is a list of abbreviations used in this disclosure.

### LIST OF ABBREVIATIONS

| | |
|---|---|
| ¹H NMR/H-1 NMR | Proton Nuclear Magnetic Resonance |
| 2-Me-THF | 2-Methyltetrahydrofuran |
| DCM | Dichloromethane |
| DMF | N,N-Dimethylforamide |
| DMSO | Dimethyl sulfoxide |
| DSC | Differential Scanning Calorimetry |
| DVS | Dynamic Vapor Sorption |
| EtOAc | Ethyl Acetate |
| EtOH | Ethanol |
| HPLC | High Performance Liquid Chromatography |
| h | hour, hours |
| IPA | Isopropanol |
| iPAc | Isopropyl acetate |
| MeCN (ACN) | Acetonitrile |
| MeOH | Methanol |
| ML | Mother liquor, i.e., the solution remaining after |
| | crystalline solids were removed by filtration |
| mg | milligram, milligrams |
| mL | milliliter, milliliters |
| MTBE | Methyl tert-butyl ether |
| MIBK | Methyl iso-butyl ketone |
| nPA | n-propanol |
| PLM | Polarized Light Microscopy |
| RT | Room Temperature (20-25 °C) |
| Simulated XRPD | XRPD calculated from single crystal structure |
| SM | Starting Material |
| TGA | Thermo-Gravimetric Analysis |
| THF | Tetrahydrofuran |
| WC | wet cake |
| XRPD, PXRD | Powder X-Ray Diffraction |

The following is a listing of the instruments and methods discussed herein.

### Instruments and Methods

Powder X-Ray Diffraction (XRPD)

| | |
|---|---|
| Instrument: | Panalytical Empyrean |
| Parameters: | X-Ray tube Cu (Kα radiation); |
| | Power: 45 kV × 40 mA |
| Scanning range: | 2 to 40 2θ (degree) |
| Step size: | 0.01 degree |
| Scanning speed: | 6.33 degree (2θ) per minute |

Proton Nuclear Magnetic Resonance (¹H NMR)

| | |
|---|---|
| Instrument: | Bruker 400 Ultrashield |
| Solvent: | Methanol-d₄ |

Differential Scanning Calorimetry (DSC)

| | |
|---|---|
| Instrument: | TA Instruments DSC 2500 |
| Parameters: | Ramp 5/10/20 °C per minute, up to 300 °C |

Thermo-Gravimetric Analysis (TGA)

| | |
|---|---|
| Instrument: | TA Instruments TGA 5500 |
| Parameters: | 25 to 300 °C, 10 °C/minute, 50 mL/min N₂ sweep |

Polarized Light Microscopy (PLM)

| | |
|---|---|
| Instrument: | Nikon Eclipse Ci Pol |
| Software: | Nikon NIS Elements |

Dynamic Vapor Sorption (DVS)

| | |
|---|---|
| Instrument: | DVS Intrinsic, Surface Measurement Systems |
| Parameters: | 25 °C, 0-90-0% RH for 2 cycles |

In the examples described below, the peaks are rounded off to the nearest tenths or hundredths. Accordingly, in some cases, it may appear that some peaks have the same value. In those situations, the conflicting peaks having the value are rounded to the nearest hundredth, which is placed in parentheses.

In the examples below, the solids are analyzed by XRPD to determine if they are crystalline and if so, by DSC to determine melting point and TGA to determine if hydrated/solvated.

### Example 1

### (a) Preparation of (R)-2-(4-fluorophenyl)-2-(methylamino)cyclohexan-1-one hydrochloride Form 1 (1R HCl Form 1) (Method 1)

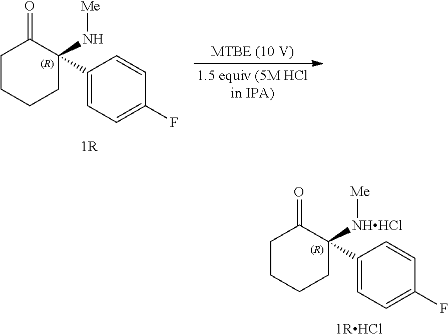

To a solution of (R)-2-(4-fluorophenyl)-2-(methylamino)cyclohexan-1-one free base compound 1R in 10 volumes of MTBE at room temperature was added a solution of 5 N HCl in IPA (1.5 eqiuv.) dropwise, keeping the internal temperature below 30 °C. The formation of a white precipitate was observed upon completion of the addition. The resulting suspension was aged at 20-30 °C for not less than 12 h. The suspension was then filtered, and the cake was washed with 3×3 volumes of MTBE and dried in a vacuum oven at 35-45 °C. The product 1R HCl was obtained as a white solid in 91% yield.

The product of this example will be designated herein as Form 1 or Form 1 polymorph or 1R HCl Form 1 or 1R HCl polymorph Form 1 or Form 1 of (R)-2-(4-fluorophenyl)-2-(methylamino)cyclohexan-1-one hydrochloride or (R)-2-(4-fluorophenyl)-2-(methylamino)cyclohexan-1-one hydrochloride Form 1 or HCl-1. These designations are synonymous and interchangeable.

### (b) Preparation of (R)-2-(4-fluorophenyl)-2-(methylamino)cyclohexan-1-one hydrochloride Form 1 (1R HCl Form 1) (Method 2)

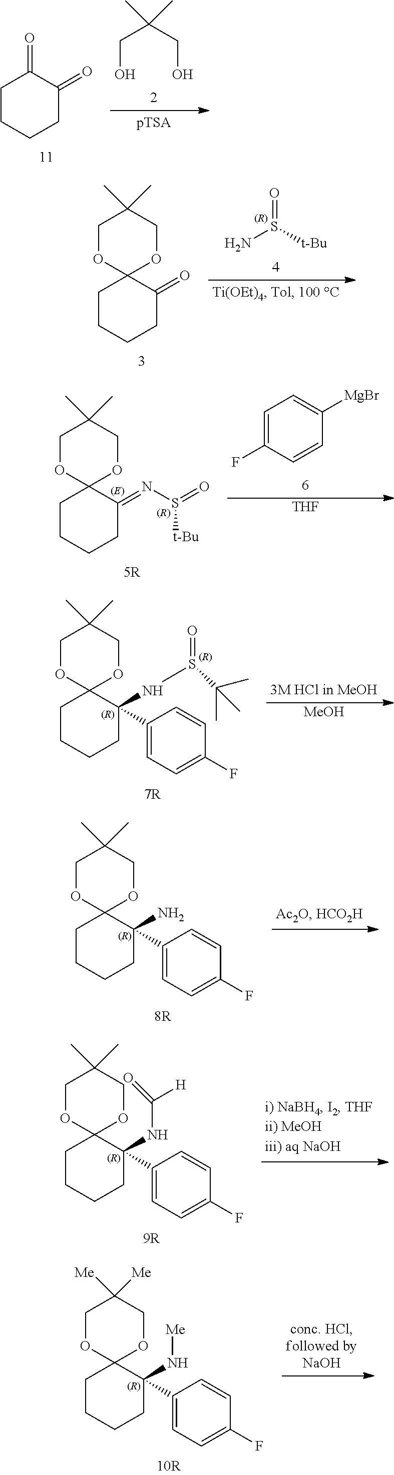

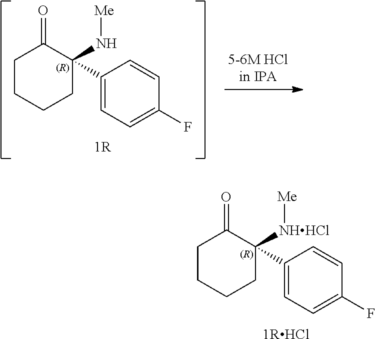

General procedure for the synthesis of 3,3-dimethyl-1,5-dioxaspiro[5.5]undecane-7-one, 3

A 1000 mL jacketed reactor equipped with an over-head stirrer and a Dean-Stark apparatus was charged with 1,2-cyclohexanedione (50.0 g, 428 mmol) (compound 11), 2,2-dimethylpropane-1,3-diol (54.0 g, 514 mmol), p-TSA (1.66 g, 8.6 mmol) and cyclohexane (200 mL) and the resulting suspension was heated at reflux for 3 h to obtain a complete conversion of the starting material. It was then cooled to room temperature and charged with IN NaOH (aq) followed by MTBE and stirred. The phases were separated, and the aqueous phase was further extracted with MTBE and the combined organics were washed once with 10% brine and concentrated. The mixture was azeotroped once with toluene to obtain the 113 g (Q-NMR assay: 66%, yield 87.6%). The crude product was taken to the next step without further purification. ¹H NMR (400 MHZ, CDCl₃) δ 3.55 (d, J=11.1 Hz, 1H), 3.32 (d, J=11.1 Hz, 1H), 2.38-2.35 (m, 2H), 1.8-1.79 (m, 2H), 1.67-1.63 (m, 4H), 1.06 (s, 3H), 0.55 (s, 3H).

General procedure for the synthesis of (R, E)-N-(3,3-dimethyl-1,5-dioxaspiro[5.5]undecan-7-ylidene)-2-methylpropane-2-sulfinamide, 5R

A round bottom flask equipped with an overhead stirrer was charged with compound 3 (33.3 g, 60% wt. %, 0.101 mol), (R)-t-Bu-Sulfinamide (4) (14.62 g, 0.121 mol), toluene (80 mL) and Ti(OEt₄) (25.31 mL, 0.121 mol) at room temperature. The mixture was heated at 80 °C for 5-6 h followed by cooling to room temperature to obtain a dark solution. To this solution was added EDTE (47.5 g), and the mixture was heated at 55 °C for 60 minutes followed by cooling to room temperature. To the above solution was added 12% NaCl (aq) and stirred for about 5 mins and allowed to settle. Phases were separated, and the aqueous phase was re-extracted twice with toluene. The combined organics were washed once with water. The organic phase was filtered through a plug of activated charcoal and SiO₂ and concentrated to obtain the crude product as yellow-orange semi solid (18.9 g net product by NMR wt %, 63%). The product crystalized out as off-white solid upon standing, which was filtered and carried to the next step. ¹H NMR (400 MHZ, CDCl₃) δ 3.83 (d, J=11.0 Hz, 1H), 3.72 (d, J=11.0 Hz, 1H), 3.44-3.38 (m, 2H), 3.13-3.07 (m, 1H), 2.89-2.83 (m, 1H), 1.98-1.85 (m, 1H), 1.81-1.71 (m, 1H), 1.31 (s, 9H), 1.21 (s, 3H), 0.72 (s, 3H).

General procedure for the synthesis of (R)-N-((R)-7-(4-fluorophenyl)-3,3-dimethyl-1,5-dioxaspiro[5.5]undecan-7-yl)-2-methylpropane-2-sulfinamide, 7R

To a stirred solution of compound 5R (17.5 g, 58.0 mmol) in THF (70 mL) at −5 °C was added a 1 M solution of 4-F-Phenyl magnesium bromide in THF (116 mL, 116 mmol, 2 equiv.) dropwise. The resulting reaction mixture was stirred at −5 °C for 4 h followed by room temperature for 14 h. TLC (50% EtOAc/hexanes) indicated the complete conversion of the starting material. The reaction mixture was then cooled to 0 °C and saturated aqueous NH₄Cl (70 mL) was added dropwise. After warming to room temperature, the aqueous phase was extracted with MTBE and the combined organic layer was washed with water followed by drying over Na₂SO₄. Evaporation of the solvent gave the crude product which was re-slurried with heptane followed by filtration to give compound 7R (18.24 g, 79%) as a white solid. ¹H NMR (400 MHZ, CDCl₃) δ 7.72-7.68 (m, 2H), 6.98-6.94 (m, 2H), 4.51 (s, 1H), 3.67-3.60 (m, 2H), 3.35 (dd, J=11.3 Hz and 2.6 Hz, 1H), 3.27 (dd, J=11.3 Hz and 2.5 Hz, 1H), 2.70-2.63 (m, 1H), 2.33-2.27 (m, 1H), 2.05-2.01 (m, 1H), 1.98-1.88 (m, 1H), 1.76-1.66 (m, 1H), 1.62-1.45 (m, 2H), 1.17 (s, 9H), 0.84 (s, 3H), 0.69 (s, 3H)

General procedure for the synthesis of (R)-7-(4-fluorophenyl)-3,3-dimethyl-1,5-dioxaspiro[5.5]undecan-7-amine, 8R

To a suspension of compound 7R (45.0 g, 113 mmol) in methanol (180 mL) at 0 °C was added a solution of 3 M HCl in methanol (113 mL, 339 mmol, 3 equiv.) dropwise. The resulting reaction mixture was allowed to warm to room temperature and stirred for 12-14 h. After completion of the reaction, the mixture was cooled to 0 °C and saturated aqueous NaHCO₃ (225 mL) was added dropwise. To the resulting suspension, CH₂Cl₂ (90 mL) was added to dissolve the product and the phases were separated. The aqueous phase was extracted with CH₂Cl₂ (2×90 mL), and the combined organics were washed with brine and dried (Na₂SO₄) and concentrated to afford the crude compound 8R (27.1 g, 82% quant) as a white solid, which was carried to the next step without further purification. ¹H NMR (400 MHz, CDCl₃) δ 7.62-7.52 (m, 2H), 6.99-6.90 (m, 2H), 3.57 (dd, J=23.4, 11.4 Hz, 2H), 3.16 (ddd, J=11.2, 8.4, 2.7 Hz, 2H), 2.53-2.36 (m, 2H), 1.86-1.34 (m, 8H), 0.59 (s, 3H), 0.36 (s, 3H).

General procedure for the synthesis of (R)—N-(7-(4-fluorophenyl)-3,3-dimethyl-1,5-dioxaspiro[5.5]undecan-7-yl) formamide, 9R

A reactor equipped with stirrer, thermocouple and a nitrogen inlet was charged with Ac₂O (32 mL, 339.56 mmol, 3 equiv.) and HCO₂H (12.8 mL, 339.56 mmol, 3 equiv.) at room temperature. The reaction mixture was heated to 60 °C and aged for 3 h followed by cooling to 0 °C. To the above mixture at 0 °C was added a solution of 8R (26.10 g, 88.96 mmol) in CH₂Cl₂ (125 mL) using an addition funnel, and the mixture was stirred at 0 °C for 1.5-2 h. The HPLC and TLC (30% EtOAc/hexanes) indicated the complete conversion of the starting material to the desired product. The reaction was then quenched with drop-wise addition of a saturated aqueous solution of NaHCO₃ (520 mL) at 0 °C using an addition funnel. The resulting solution was stirred for 30 minutes at 0 °C before being transferred to a separating funnel. The phases were separated, and the aqueous phase was extracted with CH₂Cl₂. The combined organic layer was washed with water and distilled down to a low volume. MeOH was then added and distilled to remove the remaining amount of CH₂Cl₂. The resulting MeOH solution was transferred to a 3-neck round bottom flask and additional MeOH was added and heated at 55 °C for 1 h while stirring. To the above stirring solution was then added water dropwise using an addition funnel over 1 h. The resulting off white suspension was aged at 50 °C for 1 h and allowed to cool to room temperature for 12-14 h. The suspension was then filtered, and the cake was washed with a mixture of 1:1 MeOH/water and dried under suction and then in a vacuum oven for 24 h at 40 °C to obtain compound 9R as a white solid (28.16 g, 98%). ¹H NMR (400 MHZ, CDCl₃) δ 8.29 (d, J=1.9 Hz, 1H), 8.04 (d, J=12.5 Hz, 1H), 7.49-7.37 (m, 2H), 7.03-6.89 (m, 2H), 6.43 (d, J=12.5 Hz, 1H), 6.23 (s, 1H), 3.57 (ddd, J=24.9, 11.4, 7.2 Hz, 2H), 3.24-3.13 (m, 2H), 2.91 (dq, J=13.6, 3.0 Hz, 1H), 2.72-2.51 (m, 2H), 2.42-2.27 (m, 1H), 2.10-1.97 (m, 1H), 1.71-1.59 (m, 3H), 1.58-1.33 (m, 2H), 0.59 (d, 3H), 0.28 (d, J=23.3 Hz, 3H).

General procedure for the synthesis of 10R

A reactor equipped with a chiller, thermocouple and an overhead stirrer was charged with compound 9R (75 g, 0.23 mol) followed by THF and stirred for not less than 15 min. To this reactor was then charged NaBH₄ (26.5 g, 0.7 mmol) portion-wise. The resulting suspension was cooled to −10 °C to 0 °C. A solution of iodine (71.1 g 0.28 mol) in THF was added to the above suspension drop-wise using a dropping funnel, maintaining the internal temperature between −5 °C and 10 °C (Caution: rapid gas evolution). Upon completion of the addition of iodine, the mixture was gradually warmed to 35-45 °C over 30 min and stirred at that temperature for 2-4 h. IPC by HPLC shows the conversion of 9 to 10. The resulting white suspension was cooled to −5 °C to 0 °C and MeOH was added drop-wise over 90 min keeping the internal temperature below 10 °C. Upon completion of the addition, the mixture was heated to 40-45 °C over 30 min and aged at that temperature. The complete decomposition of the intermediate amine borane complex was monitored by 19F NMR studies of the reaction mixture.

In a separate flask, a solution of NaOH (aq) was prepared by dissolving 0.930 g of the NaOH in 9 V of water. A portion (20%, 18 V) of this NaOH (aq) solution was initially added drop-wise to the above reaction mixture at 40-45 °C and aged at this temperature. A formation of a white precipitate was observed immediately upon addition of the NaOH (aq) solution. The rest of the NaOH (aq) solution was then added slowly over 2.5-3 h at 40-45 °C. Upon completion of the addition, the internal temperature of the suspension was set to 20-25 °C and aged at this temperature. The suspension was then filtered, and the cake was washed with a mixture of MeOH/water (1:1) and dried under suction. The cake was then recharged into the reactor followed by water. The resulting suspension was heated to 55-65 °C and aged at this temperature. The suspension was then cooled to 20-25 °C and aged for 30 min before filtering. The cake was then washed with a 1:1 mixture of MeOH/water and dried under suction and then in the vacuum oven at 55-60 °C. The product, compound 10R, was obtained as white solid in 71% (54.8 g) yield with 93.2 wt % by NMR, 3.2% KF and 99.4 A % purity by HPLC. ¹H NMR (400 MHZ, CDCl₃) δ 7.43-7.35 (m, 2H), 7.00-6.92 (m, 2H), 3.56 (dd, J=32.3, 11.1 Hz, 2H), 3.10 (ddd, J=19.8, 11.1, 2.7 Hz, 2H), 2.51-2.39 (m, 1H), 2.28 (td, J=13.3, 3.8 Hz, 1H), 2.04 (s, 3H), 1.87-1.58 (m, 4H), 1.55-1.31 (m, 2H), 0.55 (s, 3H), 0.27 (s, 3H) ppm.

### Preparation of (R)-2-(4-fluorophenyl)-2-(methylamino)cyclohexan-1-one, 1R

A 2 L jacketed reactor equipped with a chiller, thermocouple and an overhead stirrer was charged compound 10R (50 g, 93.4 wt %, 0.152 mol) followed by HPLC water (4 V) at room temperature and the agitation was started. The resulting suspension was cooled to an internal temperature of 15 °C-25 °C. Concentrated HCl (12 N, 4 equiv., 50 mL, 0.76 mol) was added to the above suspension slowly maintaining the temperature below 35 °C. The mixture was heated to an internal temperature of 65-75 °C and aged at this temperature not less than 12 h. Completion of the reaction was monitored by HPLC. The mixture was then cooled to 20-30 °C and MTBE was added thereto and stirred for not less than 10 min. The phases were separated, and the aqueous phase (bottom) was recharged to the reactor. A solution of 3N NaOH (aq) was then added slowly using an addition funnel, keeping the internal temperature below 40 °C, to bring the pH of the mixture 12-13 (measured using a pH paper). The resulting white suspension was extracted with MTBE. The combined MTBE layers were washed with water. The MTBE layer was checked by ¹H NMR for the removal of neopentyl glycol side product (not more than 10%, if >10% repeat the water washings). The MTBE phase was concentrated to 2 V. Additional MTBE was added and concentrated again and this process was repeated two times. The resulted MTBE phase was diluted with MTBE and KF was obtained (KF=not more than 0.2%).

HCl salt formation of 1R

To the above MTBE solution containing the free base compound 1R at room temperature was added a solution of 5-6 N HCl in IPA (45.48 mL, 0.228 mol, 1.5 eqiuv.) drop-wise keeping the internal temperature below 30 °C; a formation of a white precipitate was observed. Upon completion of addition, the resulting suspension was aged at 20-30 °C for not less than 12 h. The suspension was then filtered, and the cake was washed with MTBE (3 V×3, displacement wash, cake wash and slurry wash) and dried under suction for not less than 30 min. The cake was then dried in a vacuum oven at 35-45 °C not less than 12 h. The product 11R·HCl was obtained as white solid in 91% (34.2 g) yield with 99.9 A % HPLC purity, 100.1 wt % by HPLC and 0.152% KF. ¹H NMR (400 MHZ, DMSO-d₆) δ 9.81 (d, J=302.4 Hz, 2H), 7.47-7.28 (m, 4H), 2.44 (p, J=1.9 Hz, 1H), 2.39-2.19 (m, 2H), 2.14 (td, J=13.5, 3.9 Hz, 1H), 2.06 (s, 3H), 1.96-1.85 (m, 1H), 1.85-1.72 (m, 1H), 1.66-1.38 (m, 2H).

Using HPLC, this product was determined to be greater than 99% pure and anhydrous. In addition, it was a crystalline solid.

### Example 2 Preparation of deuterated (R)-2-(4-fluorophenyl)-2-(methylamino)cyclohexan-1-one hydrochloride Form 1 (1R HCl Form 1)

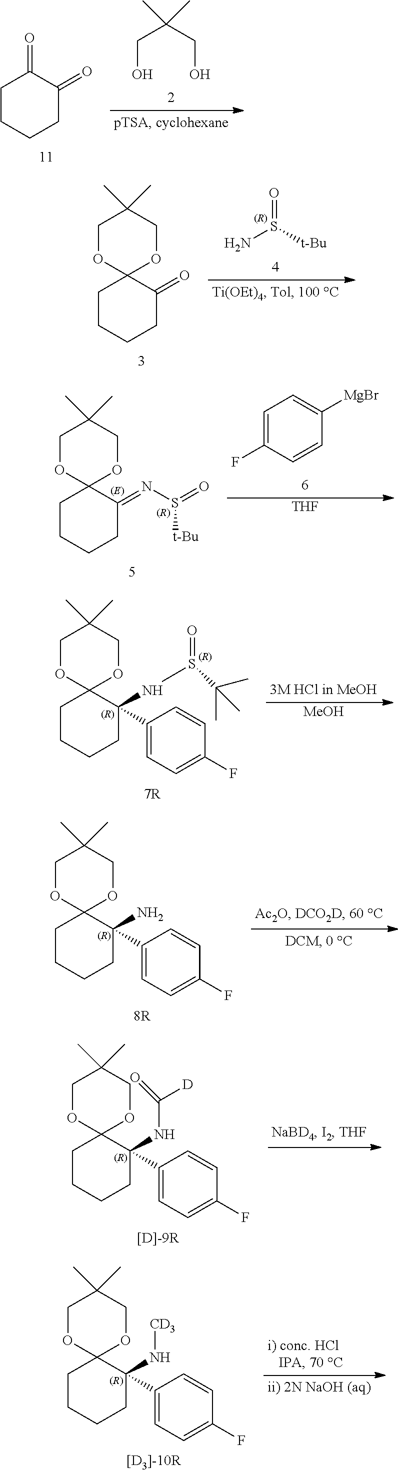

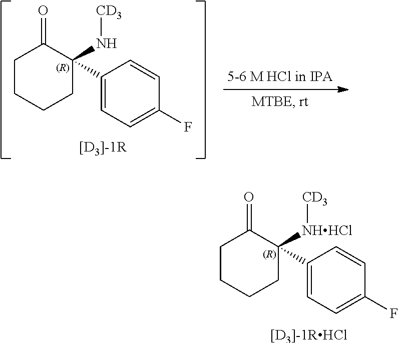

General procedure for the synthesis of 3

A jacketed reactor equipped with stirrer and a Dean-Stark apparatus was charged with 1,2-cyclohexanedione (50.0 g, 428 mmol) (compound 11), 2,2-dimethylpropane-1,3-diol (54.0 g, 514 mmol), p-TSA (1.66 g, 8.6 mmol) and cyclohexane (200 mL, 4 V) and the resulting suspension was heated at reflux for 3 h to obtain a complete conversion of the starting material. It was then cooled to 20 °C and charged with IN NaOH (aq) followed by MTBE and stirred. The phases were separated, and the aqueous phase was further extracted with MTBE and the combined organics were washed once with 10% brine and concentrated. The mixture was azeotroped once with toluene to obtain the 113 g (Q-NMR assay: 66%, yield 87.6%). The crude product was taken to the next step without further purification. ¹H NMR (400 MHZ, CDCl₃) δ 3.55 (d, J=11.1 Hz, 1H), 3.32 (d, J=11.1 Hz, 1H), 2.38-2.35 (m, 2H), 1.8-1.79 (m, 2H), 1.67-1.63 (m, 4H), 1.06 (s, 3H), 0.55 (s, 3H).

General procedure for the synthesis of 5

A 1 L round bottom flask equipped with stirrer was charged with compound 3 (33.3 g, 60% wt. %, 0.101 mol), (R)-t-Bu-Sulfinamide (14.62 g, 0.121 mol) (4), toluene (80 mL) and Ti(OEt₄) (25.31 mL, 0.121 mol) at room temperature. The mixture was heated at 80 °C for 5-6 h followed by cooling to room temperature to obtain a dark solution. To this solution was added EDTE (47.5 g, 2 equiv.) and the mixture was heated at 55 °C for 60 minutes followed by cooling to room temperature. To the above solution was added 12% NaCl (aq) and stirred for about 5 mins and allowed to settle. The phases were separated, and the aqueous phase was re-extracted with toluene. The combined organics were washed with water. The organic phase was filtered through a plug of activated charcoal and SiO₂ and concentrated to obtain the crude product as yellow-orange semi solid (18.9 g net product by NMR; wt % 63%). The product crystalized out as off-white solid upon standing, which was filtered and carried to the next step. ¹H NMR (400 MHZ, CDCl₃) δ 3.83 (d, J=11.0 Hz, 1H), 3.72 (d, J=11.0 Hz, 1H), 3.44-3.38 (m, 2H), 3.13-3.07 (m, 1H), 2.89-2.83 (m, 1H), 1.98-1.85 (m, 1H), 1.81-1.71 (m, 1H), 1.31 (s, 9H), 1.21 (s, 3H), 0.72 (s, 3H).

General procedure for the synthesis of 7R

To a stirred solution of compound 6 (17.5 g, 58.0 mmol) in THF (70 mL) at −5 °C was added a 1 M solution of 4-F-Phenyl magnesium bromide in THF (116 mL, 116 mmol, 2 equiv.) dropwise. The resulting reaction mixture was stirred at −5 °C for 4 h followed by room temperature for 14 h. TLC (50% EtOAc/hexanes) indicated the complete conversion of the starting material. The reaction mixture was then cooled to 0 °C and saturated aqueous NH₄Cl (70 mL) was added dropwise. After warming to the room temperature, the aqueous phase was extracted with MTBE and the combined organic layer was washed with water and then dried over Na₂SO₄. Evaporation of the solvent gave the crude product which was re-slurried with heptane followed by filtration to give compound 7 (18.24 g, 79%) as a white solid. ¹H NMR (400 MHZ, CDCl₃) δ 7.72-7.68 (m, 2H), 6.98-6.94 (m, 2H), 4.51 (s, 1H), 3.67-3.60 (m, 2H), 3.35 (dd, J=11.3 Hz and 2.6 Hz, 1H), 3.27 (dd, J=11.3 Hz and 2.5 Hz, 1H), 2.70-2.63 (m, 1H), 2.33-2.27 (m, 1H), 2.05-2.01 (m, 1H), 1.98-1.88 (m, 1H), 1.76-1.66 (m, 1H), 1.62-1.45 (m, 2H), 1.17 (s, 9H), 0.84 (s, 3H), 0.69 (s, 3H).

General procedure for the synthesis of 8R

To a suspension of compound 7 (45.0 g, 113 mmol) in methanol (180 mL) at 0 °C was added a solution of 3 M HCl in methanol (113 mL, 339 mmol, 3 equiv.) dropwise. The resulting reaction mixture was allowed to warm to room temperature and stirred for 12-14 h. After completion of the reaction, the mixture was cooled to 0 °C and saturated aqueous NaHCO₃ (225 mL) was added dropwise. To the resulting suspension, CH₂Cl₂ was added to dissolve the product and the phases were separated. The aqueous phase was extracted with CH₂Cl₂, and the combined organics were washed with brine and dried (Na₂SO₄) and concentrated to afford the crude compound 8 (27.1 g, 82% quant) as a white solid, which was carried to the next step without further purification. ¹H NMR (400 MHZ, CDCl₃) δ 7.62-7.52 (m, 2H), 6.99-6.90 (m, 2H), 3.57 (dd, J=23.4, 11.4 Hz, 2H), 3.16 (ddd, J=11.2, 8.4, 2.7 Hz, 2H), 2.53-2.36 (m, 2H), 1.86-1.34 (m, 8H), 0.59 (s, 3H), 0.36 (s, 3H)

General procedure for the synthesis of [D]-9R

A mixture of acetic anhydride (1.9 mL, 13.63 mmol) and formic acid-d₃ (0.54 mL, 13.63 mmol) was stirred at 60 °C for 2 h followed by gradually cooling to 0 °C. To the above mixture at 0 °C was then added a solution of compound 8 (1.0 g, 3.41 mmol) in CH₂Cl₂ (5 mL) and the mixture was allowed to stir at 0 °C for 2 h. TLC (30% EtOAc/hexanes) indicated the complete conversion of the starting material. The mixture was then neutralized by slow addition of an aqueous solution of sodium bicarbonate and extracted with CH₂Cl₂. The combined organics were washed once with satd. NaHCO₃(aq), water followed by brine, dried (Na₂SO₄) and concentrated to obtain the crude [D]-9R (1.1 g, quantitative) as off-white solid, which was carried to the next step without further purification. ¹H NMR (400 MHZ, CDCl₃) δ 7.50-7.36 (m, 2H), 7.02-6.88 (m, 2H), 6.56-6.11 (m, 1H), 3.66-3.49 (m, 2H), 3.26-3.11 (m, 2H), 2.98-2.87 (m, 1H), 2.71-2.52 (m, 2H), 2.42-2.29 (m, 1H), 2.11-2.00 (m, 1H), 1.71-1.32 (m, 4H), 0.62-0.57 (m, 3H), 0.33-0.23 (m, 3H).

General procedure for the synthesis of [D₃]-10R

To a stirring suspension of [D]-9R (1.1 g, 3.42 mmol) and NaBD₄ (572 mg, 13.66 mmol) in THF (4 mL) at 0 °C was added a solution of Iodine (1.13 g, 4.44 mmol) in THF (2 mL) drop-wise. The mixture was then allowed to warm to room temperature for 14 h. The mixture was then cooled to 0 °C and quenched with slow addition of MeOH (2 mL) followed by heating at 40 °C for 1 h. The resulting clear solution was then concentrated and treated with MTBE followed by water and IN NaOH (aq) to obtain clear phase separation. The MTBE later was separated, and the aqueous phase was further extracted with MTBE. The combined organics were then washed with water followed by brine, dried (Na₂SO₄) and concentrated. The crude mixture was purified by chromatography on SiO₂ (100% hexane to 30-50% EtO Ac/hexanes) to obtain [D₃]-10 (710 mg, 67%) as white solid. ¹H NMR (400 MHZ, CDCl₃) δ 7.45-7.32 (m, 2H), 7.02-6.90 (m, 2H), 3.56 (dd, J=32.3, 11.1 Hz, 2H), 3.16-3.03 (m, 2H), 2.51-2.41 (m, 1H), 2.27 (td, J=13.3, 3.8 Hz, 1H), 1.86-1.57 (m, 4H), 1.55-1.31 (m, 2H), 0.55 (s, 3H), 0.26 (s, 3H); 19F NMR (376 MHz, CDCl₃) δ -118.7.

General procedure for the synthesis of [D₃]-1R free base

To a solution of [D₃]-10 (640 mg, 2.6 mmol) in IPA (4 V) at room temperature was added a solution of conc. HCL (4 equiv.) and the mixture was heated at 70 °C for 14 h to obtain a complete conversion of the starting material. The mixture was then basified with a solution of 3N NaOH (aq) and extracted with MTBE. The combined organics were washed once with water, dried (Na₂SO₄) and concentrated to obtain crude [D₃]-1R (430 mg, 93%) as colorless oil, which was carried to the next step without further purification.

The terms “deuterated 1R free base” and “[D₃]-1R free base” are synonymous as used herein and will be used interchangeably.

General procedure for the synthesis of [D₃]-1R-HCl salt

To a solution of crude [D₃]-1R free base (430 mg) in MTBE (5 mL) was added a solution of HCl in IPA (1.5 equiv.) drop-wise at room temperature. A formation of a white suspension was observed during the addition of the HCl solution. The resulting white suspension was then allowed to stir at room temperature for 12-14. It was then filtered and washed with MTBE (3×3 V) to obtain the [D₃]-1R-HCl salt (420 mg, 84%) as a white solid. ¹H NMR (400 MHZ, DMSO) δ 9.82 (s, 1H), 9.34 (s, 1H), 7.53-7.32 (m, 4H), 3.15 (dt, J=13.8, 3.0 Hz, 1H), 2.45-2.27 (m, 2H), 2.16-2.03 (m, 1H), 2.02-1.79 (m, 2H), 1.72-1.48 (m, 2H).

As used herein, unless indicated to the contrary, [D₃]-1R-HCl will also be referred to as deuterated 1R hydrochloride.

### Example 3

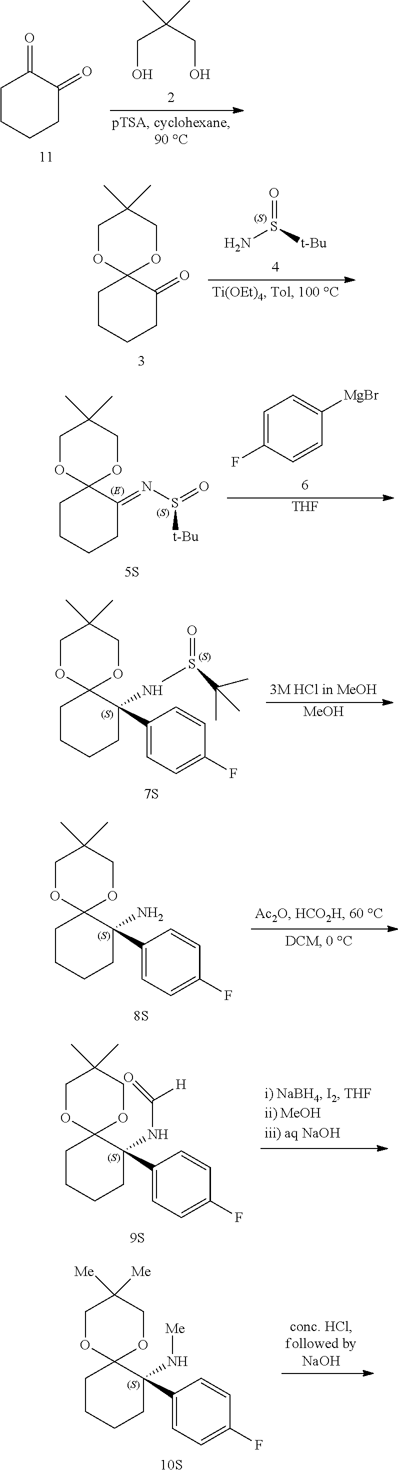

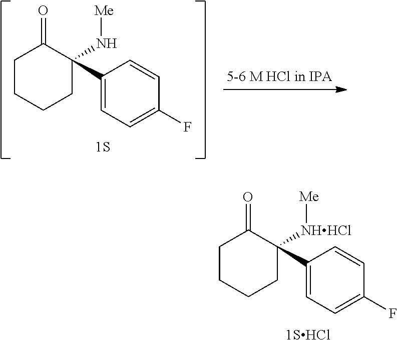

Using the procedures of Example 1 and substituting(S)-t-Bu-sulfinamide for (R)-t-Bu-sulfinamide, the compounds identified in the scheme hereinabove are prepared. As defined herein, the 1S HCl is also denoted herein as 1S hydrochloride.

### Example 4

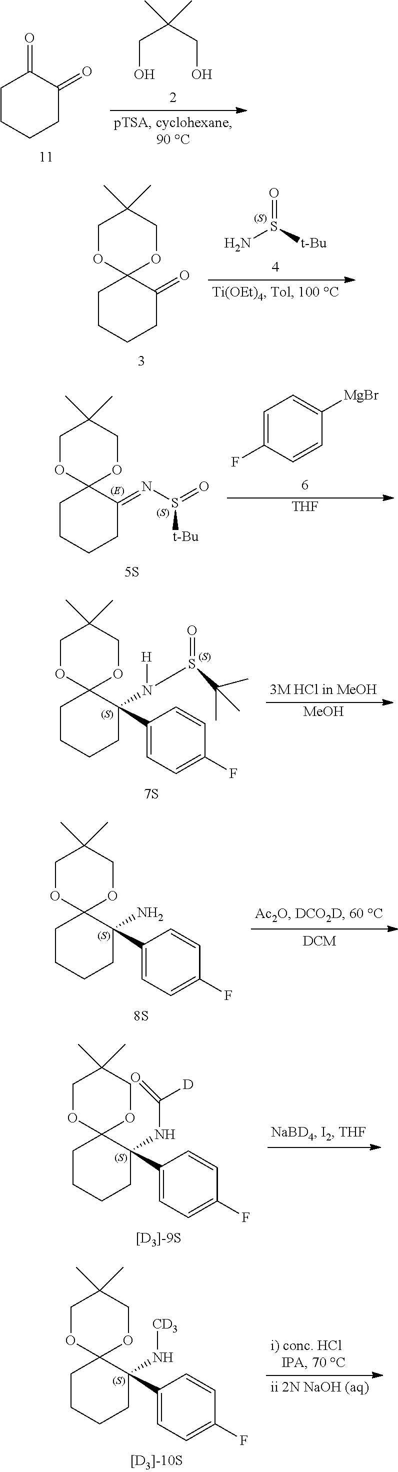

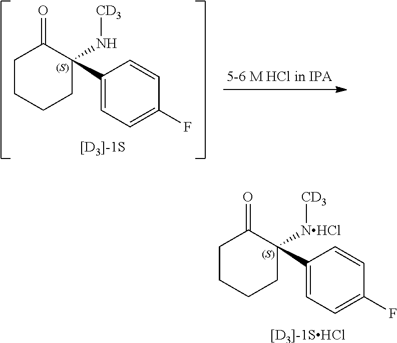

Using the procedures of Example 2 and substituting(S)-t-Bu-sulfinamide for (R)-t-Bu-sulfinamide, the compounds identified in the scheme hereinabove are prepared. As defined herein, [D₃]-1S HCl is also denoted as deuterated 1S hydrochloride.

In the following Examples 5, 6, 7, and 8 (a)-(1), it is to be understood that the data with respect to the R deuterated salts, the S salts and the S deuterated salts are prophetic.

### Example 5. Approximate solubility of the HCl salt of Example 1 (Form 1)

The approximate solubility of the crystalline hydrochloride salt product of Example 1 (1R HCl Form 1) was estimated in 20 solvents as follows: Approximately 10 mg of the product of Example 1 is placed into a 4.0 mL vial, to which 25 μL of solvent at RT is added, and then the vial with its contents is shaken and stirred. If no clear solution is obtained, another 25 μL of solvent is added to the vial and then the vial with its contents is shaken and stirred again. This process is repeated until 4.0 mL solvent is added, and then the contents of the vial are stirred overnight. The results are reported in Table 1 below.

TABLE 1
Approximate solubility of the product
of Example 1 (1R HCl Form 1).

| | | Solvent | b.p. | Solubility | |
|---|---|---|---|---|---|
| Entry | Solvents | Class | (°C) | (mg/mL) | Observation |
| 1 | MeOH | 2 | 64.7 | >486 | clear |
| 2 | EtOH | 3 | 78.37 | 215-431 | clear |
| 3 | IPA | 3 | 82.5 | 50-67 | clear |
| 4 | iPAc | 3 | 89 | <2.4 | slurry |
| 5 | EtOAc | 3 | 77.1 | <2.6 | slurry |
| 6 | nPA | 3 | 97 | 51-69 | clear |
| 7 | ACN | 2 | 82 | 32-38 | clear |
| 8 | MEK | 3 | 79.64 | 3.1-3.7 | clear |
| 9 | MIBK | 2 | 117-118 | <2.7 | slurry |
| 10 | MTBE | 3 | 55.2 | <2.5 | slurry |
| 11 | THF | 2 | 66 | <3.0 | slurry |
| 12 | 1,4-Dioxane | 2 | 101 | 2.9-3.3 | clear |
| 13 | 2-MeTHF | 2 | 80.2 | <2.6 | slurry |
| 14 | Heptane | 3 | 98.42 | <2.7 | slurry |
| 15 | Toluene | 2 | 110.6 | <2.7 | slurry |
| 16 | Water | — | 100 | >464 | clear |
| 17 | Acetone | 3 | 56 | 8.8-9.7 | clear |
| 18 | DCM | 2 | 39.6 | 194-388 | clear |
| 19 | DMF | 2 | 153 | 192-384 | clear |
| 20 | DMSO | 3 | 189 | >395 | clear |

As shown by the results hereinabove, the hydrochloride salt product of Example 1 showed >300 mg/mL solubility in water, MeOH, EtOH, DCM, DMF, and DMSO. Limited or no solubility was seen in esters, ketones, ethers, and alkanes.

### Example 6. Polymorph screening of the 1R HCl salt of Example 1 (Form 1)

Based on the approximate solubility of the product of Example 1 (1R HCl Form 1), screening experiments were conducted to find potential additional crystalline forms (polymorphs) using different screening techniques including, but not limited to, slow evaporation, slow cooling, anti-solvent addition, slurry at RT and 50 °C, temperature cycling, diffusion (solid and liquid), and polymer induced crystallization. The various procedures are outlined hereinbelow.

### 1. Slurry at RT

~40 mg of the product of Example 1 was slurried in 0.5-1.0 mL of different solvents in a 4.0 mL glass vial using a magnetic stirrer at RT. The solids in the slurry were stirred for 7 days.

### 2. Slurry at 50 °C.

~40 mg of the product of Example 1 was slurried in 0.5-1.0 mL of different solvents in a 4.0 mL glass vial using a magnetic stirrer at 50 °C. RT. The solids in the slurry were stirred for 3 days.

3. Anti-solvent addition

~40 mg of the product of Example 1 was dissolved in a solvent to create a saturated solution and an anti-solvent was added up to 10 volume ratios. The solids, if any, were collected.

4. Slow cooling (Thermal cycling)

~40 mg of the product of Example 1 was dissolved in a solvent, heated, and cooled following temperature cycles of 45 °C/1h→35 °C/4h→25 °C/4h→15 °C/4h→5 °C/4 h for 7 days. The obtained solids (if any) were collected after 7 days.

5. Slow evaporation

~40 mg of the product of Example 1 was dissolved in a solvent in a vial to create a saturated solution. The vial opening was covered with parafilm or aluminum foil (2-holes). The vials were kept at RT under the hood. The obtained solids (if any) were collected after 7 days.

6. Liquid vapor diffusion

~40 mg of the product of Example 1 was dissolved in a solvent to create a saturated solution in a 4.0 mL glass vial. The vial was placed in a 20 mL glass vial containing an anti-solvent. The obtained solids were collected after 7-10 days.

7. Solid vapor diffusion

~40 mg of the product of Example 1 was kept in a 4.0 mL glass vial; this vial was placed in a 20 mL glass vial containing a solvent. The solids were collected after 7-10 days.

8. Polymer-induced crystallization.

~40 mg of the product of Example 1 and 2.0 mg of the listed polymer were added to a solvent and kept stirring at RT. The solids in slurry or precipitated solids from solution were collected after 7 days.

The results are provided in Table 2 below.

TABLE 2
Results of polymorph screening of 1R HCl.

| Entry | | | | Crystal |
|---|---|---|---|---|
| No. | Method | Solvent (v:v) | | Form |
| 1 | Slurry at RT | MeOH | | HCl-1 |
| 2 | | IPA | | HCl-1 |
| 3 | | 1,4-dioxane | | HCl-2 |
| 4 | | MIBK | | HCl-1 |
| 5 | | MEK | | HCl-1 |
| 6 | | EtOAc | | HCl-1 |
| 7 | | IPAc | | HCl-1 |
| 8 | | PEG | | amorphous |
| 9 | | Acetone/Toluene (1:1) | | HCl-1 |
| 10 | | THF/n-Heptane (1:1) | | HCl-1 |
| 11 | | MTBE | | HCl-1 |
| 12 | | ACN | | HCl-2 |
| 13 | | Anisole | | HCl-1 |
| 14 | | CPME | | HCl-1 |
| 15 | | Toluene | | HCl-1 |
| 16 | | THF | | HCl-2 |
| 17 | | Acetone/H₂O (a_{w} = 0.2) | | HCl-2 |
| 18 | | Acetone/H₂O (a_{w} = 0.4) | | HCl-2 |
| 19 | | Acetone/H₂O (a_{w} = 0.6) | | gel |
| 20 | | Acetone/H₂O (a_{w} = 0.8) | | gel |
| 21 | | H₂O | | CL |
| 22 | Slurry at 50 °C | EtOH | | HCl-1 |
| 23 | | IPA | | HCl-1 |
| 24 | | 1,4-dioxane | | HCl-1 |
| 25 | | Water | | HCl-1 |
| 26 | | MIBK | | HCl-1 |
| 27 | | MEK | | HCl-1 |
| 28 | | EtOAc | | HCl-1 |
| 29 | | IPAc | | HCl-1 |
| 30 | | 2-MeTHF | | HCl-1 |
| 31 | | Toluene | | HCl-1 |
| 32 | | MTBE | | HCl-1 |
| 33 | | ACN | | HCl-1 |
| 34 | | THF/n-Heptane (1:1) | | HCl-1 |
| 35 | | Acetone/Toluene (1:1) | | HCl-1 |
| 36 | | MeOH | | HCl-1 |
| 37 | | THF/H₂O (9:1) | | gel |
| | | Solvent | Antisolvent | |
| 38 | Anti-solvent | IPA | Acetone | CL |
| 39 | addition | | MTBE | HCl-2 |
| 40 | | | Heptane | HCl-1 |
| 41 | | | 1,4-dioxane | CL |
| 42 | | | IPAc | HCl-2 |
| 43 | | MeOH | THF | HCl-1 |
| 44 | | | MTBE | HCl-1 |
| 45 | | | EtOAc | HCl-1 |
| 46 | | | Acetone | CL |
| 47 | | | Toluene | HCl-2 |
| 48 | | DCM | 1,4-dioxane | CL |
| 49 | | | Acetone | HCl-1 |
| 50 | | | CPME | HCl-1 |
| 51 | | | Hexane | HCl-1 |
| 52 | | | EtOAc | HCl-1 |
| 53 | | Water | THF | CL |
| 54 | | | ACN | CL |
| 55 | | | 1,4-dioxane | CL |
| | | Solvent | | |
| 56 | Thermal cycling | ACN | | HCl-1 |
| 57 | | IPA | | HCl-2 |
| 58 | | DMSO | | HCl-1 |
| 59 | | MeOH/IPAc (1:9) | | HCl-1 |
| 60 | | IPA/Toluene (3:7) | | HCl-2 |
| 61 | | EtOH/n-Heptane (3:7) | | HCl-1 |
| 62 | | EtOH/acetone (3:7) | | gel |
| 63 | Slow | Acetone | | HCl-1 |
| 64 | evaporation | MeOH | | gel |
| 65 | at RT | ACN | | HCl-1 |
| 66 | | DCM | | HCl-2 |
| 67 | | nPA | | HCl-1 |
| 68 | | DMF | | CL |
| 69 | | DMSO | | CL |
| 70 | | NMP | | CL |
| 71 | | Water | | HCl-1 |
| | | Solvent | Antisolvent | |
| 72 | Liquid vapor | MeOH | MTBE | CL |
| 73 | diffusion at RT | | IPAc | CL |
| 74 | | | 2-MeTHF | CL |
| 75 | | DMSO | Acetone | CL |
| 76 | | | Toluene | CL |
| 77 | | | EtOAc | CL |
| 78 | | NMP | Acetone | CL |
| 79 | | | 1,4-dioxane | CL |
| 80 | | | MTBE | HCl-1 |
| | | Solvent | | |
| 81 | Solid vapor | EtOH | | CL |
| 82 | diffusion at RT | Hexane | | HCl-1 |
| 83 | | Acetone | | HCl-1 |
| 84 | | MEK | | HCl-1 |
| 85 | | 2-MeTHF | | HCl-1 |
| 86 | | MeOH | | CL |
| 87 | | MTBE | | HCl-1 |
| 88 | | ACN | | HCl-1 |
| 89 | | Toluene | | HCl-1 |
| 90 | | EtOAc | | HCl-1 |
| 91 | | 1,4-dioxane | | HCl-1 |
| 92 | | DCM | | CL |
| | | Solvent | Polymer | |
| 93 | Polymer induced | MeOH/H₂O (9:1) | PVP | gel |
| 94 | crystallization | Acetone/DCM (9:1) | | HCl-2 |
| 95 | at RT | ACN/H₂O (9:1) | PEO (Poloxamer 407) | gel |
| 96 | | EtOH/Heptane (3/7) | HPMC | HCl-1 |
| 97 | | Acetone/H₂O (9:1) | PVA | HCl-2 |
| 98 | | MTBE/IPA (9:1) | | HCl-1 |

CL = Clear Liquid
HCl-1 = Form 1 polymorph
HCl-2 = Mixture of Polymorphs of Form 1 and Form 2

The data above show that there are at least two hydrochloride polymorph salts, which will be identified hereinbelow as Form 1 and Form 2.

In characterizing the polymorphs described herein, various methods, including XRPD patterns with various peaks are depicted. The peaks observed in the figures and assignments listed herein are intended to encompass variations of ±0.5 ° 2θ. Thus, the values of peaks observed in the figures include the given values ±0.1 °2θ, ±0.2 ° 2θ, ±0.3 ° 2θ, ±0.4 ° 2θ, and ±0.5 ° 2θ. As such, the XRPD patterns are substantially as shown, with the peaks in the Figures herein and/or tables herein, for example, ±0.5 ° 2θ or other reasonable variation as described above. The entire list of peaks or a subset thereof can be sufficient to characterize the crystalline form or characteristics thereof by a pattern substantially similar that is identifiable by one of ordinary skill using the characterization method depicted within experimental variations.

The Form 1 polymorphs from each of the above screening experiments were collected separately, and the XRPD of each was obtained. An XPRD of the HCl Form 1 polymorph, which is exemplary, is depicted in FIG. 2.

The peak values for FIG. 2 are given in Table 3 below

TABLE 3
XRPD peak values for FIG. 2 (HCl Form 1).

| Peak # | Pos. [°2θ] | Intensity |
|---|---|---|
| 1 | 9.7047 | 1795.15 |
| 2 | 12.7540 | 202.56 |
| 3 | 13.1331 | 1646.90 |
| 4 | 14.2840 | 431.37 |
| 5 | 14.6188 | 724.91 |
| 6 | 14.9548 | 3391.00 |
| 7 | 15.7487 | 31.84 |
| 8 | 17.1221 | 480.83 |
| 9 | 18.5553 | 1709.97 |
| 10 | 19.1878 | 948.19 |
| 11 | 19.4506 | 135.76 |
| 12 | 22.3505 | 349.21 |
| 13 | 22.4833 | 572.29 |
| 14 | 22.9252 | 104.22 |
| 15 | 23.0939 | 23.77 |
| 16 | 23.7509 | 22.56 |
| 17 | 24.1561 | 1056.12 |
| 18 | 24.4244 | 246.14 |
| 19 | 25.0507 | 586.99 |
| 20 | 25.2516 | 346.96 |
| 21 | 25.6575 | 97.26 |
| 22 | 26.2784 | 93.69 |
| 23 | 26.6406 | 395.77 |
| 24 | 27.1116 | 38.58 |
| 25 | 27.3801 | 171.91 |
| 26 | 28.7633 | 27.37 |
| 27 | 29.5513 | 125.52 |
| 28 | 30.1444 | 109.33 |
| 29 | 30.5425 | 119.19 |
| 30 | 30.8515 | 148.52 |
| 31 | 31.5948 | 513.45 |
| 32 | 32.1302 | 92.97 |
| 33 | 33.0545 | 181.05 |
| 34 | 33.6721 | 144.37 |
| 35 | 33.9668 | 33.68 |
| 36 | 34.2800 | 133.34 |
| 37 | 34.6232 | 10.02 |
| 38 | 35.0265 | 68.60 |
| 39 | 35.3408 | 50.07 |
| 40 | 35.7924 | 15.83 |
| 41 | 36.2865 | 175.98 |
| 42 | 36.4691 | 54.14 |
| 43 | 36.8750 | 78.01 |
| 44 | 37.5893 | 54.21 |
| 45 | 37.9089 | 25.48 |
| 46 | 38.0351 | 49.17 |
| 47 | 38.7831 | 13.09 |

From the data, the most intense peaks are at 9.7, 15.0, and 18.6±0.5 ° 2θ. In a second embodiment, the most intense peaks are present at 9.7, 13.1, 15.0, and 18.6±0.5 ° 2θ. In another embodiment, the most intense peaks are present at 9.7, 13.1, 15.0, 18.6, and 24.2±0.5 ° 2θ. In another embodiment, the most intense peaks are present at 9.7, 13.1, 14.6, 15.0, 18.6, 19.2, 22.5, 24.2, 25.1, and 31.6±0.5 ° 2θ. In another embodiment, the peaks of Form 1 are 9.7, 12.8, 13.1, 14.3, 14.6, 15.0, 15.7, 17.1, 18.6, 19.2, 19.5, 22.4, 22.5, 23.1, 23.8, 24.2, 24.4, 25.1. 25.3, 25.7, 26.3, 26.6, 27.1, 27.4, 28.8, 29.6, 30.1, 30.5, 30.9, 31.6, 32.1, 33.1, 33.7, 34.0, 34.3, 34.6, 35.0, 35.3, 35.8, 36.3, 36.5, 36.9, 37.6, 37.9, 38.0, and 38.8±0.5 ° 2θ. Form 1 crystals have at least 3 of the peak values depicted above with small variation, such as ±0.5 ° 2θ, while in another embodiment, Form 1 crystals have at least 4 of the peak values depicted above with small variation, such as ±0.5 ° 2θ, while in another embodiment, Form 1 crystals have at least 5 of the peak values depicted above with small variation, such as ±0.5 ° 2θ, while in another embodiment, Form 1 crystals have at least 6 of the peak values depicted above with small variation, such as ±0.5 ° 2θ, while in still another embodiment, Form 1 crystals have at least 7 of the peak values depicted above with small variation, such as ±0.5 ° 2θ, while in still another embodiment, Form 1 crystals have at least 10 of the peak values depicted above with small variation, such as ±0.5 ° 2θ, while in still another embodiment, Form 1 crystals have all of the peak values depicted above with small variation, such as ±0.5 ° 2θ.

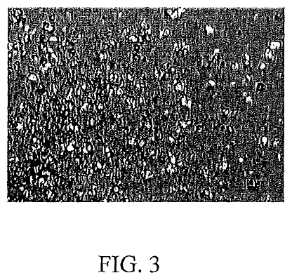

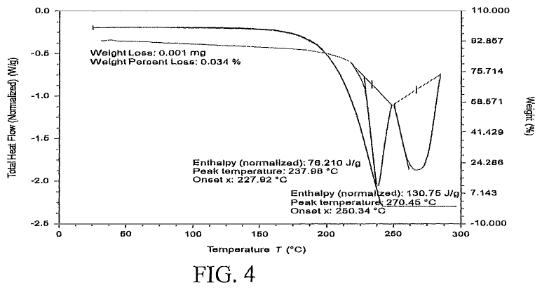

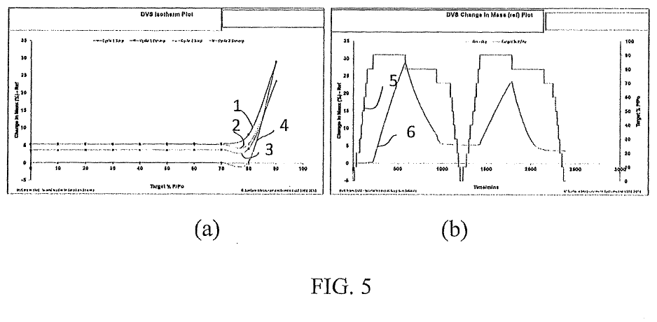

The PLM (polarized light microscopy) of the product of Example 1 is shown in FIG. 3, which shows that the 1R HCl Form 1 solid is an irregular shaped crystalline solid. More specifically, the crystals of Form 1 are orthorhombic. FIG. 4 is a DSC/TGA overlay of 1R HCl Form 1. The first endotherm is about 238.0 °C (peak), and the second peak is about 270.5 °C. Furthermore, as shown in FIG. 4, there is a 0.34% weight loss when 1R HCl Form 1 is heated from room temperature to 100 °C, demonstrating that there is little to no solvent trapped and that the crystal is not a hydrate or solvate.

1R HCl Form 1 was analyzed by DVS (0-90-O-90-0% RH at 25 °C). The DVS behavior is shown in FIGS. 5(a) and (b), wherein 1 plots cycle 1 desorption, 2 plots cycle 2 sorption, 3 plots cycle 2 desorption, 4 plots cycle 1 sorption, 5 plots target % P/Po and 6 plots dm dry line, wherein P is the vapor pressure, Po is the saturation vapor pressure and P/Po is equivalent to % relative humidity for water. The data showed that 1R HCl Form 1 absorbs significant moisture above ~80% RH but readily releases the moisture at lower humidity. It was observed that a post-DVS sample kept at 0% RH for 3 days was the same crystal form as the starting solids.

Moreover, the Form 1 polymorph can be sublimed from a solid phase to gas phase without passing through the liquid phase. The Form 1 polymorph was heated up to 300 °C. The material recovered from sublimation had the same XRPD pattern as 1R HCl Form 1. Polymorph screening of the deuterated 1R HCl salt of Example 2 (Form 1).

Similar XRPD, DSC and DVS patterns can be obtained for the deuterated 1R HCl salt of Example 2. More specifically, subjecting the deuterated R hydrochloride to polymorph screening described hereinabove can produce similar results to that depicted in Table 2. The XRPD of the deuterated R hydrochloride Form 1 are substantially the same as obtained from the XRPD of 1R HCl Form 1. The deuterated R hydrochloride Form 1 is an irregular shaped crystalline solid. The DVS behavior of deuterated 1R hydrochloride Form 1 is substantially the same as that of 1R HCl. Further deuterated 11R hydrochloride Form 1 can be sublimed from the solid phase to the gas phase when heated up to 300 °C. Polymorph screening of the 1S HCl salt of Example 3 (Form 1).

Similar XRPD, DSC and DVS patterns can be obtained for the 1S HCl salt. Subjecting the 1S hydrochloride to polymorph screening described hereinabove produces similar results to that depicted in Table 2. The XRPD of the 1S hydrochloride Form 1 are substantially the same as obtained from the XRPD of 1R HCl Form 1. The 1S hydrochloride Form 1 is an irregular shaped crystalline solid. The DVS behavior of 1S hydrochloride Form 1 is substantially the same as that of 1R HCl. Further deuterated 1R hydrochloride Form 1 can be sublimed from the solid phase to the gas phase when heated up to 300 °C. Polymorph screening of the deuterated 1S HCl salt of Example 4 (Form 1).

Similar XRPD, DSC and DVS patterns can be obtained for the deuterated 1S HCl salt. For example, subjecting the deuterated 1S hydrochloride to polymorph screening described hereinabove produces similar results to that depicted in Table 2. The XRPD of the deuterated 1S hydrochloride Form 1 are substantially the same as obtained from the XRPD of 1R HCl Form 1. The deuterated 1S hydrochloride Form 1 is an irregular shaped crystalline solid. The DVS behavior of deuterated 1S hydrochloride Form 1 is substantially the same as that of 1R HCl. Further deuterated 1S hydrochloride Form 1 can be sublimed from the solid phase to the gas phase when heated up to 300 °C.

### Example 7. Second hydrochloride polymorph

A second polymorph of 1R HCl, namely Form 2, was detected from the screening procedures depicted in Table 2. The second polymorph, 1R HCl, namely Form 2, can be detected in mixtures of solids recovered from suspending ~40 mg of 1R HCl Form 1 in 0.5-1.0 mL of acetonitrile or in acetone/H₂O (a_{w}=0.3) at room temperature. In addition, the Form 2 polymorph can be also detectable in crystal mixtures obtained by dissolving 1R HCl Form 1 in dichloromethane in a vial to create a saturated solution. The vials can be kept under a hood at room temperature for one day, until solids are obtained.

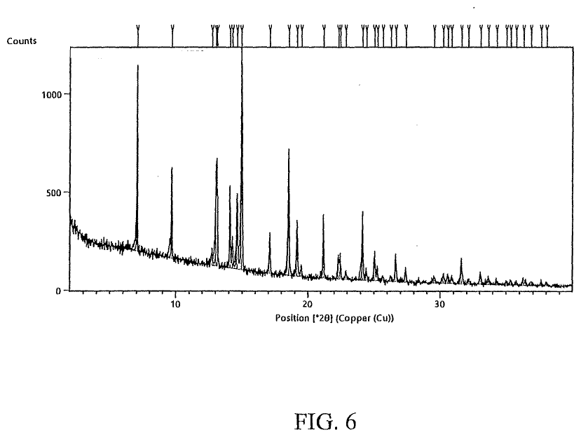

In all three cases, the resulting solid can be characterized by XRPD. From XRPD, it was observed that a second polymorph, 1R HCl Form 2, was created along with 1R HCl Form 1. The solids obtained from these processes, did not enrich in 1R HCl Form 2 over time. However, crystals of Form 2 were physically separable from Form 1 by hand using tweezers to pull particles out under a microscope. The XRPD of a mixture of 1R HCl Form 1 and Form 2 is illustrated in FIG. 6.

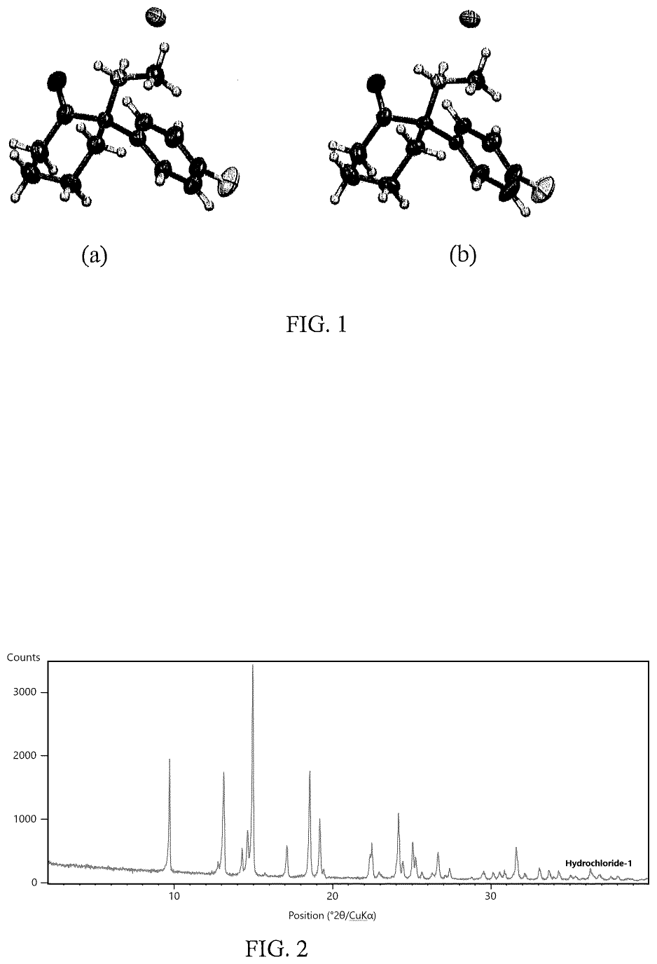

The availability of hand-resolved single crystals allowed for further analysis of Form 2 by single crystal X-ray analysis. FIG. 1 shows the ORTEP drawings of Forms 1 in (a) and 2 in (b) of 1R HCl. The single crystal X-ray diffraction data is shown in Table 4.

TABLE 4
Single crystal X-ray diffraction data
for 1R HCl Form 1 and Form 2.

Form

| Parameter | Form 1 | Form 2 |
|---|---|---|
| Crystal System | Orthorhombic | Monoclinic |
| Space Group | P2₁2₁2₁ | P2₁ |
| a (Å) | 8.1049(4) | 6.7923(7) |
| b (Å) | 12.4123(7) | 7.9112(8) |
| c (Å) | 13.4988(6) | 12.5834(13) |
| α (°) | 90 | 90 |
| β (°) | 90 | 91.688(4) |
| γ (°) | 90 | 90 |
| V (Å³) | 1357.99(12) | 675.88(12) |
| Z | 4 | 2 |
| d (g/cm³) | 1.261 | 1.266 |
| Flack | 0.018(9) | 0.075(17) |
| Parameter | | |

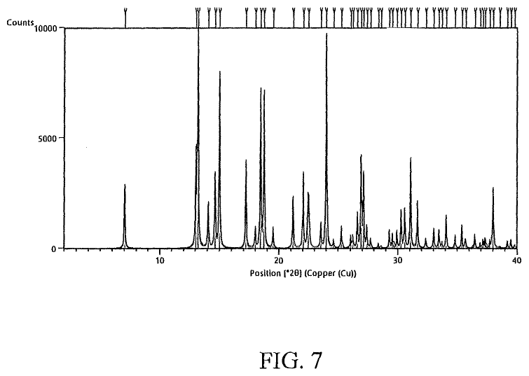

The data clearly distinguishes between the forms. In addition, the simulated XRPD patterns generated from the single X-ray data show very high fidelity to the measured XRPD patterns generated from samples subjected to XRPD. A simulated XPRD pattern of Form 2 is provided in FIG. 7, with the peak values exemplified in FIG. 7 depicted in Table 5 below

TABLE 5
Simulated XRPD peak values for FIG. 7 (HCl Form 2).

| Peak # | Pos. [°2θ] | Intensity |
|---|---|---|
| 1 | 7.0220 | 2879.56 |
| 2 | 13.0329 | 4478.63 |
| 3 | 13.2095 | 10271.09 |
| 4 | 14.0707 | 2102.35 |
| 5 | 14.6311 | 3457.96 |
| 6 | 14.9976 | 8008.08 |
| 7 | 17.1967 | 4020.34 |
| 8 | 18.0047 | 933.85 |
| 9 | 18.4477 | 7291.67 |
| 10 | 18.7413 | 7045.31 |
| 11 | 19.5040 | 983.31 |
| 12 | 21.1734 | 2347.09 |
| 13 | 22.0389 | 3362.57 |
| 14 | 22.4898 | 2489.01 |
| 15 | 23.5588 | 1105.25 |
| 16 | 24.0017 | 9742.52 |
| 17 | 24.6028 | 373.53 |
| 18 | 25.2718 | 985.16 |
| 19 | 26.0510 | 593.05 |
| 20 | 26.2297 | 576.78 |
| 21 | 26.6008 | 1563.23 |
| 22 | 26.9090 | 4244.75 |
| 23 | 27.1100 | 3483.24 |
| 24 | 27.3892 | 1017.16 |
| 25 | 27.7059 | 416.05 |
| 26 | 28.3589 | 220.37 |
| 27 | 28.5878 | 116.92 |
| 28 | 29.2849 | 858.52 |
| 29 | 29.5356 | 665.98 |
| 30 | 29.9144 | 799.43 |
| 31 | 30.2603 | 1677.59 |
| 32 | 30.5677 | 1807.87 |
| 33 | 31.0569 | 4090.47 |
| 34 | 31.6426 | 2123.29 |
| 35 | 32.3508 | 432.41 |
| 36 | 33.0121 | 891.56 |
| 37 | 33.4441 | 821.24 |
| 38 | 33.6924 | 274.89 |
| 39 | 34.0599 | 1460.84 |
| 40 | 34.7962 | 577.93 |
| 41 | 35.3807 | 1024.53 |
| 42 | 35.6908 | 427.35 |
| 43 | 36.4747 | 616.41 |
| 44 | 36.9329 | 238.24 |
| 45 | 37.1642 | 389.76 |
| 46 | 37.3374 | 433.24 |
| 47 | 37.7418 | 337.49 |
| 48 | 38.0096 | 2734.91 |
| 49 | 38.5620 | 59.23 |
| 50 | 39.1832 | 304.65 |
| 51 | 39.4740 | 392.87 |
| 52 | 39.7977 | 149.01 |

From the data, the most intense peaks are at 13.2, 15.0, and 24.0±0.5 ° 2θ. Another set of intense peaks includes 13.2, 15.0, 18.4, 18.7, and 24.0±0.5 ° 2θ. Another set of intense peaks includes 13.0, 13.2, 15.0, 17.2, 18.4, 18.7, 24.0, 26.9, 27.1, and 31.1±0.5 ° 2θ. As shown by the above table, the peaks are located at 7.0, 13.0, 13.2, 14.1, 14.6, 15.0, 17.2, 18.0, 18.4, 18.7, 19.5, 21.2, 22.0, 22.5, 23.6, 24.0, 24.6, 25.3, 26.1, 26.3, 26.6, 26.9, 27.1, 27.4, 27.7, 28.4, 28.6, 29.3, 29.5, 29.9, 30.3, 30.6, 31.1, 31.6, 32.4, 33.0, 33.4, 33.7, 34.1, 34.8, 35.4, 35.7, 36.5, 36.9, 37.2, 37.3, 37.7, 38.0, 38.6, 39.2, 39.5, and 39.8±0.5 ° 2θ.

Second deuterated 1R hydrochloride polymorph.

A second polymorph of the deuterated 1R hydrochloride, namely Form 2, can be detected and is expected to have the same solubilities as in the hydrochloride salts Form 2 depicted in Table 2. For example, it can be detected in mixtures of solids recovered from suspending ~40 mg of deuterated 1R hydrochloride Form 1 in 0.5-1.0 mL of acetonitrile or in acetone/H₂O (a_{w}=0.3) at room temperature. In addition, the Form 2 polymorph is also detectable in crystal mixtures obtained by dissolving deuterated 1R hydrochloride Form 1 in dichloromethane in a vial to create a saturated solution. The vials are kept under a hood at room temperature for one day, until solids were obtained.

The resulting solid can be characterized by XRPD. A second deuterated 1R hydrochloride polymorph, deuterated 1R hydrochloride Form 2, is created along with 1R HCl Form 1. However, crystals of deuterated R hydrochloride Form 2 may be physically separable from Form 1 by hand using tweezers to pull particles out under a microscope. The XRPD of a mixture of deuterated 1R hydrochloride Form 1 and Form 2 is substantially the same as a mixture of Form 1 and Form 2.

The availability of hand-resolved single crystals allows for further analysis of deuterated 1R hydrochloride Form 2 by single crystal X-ray analysis. The ORTEP drawings of the deuterated 1R hydrochloride Forms 1 and 2 are substantially the same as the ORTEP drawings of Forms 1 and 2 of 1R HCl, respectively in FIG. 1. The single crystal X-ray diffraction data with respect to the deuterated 1R hydrochloride Forms 1 and 2 are substantially the same as the single crystal X-ray diffraction data for Forms 1 and 2 of 1R HCl, respectively, the latter of which are depicted in Table 4. Further, the XRPD pattern of a simulated deuterated 1R hydrochloride Form 2 is substantially the same as that of the simulated XRPD pattern of 1R HCl Form 2.

Second S hydrochloride polymorph.

A second polymorph of the 1S hydrochloride, namely Form 2, is expected to have the same solubilities as in the hydrochloride salts Form 2 depicted in Table 2. For example, it can be detected in mixtures of solids recovered from suspending ~40 mg of 1S hydrochloride Form 1 in 0.5-1.0 mL of acetonitrile or in acetone/H₂O (a_{w}=0.3) at room temperature. In addition, the Form 2 polymorph is also detectable in crystal mixtures obtained by dissolving deuterated 1R hydrochloride Form 1 in dichloromethane in a vial to create a saturated solution. The vials are kept under a hood at room temperature for one day, until solids were obtained.

A second polymorph of 1S HCl, namely Form 2, is found. For example, the solubilities of was detectable in mixtures of solids recovered from suspending ~40 mg of 1S HCl Form 2 in 0.5-1.0 mL of acetonitrile or in acetone/H₂O (a_{w}-0.3) at room temperature. In addition, the 1S hydrochloride Form 2 polymorph is also detectable in crystal mixtures obtained by dissolving 1S HCl Form 1 in dichloromethane in a vial to create a saturated solution. The vials are kept under a hood at room temperature for one day, until solids were obtained.

In all cases, the resulting solids can be characterized by XRPD. From XRPD, it can be observed that a second polymorph, 1S HCl Form 2, is created along with 1S HCl Form 1. The solids obtained from these processes, did not enrich in 1S HCl Form 2 over time. However, crystals of Form 2 are physically separable from Form 1 by hand using tweezers to pull particles out under a microscope. The XRPD of a mixture of 1S HCl Form 1 and Form 2 is substantially the same of an XRPD of 1R HCl Form 1 and Form 2. Furthermore, a simulated XPRD pattern of 1S hydrochloride Form 2 is substantially similar to a simulated XRPD pattern of 1R hydrochloride Form 2.

Second deuterated S hydrochloride polymorph

A second polymorph of deuterated 1S HCl, namely Form 2, is detectable in mixtures of solids recovered from suspending ~40 mg of deuterated 1S HCl Form 2 in 0.5-1.0 mL of acetonitrile or in acetone/H₂O (a_{w}=0.3) at room temperature. In addition, the deuterated 1S hydrochloride Form 2 polymorph is also detectable in crystal mixtures obtained by dissolving 1S HCl Form 1 in dichloromethane in a vial to create a saturated solution. The vials are kept under a hood at room temperature for one day, until solids were obtained.

In all cases, the resulting solids can be characterized by XRPD. From XRPD, it can be observed that a second polymorph, deuterated 1S HCl Form 2, is created along with deuterated 1S HCl Form 1. The solids obtained from these processes, did not enrich in deuterated 1S HCl Form 2 over time. However, crystals of Form 2 are physically separable from Form 1 by hand using tweezers to pull particles out under a microscope. The XRPD of a mixture of deuterated 1S HCl Form 1 and Form 2 is substantially the same of an XRPD of 1R HCl Form 1 and Form 2. A simulated XPRD pattern of deuterated 1S Form 2 is substantially similar to a simulated XRPD pattern of 1R hydrochloride Form 2.

### Example 8. Preparation of additional salts

### (a) 1. Preparation of free base (R)-2-(4-fluorophenyl)-2-(methylamino)cyclohexan-1-one and of free base deuterated (R)-2-(4-fluorophenyl)-2-(methylamino)cyclohexan-1-one

The hydrochloride salt prepared in Example 1 is treated with a base, such as NaHCO₃, Na₂CO₃, LiOH, NaOH, KOH, NH₄OH, triethylamine, pyridine, and the like, typically in a solvent, by techniques well known in the art, to form the free base of (R)-2-(4-fluorophenyl)-2-(methylamino)cyclohexan-1-one.

In the following example, the free base was formed by reacting (R)-2-(4-fluorophenyl)-2-(methylamino)cyclohexan-1-one hydrochloride with sodium carbonate.

The hydrochloride salt of (R)-2-(4-fluorophenyl)-2-(methylamino)cyclohexan-1-one prepared as above was mixed with methyl tert-butyl ether (MTBE) at room temperature in a glass beaker or other reaction vessel. A saturated aqueous solution of sodium carbonate was added dropwise with stirring. When the pH of the mixture was approximately 9 or higher, the addition of the saturated sodium carbonate solution was stopped. Additional MTBE was added with distilled water, and the contents of the glass beaker or reaction vessel were shaken and then stirred for an additional hour. The MTBE layer was separated from the water layer and collected. The MTBE was evaporated to provide free base (R)-2-(4-fluorophenyl)-2-(methylamino)cyclohexan-1-one as a solid

Similarly, when the deuterated R hydrochloride prepared in Example 2 is treated with a base, such as NaHCO₃, Na₂CO₃, LiOH, NaOH, KOH, NH₄OH, triethylamine, pyridine, and the like, typically in a solvent, by techniques well known in the art, the deuterated (R)-2-(4-fluorophenyl)-2-(methylamino)cyclohexan-1-one (hereinafter deuterated R free base) is formed.

2. Preparation of free base(S)-2-(4-fluorophenyl)-2-(methylamino)cyclohexan-1-one and of free base deuterated(S)-2-(4-fluorophenyl)-2-(methylamino)cyclohexan-1-one

When the S hydrochloride prepared in Example 3 is treated with a base, such as NaHCO₃, Na₂CO₃, LiOH, NaOH, KOH, NH₄OH, triethylamine, pyridine, and the like, typically in a solvent, by techniques well known in the art, the S 2-(4-fluorophenyl)-2-(methylamino)cyclohexan-1-one (hereinafter S free base) is formed.

Similarly, when the deuterated S hydrochloride prepared in Example 4 is treated with a base, such as NaHCO₃, Na₂CO₃, LiOH, NaOH, KOH, NH₄OH, triethylamine, pyridine, and the like, typically in a solvent, by techniques well known in the art, the deuterated(S)-2-(4-fluorophenyl)-2-(methylamino)cyclohexan-1-one (hereinafter deuterated S free base) is formed.

(b) 1. (R)-2-(4-fluorophenyl)-2-(methylamino)cyclohexan-1-one sulfate

Isopropyl alcohol, ethyl acetate, and tetrahydrofuran (0.25 mL) were added to three different glass vials containing 40 mg of (R)-2-(4-fluorophenyl)-2-(methylamino)cyclohexan-1-one free base, 1 molar equivalent of sulfuric acid was added, and the contents were stirred at room temperature. A solid product was observed from each and was collected and vacuum dried at room temperature. All three products were crystalline, produced the same XRPD pattern, and were designated (R)-2-(4-fluorophenyl)-2-(methylamino)cyclohexan-1-one Sulfate-1 (Sulfate-1). The stoichiometric ratio of base to acid, as determined by proton NMR, was 1:1.

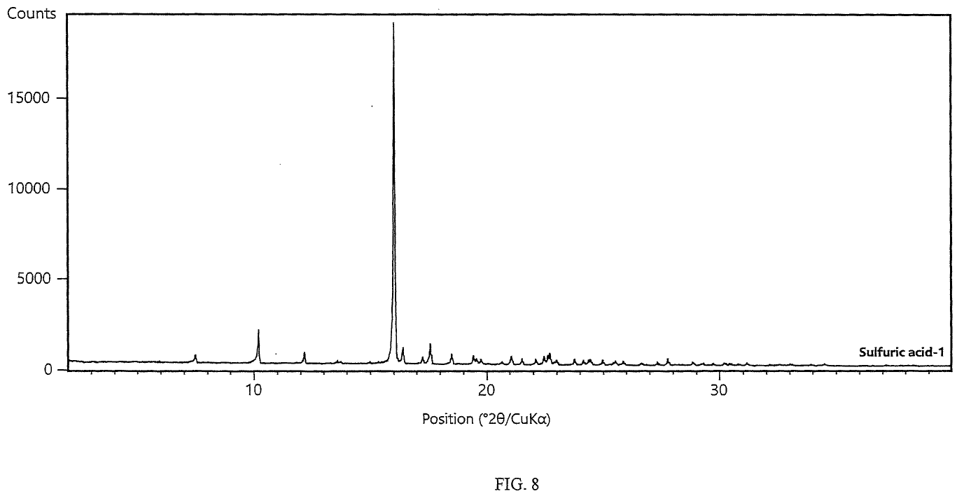

The XRPD diffractogram of Sulfate-1 is depicted in FIG. 8. The peak values and intensity of the XPRD are provided below in Table 6.

TABLE 6
XRPD peak values for FIG. 8 (Sulfate-1).

| Peak # | Pos. [°2θ] | Intensity |
|---|---|---|
| 1 | 7.4790 | 331.22 |
| 2 | 10.2017 | 828.86 |
| 3 | 11.7857 | 29.00 |
| 4 | 12.1650 | 324.51 |
| 5 | 13.5778 | 37.48 |
| 6 | 13.7202 | 114.27 |
| 7 | 14.7837 | 271.60 |
| 8 | 14.9433 | 46.77 |
| 9 | 16.0334 | 4916.97 |
| 10 | 16.3952 | 711.81 |
| 11 | 17.2526 | 181.82 |
| 12 | 17.5712 | 974.62 |
| 13 | 18.4892 | 335.30 |
| 14 | 19.4215 | 436.90 |
| 15 | 19.5275 | 147.30 |
| 16 | 19.7448 | 212.59 |
| 17 | 20.6318 | 84.30 |
| 18 | 21.0391 | 300.55 |
| 19 | 21.5076 | 272.17 |
| 20 | 22.1094 | 119.42 |
| 21 | 22.4488 | 363.65 |
| 22 | 22.6134 | 504.19 |
| 23 | 22.6976 | 755.39 |
| 24 | 22.8724 | 100.94 |
| 25 | 23.0018 | 156.58 |
| 26 | 23.7568 | 282.92 |
| 27 | 24.3633 | 238.27 |
| 28 | 24.9654 | 120.09 |
| 29 | 25.5389 | 155.14 |
| 30 | 25.8598 | 92.83 |
| 31 | 26.6447 | 78.99 |
| 32 | 27.3371 | 68.95 |
| 33 | 27.7738 | 198.01 |
| 34 | 28.7141 | 34.83 |
| 35 | 28.8802 | 119.63 |
| 36 | 29.2868 | 92.44 |
| 37 | 29.7158 | 75.36 |
| 38 | 30.2166 | 112.26 |
| 39 | 30.4631 | 45.01 |
| 40 | 30.8000 | 91.81 |
| 41 | 31.1354 | 116.83 |
| 42 | 32.1500 | 27.23 |
| 43 | 32.3314 | 29.63 |
| 44 | 32.5941 | 56.57 |
| 45 | 32.8005 | 30.28 |
| 46 | 33.0382 | 25.47 |
| 47 | 33.5230 | 23.71 |
| 48 | 33.9309 | 62.28 |
| 49 | 34.3486 | 18.86 |
| 50 | 34.5296 | 60.66 |
| 51 | 38.2099 | 6.08 |
| 52 | 39.0983 | 12.98 |

As shown, in an embodiment, this Sulfate-1 salt can be characterized by intense peaks in the X-Ray diffractogram at 10.2, 16.0, and 17.6±0.5 ° 2θ. In another embodiment, this salt can be characterized by peaks at 10.2, 16.0, 16.4, 17.6, and 22.7±0.5 ° 2θ. In another embodiment, this salt can be characterized by peaks at 7.5, 10.2, 16.0, 16.4, 17.6, 18.5, 19.4, 22.4, 22.6, and 22.7±0.5 ° 2θ. In another embodiment, this salt can be characterized by peaks at 7.5, 10.2, 11.8, 12.2, 13.6, 13.7, 14.8, 14.9, 16.0, 16.4, 17.3, 17.6, 18.5, 19.4, 19.5, 19.7, 20.6, 21.0, 21.5, 22.1, 22.4, 22.6, 22.7, 22.9, 23.0, 23.8, 24.4, 25.0, 25.5, 25.9, 26.6, 27.3, 27.8, 28.7, 28.9, 29.3, 29.7, 30.2, 30.5, 30.8, 31.1, 32.2, 32.6, 32.8, 33.0, 33.5, 33.9, 34.3, 34.5, 38.2, and 39.1±0.5 ° 2θ.

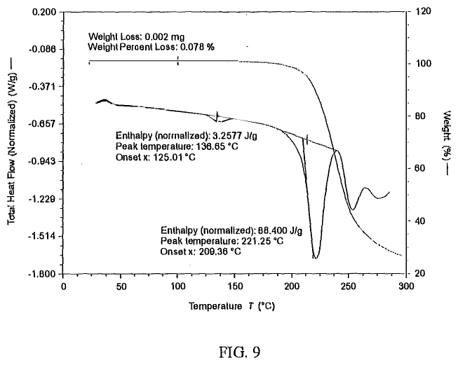

This sulfate salt was analyzed using DSC-TGA with the methodology described hereinabove. The data is depicted in FIG. 9. The weight loss was about 0.08% by 100 °C. In addition, the DSC thermogram of this sulfate salt exhibited two peak temperatures, one small peak temperature at about 136.9 °C, and a more intense one at about 221.3 °C. Other polymorphic forms of the sulfate salts were discovered as follows.

One equivalent of R freebase was placed into a vial to which was added about 1.0 molar equivalent of sulfuric acid. About 10 volumes of ethyl acetate was added thereto to solubilize the reactants and the mixture was stirred at room temperature. After the reaction was completed, the product was collected by filtration.

The XRPD of the wet cake was found to be distinct from that of Sulfate-1 and this new crystalline form was designated (R)-2-(4-fluorophenyl)-2-(methylamino)cyclohexan-1-one Sulfate-2 (Sulfate-2).

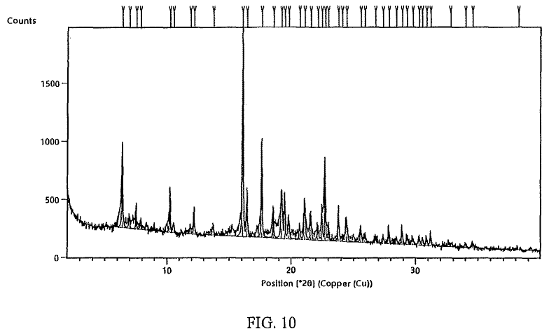

The XRPD diffractogram of the wet cake Sulfate-2 is depicted in FIG. 10. The peak values and intensity of the XPRD are provided below in Table 7.

TABLE 7
XRPD peak values for FIG. 10 (Sulfate-2).

| Peak # | Pos. [°2θ] | Intensity |
|---|---|---|
| 1 | 6.3837 | 680.48 |
| 2 | 6.9482 | 74.53 |
| 3 | 7.5097 | 200.51 |
| 4 | 7.8690 | 54.20 |
| 5 | 10.2270 | 379.03 |
| 6 | 10.5274 | 75.93 |
| 7 | 11.8914 | 48.21 |
| 8 | 12.1819 | 206.50 |
| 9 | 13.7108 | 70.68 |
| 10 | 16.0567 | 1763.62 |
| 11 | 16.4034 | 419.44 |
| 12 | 17.5980 | 842.60 |
| 13 | 18.5327 | 273.47 |
| 14 | 19.1938 | 409.45 |
| 15 | 19.4467 | 400.52 |
| 16 | 19.7672 | 190.09 |
| 17 | 20.6770 | 131.97 |
| 18 | 21.0767 | 358.09 |
| 19 | 21.5696 | 249.26 |
| 20 | 22.1295 | 118.21 |
| 21 | 22.4677 | 309.60 |
| 22 | 22.7159 | 698.27 |
| 23 | 22.9694 | 86.33 |
| 24 | 23.7830 | 306.93 |
| 25 | 24.0741 | 36.65 |
| 26 | 24.4073 | 200.12 |
| 27 | 25.5485 | 127.71 |
| 28 | 25.8846 | 76.07 |
| 29 | 26.7360 | 43.39 |
| 30 | 27.3535 | 51.98 |
| 31 | 27.8144 | 137.75 |
| 32 | 28.4201 | 54.45 |
| 33 | 28.8910 | 137.79 |
| 34 | 29.2673 | 75.09 |
| 35 | 29.7355 | 61.99 |
| 36 | 30.2398 | 76.67 |
| 37 | 30.5070 | 48.54 |
| 38 | 30.8281 | 75.70 |
| 39 | 31.1794 | 106.45 |
| 40 | 32.7534 | 27.59 |
| 41 | 33.9722 | 35.61 |
| 42 | 34.5470 | 41.49 |
| 43 | 38.1801 | 15.03 |

As shown, in an embodiment, this Sulfate-2 salt can be characterized by intense peaks in the X-Ray diffractogram at 16.1, 17.6, and 22.7±0.5 ° 2θ. In another embodiment, this salt can be characterized by peaks at 6.4, 16.1, 16.4, 17.6, and 22.7±0.5 ° 2θ. In another embodiment, this salt can be characterized by peaks at 6.4, 10.2, 16.1, 16.4, 17.6, 19.2, 19.4, 21.1, 22.5, and 22.7. In another embodiment, this salt can be characterized by peaks at 6.4, 6.9, 7.5, 10.2, 10.5, 11.9, 12.2, 13.7, 16.1, 16.4, 17.6, 18.5, 19.2, 19.4, 19.8, 20.7, 21.1, 21.6, 22.1, 22.5, 22.7, 23.0, 23.8, 24.1, 24.4, 25.5, 25.9, 26.7, 27.4, 27.8, 28.4, 28.9, 29.3, 29.7, 30.2, 30.5, 30.8, 31.2, 32.8, 34.0, 34.5, and 38.2±0.5 ° 2θ. The XRPD essentially matched the XRPD pattern of Sulfate-1, but with extra peaks.

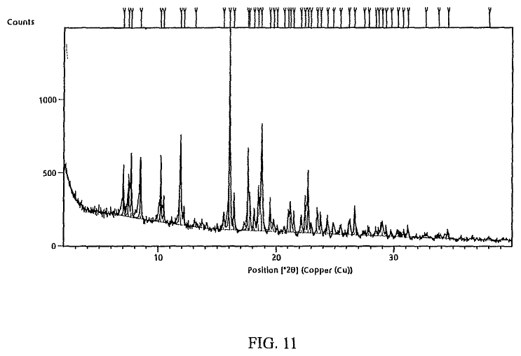

When the wet cake of Sulfate-2 was dried at room temperature, a different XRPD diffractogram was obtained, as shown in FIG. 11, and this new crystalline form was designated as (R)-2-(4-fluorophenyl)-2-(methylamino)cyclohexan-1-one Sulfate-3 (Sulfate-3). The peak values and intensity of the XPRD are provided below in Table 8.

TABLE 8
XRPD peak values for FIG. 11 (Sulfate-3).

| Peak # | Pos. [°2θ] | Intensity |
|---|---|---|
| 1 | 7.0511 | 346.74 |
| 2 | 7.4899 | 272.03 |
| 3 | 7.7099 | 425.27 |
| 4 | 8.4937 | 397.40 |
| 5 | 10.2096 | 457.59 |
| 6 | 10.4535 | 177.10 |
| 7 | 11.8928 | 580.42 |
| 8 | 12.1831 | 116.78 |
| 9 | 13.1427 | 30.14 |
| 10 | 15.5266 | 112.74 |
| 11 | 16.0330 | 1383.81 |
| 12 | 16.4004 | 248.34 |
| 13 | 17.5788 | 538.76 |
| 14 | 17.7077 | 178.78 |
| 15 | 18.1195 | 144.28 |
| 16 | 18.4776 | 303.14 |
| 17 | 18.7374 | 714.27 |
| 18 | 19.4360 | 233.05 |
| 19 | 19.7487 | 77.80 |
| 20 | 20.0596 | 27.09 |
| 21 | 20.6677 | 33.14 |
| 22 | 21.0357 | 143.47 |
| 23 | 21.2178 | 184.22 |
| 24 | 21.5028 | 141.98 |
| 25 | 22.1231 | 118.43 |
| 26 | 22.4544 | 258.57 |
| 27 | 22.7117 | 428.51 |
| 28 | 22.9653 | 48.02 |
| 29 | 23.5141 | 162.68 |
| 30 | 23.7909 | 121.28 |
| 31 | 24.3601 | 101.31 |
| 32 | 24.8665 | 72.48 |
| 33 | 25.4786 | 53.34 |
| 34 | 26.1983 | 85.72 |
| 35 | 26.6571 | 174.51 |
| 36 | 27.4506 | 27.46 |
| 37 | 27.8375 | 48.96 |
| 38 | 28.4353 | 36.04 |
| 39 | 28.6771 | 35.48 |
| 40 | 28.9790 | 83.58 |
| 41 | 29.3087 | 73.39 |
| 42 | 29.7644 | 29.28 |
| 43 | 30.3108 | 42.16 |
| 44 | 30.7525 | 29.58 |
| 45 | 31.1530 | 68.64 |
| 46 | 32.6754 | 22.21 |
| 47 | 33.7337 | 24.53 |
| 48 | 34.5529 | 48.89 |
| 49 | 38.0110 | 15.69 |

As shown, in an embodiment, this Sulfate-3 salt can be characterized by intense peaks in the X-Ray diffractogram at 11.9, 16.0, and 18.7±0.5 ° 2θ. In another embodiment, this salt can be characterized by peaks at 10.2, 11.9, 16.0, 17.6, and 18.7±0.5 ° 2θ. In another embodiment, this salt can be characterized by peaks at 7.1, 7.7, 8.5, 10.2, 11.9, 16.0, 17.6, 18.5, 18.7, and 22.7±0.5 ° 2θ. In another embodiment, this salt can be characterized by peaks at 7.1, 7.5, 7.7, 8.5, 10.2, 10.5, 11.9, 12.2, 13.1, 15.5, 16.0, 16.4, 17.6, 17.7, 18.1, 18.5, 18.7, 19.4, 19.7, 20.1, 20.7, 21.0, 21.2, 21.5, 22.1, 22.5, 22.7, 23.0, 23.5, 23.8, 24.3, 24.9, 25.5, 26.2, 26.7, 27.5, 27.8, 28.4, 28.7, 29.0, 29.3, 29.8, 30.3, 30.8, 31.2, 32.7, 33.7, 34.6, and 38.0±0.5 ° 2θ.

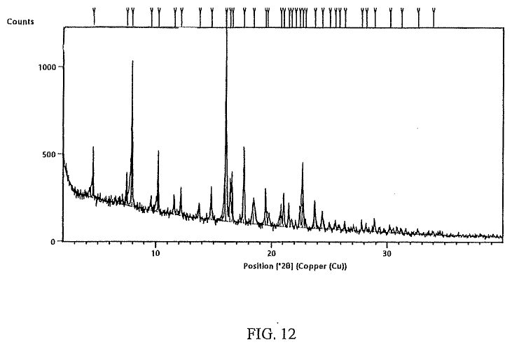

When the Sulfate-3 crystals were further dried with heating set at 40 °C, a different XRPD diffractogram was obtained, as shown in FIG. 12, and this new crystalline form was designated as (R)-2-(4-fluorophenyl)-2-(methylamino)cyclohexan-1-one Sulfate-4 (Sulfate-4). The peak values and intensity of the XPRD are provided below in Table 9.

TABLE 9
XRPD peak values for FIG. 12 (Sulfate-4).

| Peak # | Pos. [°2θ] | Intensity |
|---|---|---|
| 1 | 4.5820 | 270.24 |
| 2 | 7.4799 | 181.11 |
| 3 | 7.9422 | 814.28 |
| 4 | 9.5646 | 55.42 |
| 5 | 10.2038 | 349.52 |
| 6 | 11.5897 | 91.81 |
| 7 | 12.1702 | 157.93 |
| 8 | 13.7585 | 78.57 |
| 9 | 14.7785 | 168.34 |
| 10 | 16.0333 | 1110.38 |
| 11 | 16.4037 | 228.15 |
| 12 | 16.5881 | 234.83 |
| 13 | 17.5685 | 436.17 |
| 14 | 18.4134 | 128.23 |
| 15 | 19.4277 | 196.38 |
| 16 | 19.5870 | 65.29 |
| 17 | 20.8144 | 102.75 |
| 18 | 21.0645 | 186.05 |
| 19 | 21.5143 | 135.19 |
| 20 | 21.7455 | 33.09 |
| 21 | 22.0847 | 23.16 |
| 22 | 22.4535 | 111.64 |
| 23 | 22.7206 | 364.86 |
| 24 | 22.9611 | 34.99 |
| 25 | 23.7622 | 152.80 |
| 26 | 24.3913 | 89.30 |
| 27 | 25.0074 | 30.52 |
| 28 | 25.4685 | 37.62 |
| 29 | 25.8367 | 24.56 |
| 30 | 26.3156 | 45.84 |
| 31 | 27.7878 | 53.47 |
| 32 | 28.1536 | 32.06 |
| 33 | 28.8833 | 70.55 |
| 34 | 30.2147 | 38.25 |
| 35 | 31.2000 | 24.97 |
| 36 | 32.6173 | 24.02 |
| 37 | 33.9454 | 20.23 |

As shown, in an embodiment, this Sulfate-4 salt can be characterized by intense peaks in the X-Ray diffractogram at 7.9, 16.0, and 17.6±0.5 ° 2θ. In another embodiment, this salt can be characterized by peaks at 7.9, 10.2, 16.0, 17.6, and 22.7±0.5 ° 2θ. In another embodiment, this salt can be characterized by peaks at 4.6, 7.9, 10.2, 16.0, 16.4, 16.6, 17.6, 19.4, 21.1, and 22.7±0.5 ° 2θ. In another embodiment, this salt can be characterized by peaks at 4.6, 7.5, 7.9, 9.6, 10.2, 11.6, 12.2, 13.8, 14.8, 16.0, 16.4, 16.6, 17.6, 18.4, 19.4, 19.6, 20.8 21.1, 21.5, 21.7, 22.1, 22.5, 22.7, 23.0, 23.8, 24.4, 25.0, 25.5, 25.8, 26.3, 27.8, 28.2, 28.9, 30.2, 31.2, 32.6, and 33.9±0.5 ° 2θ.

### 2. Preparation of deuterated (R)-2-(4-fluorophenyl)-2-(methylamino)cyclohexan-1-one sulfate

The addition of 1 molar equivalent sulfuric acid to 40 mg of deuterated R free base in an inert solvent, such as isopropyl alcohol, ethyl acetate and tetrahydrofuran (0.25 mL) produces deuterated (R)-2-(4-fluorophenyl)-2-(methylamino)cyclohexan-1-one Sulfate-1 (hereinafter deuterated R sulfate-1), which has substantially the same XRPD diffractogram as that of (R)-2-(4-fluorophenyl)-2-(methylamino)cyclohexan-1-one sulfate-1. The stoichiometric ratio of base to acid is 1:1. The DSC-TGA of deuterated R Sulfate-1 is substantially identical to that of Sulfate-1. In addition, the DVS pattern of deuterated R Sulfate-1 is substantially the same as that of Sulfate-1. Similarly, deuterated (R)-2-(4-fluorophenyl)-2-(methylamino)cyclohexan-1-one sulfate-2 (deuterated R Sulfate-2) is formed by using the same procedure as described hereinabove for forming Sulfate-2 except that (R)-2-(4-fluorophenyl)-2-(methylamino)cyclohexan-1-one free base is substituted with deuterated R freebase. The XRPD of the resulting wet cake of deuterated R Sulfate-2 has substantially the same XRPD diffractogram as Sulfate 2.

Drying the wet cake of deuterated R Sulfate-2 at room temperature produced a new crystalline polymorph, deuterated (R)-2-(4-fluorophenyl)-2-(methylamino)cyclohexan-1-one sulfate-3 (deuterated R Sulfate-3), which exhibits substantially the same XRPD diffractogram as Sulfate-3.

When deuterated R Sulfate-3 crystals were further dried with heating set at 40 °C, a different polymorph identified herein as deuterated (R)-2-(4-fluorophenyl)-2-(methylamino)cyclohexan-1-one sulfate-4 (deuterated R Sulfate-4) is obtained. The XRPD diffractogram of deuterated R Sulfate-4 is substantially the same as that of Sulfate-4.

### 3. Preparation of(S)-2-(4-fluorophenyl)-2-(methylamino)cyclohexan-1-one sulfate and its deuterated analog

The addition of 1 molar equivalent sulfuric acid to 40 mg of S free base in an inert solvent, such as isopropyl alcohol, ethyl acetate and tetrahydrofuran (0.25 mL) produces(S)-2-(4-fluorophenyl)-2-(methylamino)cyclohexan-1-one Sulfate-1 (hereinafter S Sulfate-1), which has substantially the same XRPD diffractogram as that of (R)-2-(4-fluorophenyl)-2-(methylamino)cyclohexan-1-one sulfate-1. The stoichiometric ratio of base to acid is 1:1. The DSC-TGA of S Sulfate-1 is substantially identical to that of Sulfate-1. In addition, the DVS pattern of S Sulfate-1 is substantially the same as that of Sulfate-1.

Similarly, the addition of 1 molar equivalent sulfuric acid to 40 mg of deuterated S free base in an inert solvent, such as isopropyl alcohol, ethyl acetate and tetrahydrofuran (0.25 mL) produces deuterated(S)-2-(4-fluorophenyl)-2-(methylamino)cyclohexan-1-one Sulfate-1 (hereinafter deuterated S Sulfate-1), which has substantially the same XRPD diffractogram as that of (R)-2-(4-fluorophenyl)-2-(methylamino)cyclohexan-1-one sulfate-1. The stoichiometric ratio of base to acid is 1:1. The DSC-TGA of deuterated S Sulfate-1 is substantially identical to that of Sulfate-1. In addition, the DVS pattern of deuterated S Sulfate-1 is substantially the same as that of Sulfate-1.

Other polymorphic forms of the sulfate salt can be discovered as follows.

(S)-2-(4-fluorophenyl)-2-(methylamino)cyclohexan-1-one sulfate-2 (S Sulfate-2) is formed by using the same procedure as described hereinabove for forming Sulfate-2 except that (R)-2-(4-fluorophenyl)-2-(methylamino)cyclohexan-1-one free base is replaced with S freebase. The XRPD of the resulting wet cake of S Sulfate-2 has substantially the same XRPD diffractogram as sulfate 2.

Drying the wet cake of S Sulfate-2 at room temperature produces a new crystalline polymorph, (S)-2-(4-fluorophenyl)-2-(methylamino)cyclohexan-1-one sulfate-3 (S Sulfate-3), which exhibits substantially the same XRPD diffractogram as Sulfate-3. When S Sulfate-3 crystals are further dried with heating set at 40 °C, a different polymorph identified herein as(S)-2-(4-fluorophenyl)-2-(methylamino)cyclohexan-1-one sulfate-4 (S Sulfate-4) is obtained. The XRPD diffractogram of S Sulfate-4 is substantially the same as that of Sulfate-4.

Similarly, deuterated(S)-2-(4-fluorophenyl)-2-(methylamino)cyclohexan-1-one sulfate-2 (deuterated S Sulfate-2) is formed by using the same procedure as described hereinabove for forming Sulfate-2 except that (R)-2-(4-fluorophenyl)-2-(methylamino)cyclohexan-1-one free base is replaced with deuterated S freebase. The XRPD of the resulting wet cake of deuterated S Sulfate-2 has substantially the same XRPD diffractogram as sulfate 2.

Drying the wet cake of deuterated S Sulfate-2 at room temperature produces a new crystalline polymorph, deuterated(S)-2-(4-fluorophenyl)-2-(methylamino)cyclohexan-1-one sulfate-3 (deuterated S Sulfate-3), which exhibits substantially the same XRPD diffractogram as Sulfate-3.

When deuterated S Sulfate-3 crystals are further dried with heating set at 40 °C, a different polymorph identified herein as deuterated(S)-2-(4-fluorophenyl)-2-(methylamino)cyclohexan-1-one sulfate-4 (deuterated S Sulfate-4) is obtained. The XRPD diffractogram of deuterated S Sulfate-4 is substantially the same as that of Sulfate-4.

(c) 1. (R)-2-(4-fluorophenyl)-2-(methylamino)cyclohexan-1-one p-toluenesulfonate and deuterated salts

Ethyl acetate and tetrahydrofuran (0.25 mL) were added to two different glass vials containing 40 mg of (R)-2-(4-fluorophenyl)-2-(methylamino)cyclohexan-1-one free base. One molar equivalent of p-toluenesulfonic acid was added to each vial, and the contents were stirred at room temperature. A solid product was observed in each and each solid was collected separately and vacuum dried at room temperature. The product isolated from ethyl acetate was designated (R)-2-(4-fluorophenyl)-2-(methylamino)cyclohexan-1-one Tosyl-1 (p-toluenesulfonate-1, also denoted as Tosyl-1), while the product isolated from THE was designated as (R)-2-(4-fluorophenyl)-2-(methylamino)cyclohexan-1-one Tosyl-2 (p-toluenesulfonate-2, also denoted as Tosyl-2). In both cases, as determined by proton NMR, the stoichiometric ratio of base to p-toluenesulfonic acid was 1:1.

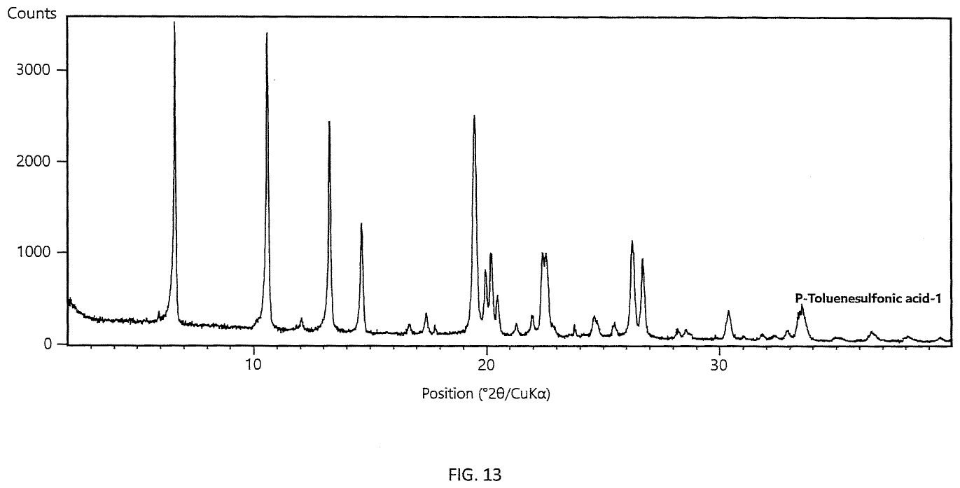

A sample of Tosyl-1 was analyzed by XRPD, and it was found that the diffractogram of Tosyl-1 was different from the diffractogram of Tosyl-2. The Tosyl-1 salt is crystalline. The XRPD diffractogram of Tosyl-1 is depicted in FIG. 13. The peak values and intensity of the XPRD of Tosyl-1 are provided below in Table 10.

TABLE 10
XRPD peak values for FIG. 13 (Tosy1-1).

| | | |
|---|---|---|
| 1 | 2.0946 | 203.60 |
| 2 | 5.9166 | 109.45 |
| 3 | 6.6266 | 3356.40 |
| 4 | 10.5845 | 3310.39 |
| 5 | 12.0444 | 121.09 |
| 6 | 13.2577 | 2388.99 |
| 7 | 14.6313 | 1218.52 |
| 8 | 16.6748 | 106.61 |
| 9 | 17.3993 | 237.55 |
| 10 | 17.7858 | 76.99 |
| 11 | 19.4936 | 2438.57 |
| 12 | 19.9329 | 730.40 |
| 13 | 20.1571 | 890.43 |
| 14 | 20.4393 | 404.23 |
| 15 | 21.2691 | 131.48 |
| 16 | 21.9439 | 222.23 |
| 17 | 22.3835 | 895.69 |
| 18 | 22.5731 | 833.82 |
| 19 | 23.7555 | 132.07 |
| 20 | 24.5947 | 225.10 |
| 21 | 25.4698 | 145.34 |
| 22 | 26.2142 | 994.72 |
| 23 | 26.3131 | 890.16 |
| 24 | 26.6880 | 888.60 |
| 25 | 28.1748 | 84.01 |
| 26 | 28.5466 | 101.75 |
| 27 | 28.9288 | 479.99 |
| 28 | 30.3858 | 323.63 |
| 29 | 31.0175 | 29.93 |
| 30 | 31.8172 | 56.50 |
| 31 | 32.3543 | 48.34 |
| 32 | 32.8867 | 104.97 |
| 33 | 33.3568 | 262.34 |
| 34 | 33.5423 | 385.90 |
| 35 | 35.0579 | 33.83 |
| 36 | 36.5213 | 108.03 |
| 37 | 38.0102 | 41.21 |
| 38 | 39.4847 | 39.57 |

As shown, in an embodiment, this Tosyl-1 salt can be characterized by intense peaks in the X-Ray diffractogram at 6.6, 10.6, and 19.5±0.5 ° 2θ. In another embodiment, this salt can be characterized by peaks at 6.6, 10.6, 13.3, 14.6, and 19.5±0.5 ° 2θ.7. In another embodiment, this salt can be characterized by peaks at 6.6, 10.6, 13.3, 14.6, 19.5, 20.2, 22.4, 26.2, 26.3, and 26.7±0.5 ° 2θ. In another embodiment, this salt can be characterized by peaks at 2.1, 5.9, 6.6, 10.6, 12.0, 13.3, 14.6, 16.7, 17.4, 17.8, 19.5, 19.9, 20.2, 20.4, 21.3, 21.9, 22.4, 22.6, 23.8, 24.6, 25.5, 26.2, 26.3, 26.7, 28.2, 28.5, 28.9, 30.4, 31.0, 31.8, 32.4, 32.9, 33.4, 33.5, 35.1, 36.5, 38.0, and 39.5±0.5 ° 2θ.

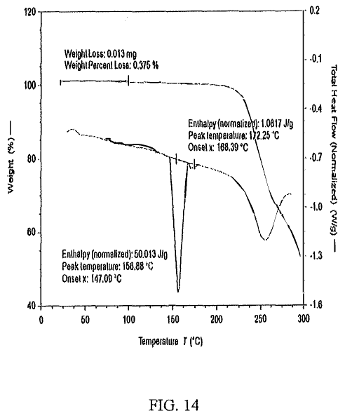

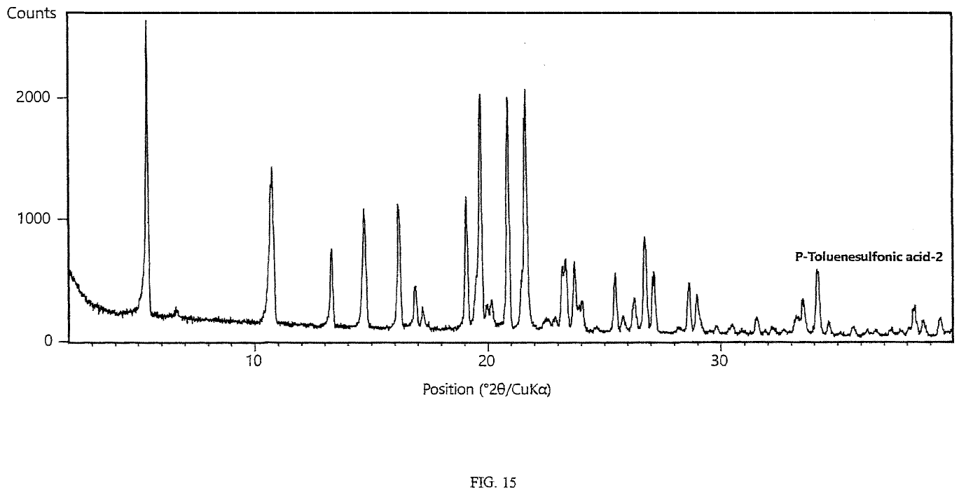

Tosyl-1 was analyzed using DSC-TGA with the methodology described hereinabove. The data is depicted in FIG. 14. The DSC thermogram of this Tosyl-1 salt exhibited two peak temperatures, one intense peak temperature at about 158.9 °C, and a small peak at about 172.3 °C.

The Tosyl-2 salts were also analyzed by XRPD. Tosyl-2 exhibited an XRPD diffractogram in its wet crystal state that was different from its dried cake state. The form corresponding to the diffractogram of the Tosyl-2 wet crystal is designated Tosyl-2A (p-toluenesulfonate-2A) and the form corresponding to the diffractogram of the dry crystal is designated Tosyl-2B (p-toluenesulfonate-2B). The XRPD diffractogram of Tosyl-2A is depicted in FIG. 15. The peak values and intensity of the XPRD of Tosyl-2A are provided below in Table 11.

TABLE 11
XRPD peak values for FIG. 15 (Tosyl-2A).

| Peak # | Pos. [°2θ] | Intensity |
|---|---|---|
| 1 | 5.3752 | 2478.49 |
| 2 | 6.6275 | 65.55 |
| 3 | 10.6754 | 1125.93 |
| 4 | 10.7672 | 1267.72 |
| 5 | 13.2970 | 645.47 |
| 6 | 14.6933 | 987.85 |
| 7 | 16.1413 | 1034.17 |
| 8 | 16.2000 | 895.49 |
| 9 | 16.8613 | 349.17 |
| 10 | 17.1719 | 175.62 |
| 11 | 19.0644 | 1069.94 |
| 12 | 19.7012 | 1889.68 |
| 13 | 19.9607 | 171.54 |
| 14 | 20.1589 | 242.70 |
| 15 | 20.8681 | 1934.33 |
| 16 | 21.5706 | 1762.95 |
| 17 | 21.6381 | 1897.64 |
| 18 | 22.5281 | 90.11 |
| 19 | 22.9033 | 110.03 |
| 20 | 23.1709 | 495.09 |
| 21 | 23.3571 | 546.52 |
| 22 | 23.7044 | 577.73 |
| 23 | 23.8929 | 159.13 |
| 24 | 24.0519 | 248.10 |
| 25 | 24.6488 | 31.40 |
| 26 | 25.4710 | 472.20 |
| 27 | 25.7865 | 123.38 |
| 28 | 26.2837 | 284.29 |
| 29 | 26.6969 | 743.23 |
| 30 | 26.7946 | 596.25 |
| 31 | 27.0530 | 423.96 |
| 32 | 27.1334 | 491.01 |
| 33 | 28.2031 | 36.70 |
| 34 | 28.6418 | 412.73 |
| 35 | 28.9894 | 302.55 |
| 36 | 29.8007 | 57.54 |
| 37 | 30.4933 | 65.29 |
| 38 | 30.9180 | 15.31 |
| 39 | 31.5061 | 125.39 |
| 40 | 32.1803 | 45.59 |
| 41 | 32.6700 | 25.73 |
| 42 | 33.1749 | 106.06 |
| 43 | 33.5120 | 267.27 |
| 44 | 34.0863 | 485.61 |
| 45 | 34.1785 | 489.04 |
| 46 | 34.6128 | 90.89 |
| 47 | 35.6275 | 52.15 |
| 48 | 36.2550 | 33.94 |
| 49 | 36.6607 | 31.40 |
| 50 | 37.2848 | 41.24 |
| 51 | 37.7234 | 27.81 |
| 52 | 38.0742 | 44.66 |
| 53 | 38.2330 | 213.24 |
| 54 | 38.3301 | 239.49 |
| 55 | 38.6720 | 119.77 |
| 56 | 39.3904 | 127.58 |

As shown, in an embodiment, this Tosyl-2A salt can be characterized by intense peaks in the X-Ray diffractogram at 5.4, 20.9, and 21.6 (21.64)±0.5 ° 2θ. In another embodiment, this salt can be characterized by peaks at 5.4, 19.7, 20.9, 21.6 (21.57), and 21.6 (21.63)±0.5 ° 2θ. In another embodiment, this salt can be characterized by peaks at 5.4, 10.7, 10.8, 14.7, 16.1, 19.1, 19.7, 20.9, 21.6 (21.57), and 21.6 (21.63)±0.5 ° 2θ. In another embodiment, this salt can be characterized by peaks at 5.4, 6.6, 10.7, 10.8, 13.3, 14.7, 16.1, 16.2, 16.9, 17.2, 19.1, 19.7, 20.0, 20.2, 20.9, 21.6 (21.57), 21.6 (21.63), 22.5, 23.2, 23.4, 23.7, 23.9, 24.1, 24.6, 25.5, 25.8, 26.3, 26.7, 26.8, 27.1 (27.05), 27.1 (27.13), 28.2, 28.6, 29.0, 29.8, 30.5, 30.9, 31.5, 32.2, 32.7, 33.2, 33.5, 34.1, 34.2, 34.6, 35.6, 36.3, 36.7, 37.3, 37.7, 38.1, 38.2, 38.3, 38.7, and 39.4±0.5 ° 2θ.

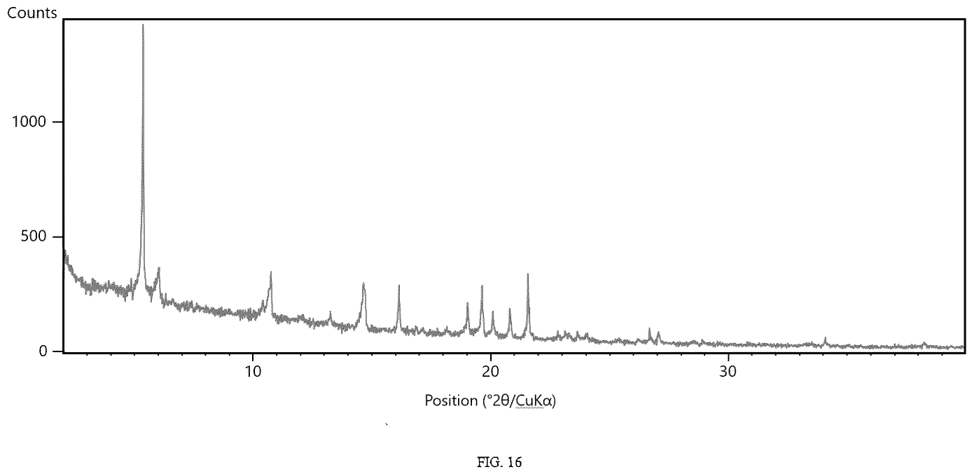

The XRPD diffractogram of Tosyl-2B is depicted in FIG. 16. The peak values and intensity of the XPRD of Tosyl-2B are provided below in Table 12.

TABLE 12
XRPD peak values for FIG. 16 (Tosyl-2B).

| Peak # | Pos. [°2θ] | Intensity |
|---|---|---|
| 1 | 5.3636 | 1209.33 |
| 2 | 6.0194 | 127.42 |
| 3 | 10.4618 | 35.24 |
| 4 | 10.7471 | 203.36 |
| 5 | 13.2468 | 28.61 |
| 6 | 14.6723 | 181.24 |
| 7 | 16.1304 | 187.89 |
| 8 | 19.0221 | 144.65 |
| 9 | 19.2818 | 13.49 |
| 10 | 19.6244 | 223.39 |
| 11 | 20.0794 | 108.46 |
| 12 | 20.8038 | 128.66 |
| 13 | 21.5669 | 280.09 |
| 14 | 23.2046 | 19.94 |
| 15 | 23.9588 | 10.57 |
| 16 | 26.7026 | 34.44 |
| 17 | 27.0654 | 43.80 |
| 18 | 34.1074 | 17.86 |
| 19 | 38.2423 | 9.37 |

As shown, in an embodiment, this Tosyl-2B salt can be characterized by peaks in the X-Ray diffractogram at 5.4, 19.6, and 21.6±0.5 ° 2θ. In another embodiment, this salt can be characterized by peaks at 5.4, 10.7, 16.1, 19.6, and 21.6±0.5 ° 2θ. In another embodiment, this salt can be characterized by peaks at 5.4, 6.0, 10.5, 10.7, 13.2, 14.7, 16.1, 19.0, 19.3, 19.6, 20.1, 20.8, 21.6, 23.2, 23.4, 24.0, 26.7, 26.8, 27.1, 34.1, and 38.2±0.5 ° 2θ.

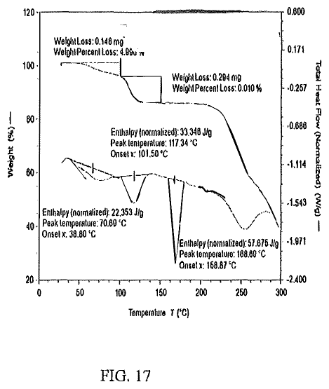

Tosyl-2B was analyzed using DSC-TGA with the methodology described hereinabove. The data is depicted in FIG. 17. The DSC thermogram of this Tosyl-2B salt exhibited three peak temperatures, one intense peak temperature at about 168.7 °C, a small broad peak at about 70.7 °C, and a peak of intermediate intensity at about 117.3 °C.

Substituting deuterated R freebase for (R)-2-(4-fluorophenyl)-2-(methylamino)cyclohexan-1-one free base in the reaction hereinabove with p-toluenesulfonic acid produces two crystalline salts, deuterated (R)-2-(4-fluorophenyl)-2-(methylamino)cyclohexan-1-one p-toluenesulfonate-1 (also designated as deuterated R tosyl-1) and (R)-2-(4-fluorophenyl)-2-(methylamino)cyclohexan-1-one p-toluenesulfonate-2 (also designated as deuterated R tosyl-2), respectively. In both cases, the stoichiometric ratio of base to p-toluenesulfonic acid is 1:1. Moreover, as described hereinabove with respect to Tosyl-2, deuterated R tosyl-2 exhibits an XRPD diffractogram in its wet crystal state that is different from its dried cake state. The form corresponding to the diffractogram of the deuterated R Tosyl-2 wet crystal is designated as deuterated R Tosyl-2A (deuterated R p-toluenesulfonate-2A) and the form corresponding to the diffractogram of the dry crystal is designated as deuterated R Tosyl-2B (deuterated R p-toluenesulfonate-2B) The XRPD and DSC thermogram of deuterated R Tosyl-1 is substantially the same as that of the XRPD and DSC thermogram of Tosyl-1, respectively. The DVS pattern of deuterated R Tosyl-1 is substantially the same as that of Tosyl-1.

The XRPD of deuterated R Tosyl-2A is substantially the same as the XRPD of Tosyl-2A, and the XRPD and DSC-TGA of deuterated R Tosyl-2B is substantially the same as that of the XRPD and DSC-TGA of Tosyl-2B, respectively. The DVS pattern of deuterated R Tosyl-2B is substantially the same as that of Tosyl-2.

### 2. Preparation of(S)-2-(4-fluorophenyl)-2-(methylamino)cyclohexan-1-one p-toluenesulfonate and deuterated salts

Substituting S freebase for (R)-2-(4-fluorophenyl)-2-(methylamino)cyclohexan-1-one free base in the reaction hereinabove with p-toluenesulfonic acid produces two crystalline salts, (S)-2-(4-fluorophenyl)-2-(methylamino)cyclohexan-1-one p-toluenesulfonate-1 (also designated as S tosyl-1) and(S)-2-(4-fluorophenyl)-2-(methylamino)cyclohexan-1-one p-toluenesulfonate-2 (also designated as S tosyl-2), respectively. In both cases, the stoichiometric ratio of base to p-toluenesulfonic acid is 1:1. Moreover, as described hereinabove with respect to Tosyl-2, S Tosyl-2 exhibits an XRPD diffractogram in its wet crystal state that is different from its dried cake state. The form corresponding to the diffractogram of the deuterated S Tosyl-2 wet crystal is designated as S Tosyl-2A (S p-toluenesulfonate-2A) and the form corresponding to the diffractogram of the dry crystal is designated as S Tosyl-2B (S p-toluenesulfonate-2B) The XRPD and DSC thermogram of S Tosyl-1 is substantially the same as that of the XRPD and DSC thermogram of Tosyl-1, respectively. The DVS pattern of S Tosyl-1 is substantially the same as that of Tosyl-1.

The XRPD of S Tosyl-2A is substantially the same as the XRPD of Tosyl-2A, and the XRPD and DSC-TGA of S Tosyl-2B is substantially the same as that of the XRPD and DSC-TGA of Tosyl-2B, respectively. The DVS pattern of S Tosyl-2B is substantially the same as that of Tosyl-2B.

Substituting deuterated S freebase for (R)-2-(4-fluorophenyl)-2-(methylamino)cyclohexan-1-one free base in the reaction hereinabove with p-toluenesulfonic acid produces two crystalline salts, deuterated(S)-2-(4-fluorophenyl)-2-(methylamino)cyclohexan-1-one p-toluenesulfonate-1 (also designated as deuterated S tosyl-1) and(S)-2-(4-fluorophenyl)-2-(methylamino)cyclohexan-1-one p-toluenesulfonate-2 (also designated as deuterated S tosyl-2), respectively. In both cases, the stoichiometric ratio of base to p-toluenesulfonic acid is 1:1. Moreover, as described hereinabove with respect to Tosyl-2, deuterated S tosyl-2 exhibits an XRPD diffractogram in its wet crystal state that is different from its dried cake state. The form corresponding to the diffractogram of the deuterated S Tosyl-2 wet crystal is designated as deuterated S Tosyl-2A (deuterated S p-toluenesulfonate-2A) and the form corresponding to the diffractogram of the dry crystal is designated as deuterated R Tosyl-2B (deuterated R p-toluenesulfonate-2B) The XRPD and DSC thermogram of deuterated S Tosyl-1 is substantially the same as that of the XRPD and DSC thermogram of Tosyl-1, respectively. The DVS pattern of deuterated S Tosyl-1 is substantially the same as that of Tosyl-1.

The XRPD of deuterated S Tosyl-2A is substantially the same as the XRPD of Tosyl-2A, and the XRPD and DSC-TGA of deuterated S Tosyl-2B is substantially the same as that of the XRPD and DSC-TGA of Tosyl-2B, respectively. The DVS pattern of deuterated S Tosyl-2B is substantially the same as that of Tosyl-2B.

(d) 1. (R)-2-(4-fluorophenyl)-2-(methylamino)cyclohexan-1-one methanesulfonate and deuterated R analog

Isopropanol, tetrahydrofuran, and an acetone/water mixture of 9:1 v/v (0.25 mL of each solvent) were added to three different glass vials containing 40 mg of freebase (R)-2-(4-fluorophenyl)-2-(methylamino)cyclohexan-1-one. One molar equivalent of methanesulfonic acid was added to each vial and the contents were stirred at room temperature. A solid product was observed in each and was collected from each and vacuum dried at room temperature. The products isolated from each of the solvents were crystalline and exhibited the same diffractogram when analyzed by XRPD. In all three cases, as determined by proton NMR, the stoichiometric ratio of methanesulfonic acid to base was 1:1.

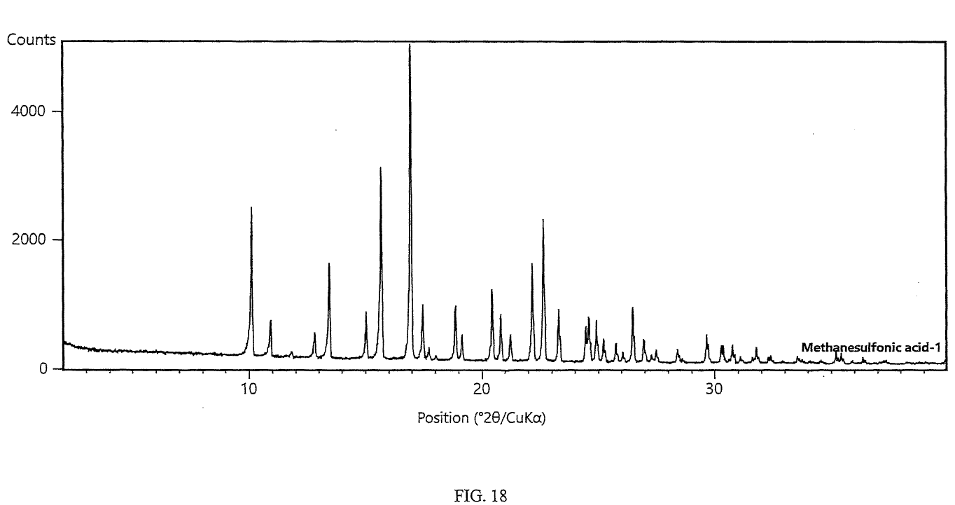

The XRPD diffractograms of the three salts were the same and an exemplary diffractogram form one of the samples is depicted in FIG. 18. The methanesulfonate salt is designated herein as (R)-2-(4-fluorophenyl)-2-(methylamino)cyclohexan-1-one MSA-1 (MSA-1). The peak values and intensity of the XPRD of MSA-1 are provided below in Table 13.

TABLE 13
XRPD peak values for FIG. 18 (MSA-1).

| Peak # | Pos. [°2θ] | Intensity |
|---|---|---|
| 1 | 10.1169 | 2374.71 |
| 2 | 10.9235 | 564.57 |
| 3 | 11.8151 | 80.68 |
| 4 | 12.8166 | 370.89 |
| 5 | 13.4441 | 1507.95 |
| 6 | 15.0248 | 712.53 |
| 7 | 15.6709 | 3037.66 |
| 8 | 16.9353 | 5044.13 |
| 9 | 17.4458 | 864.04 |
| 10 | 17.7179 | 206.00 |
| 11 | 18.0199 | 62.25 |
| 12 | 18.8497 | 869.15 |
| 13 | 19.1233 | 391.01 |
| 14 | 20.4287 | 1126.51 |
| 15 | 20.8011 | 736.28 |
| 16 | 21.2100 | 413.26 |
| 17 | 22.1512 | 1535.32 |
| 18 | 22.3858 | 151.48 |
| 19 | 22.6324 | 2238.04 |
| 20 | 22.7026 | 926.27 |
| 21 | 23.2912 | 791.65 |
| 22 | 24.4534 | 528.23 |
| 23 | 24.5942 | 708.87 |
| 24 | 24.9046 | 647.73 |
| 25 | 25.2222 | 358.89 |
| 26 | 25.7485 | 286.99 |
| 27 | 26.0453 | 159.74 |
| 28 | 26.4687 | 861.60 |
| 29 | 26.9450 | 363.87 |
| 30 | 27.2675 | 122.70 |
| 31 | 27.4732 | 191.07 |
| 32 | 28.4001 | 205.30 |
| 33 | 28.6138 | 30.08 |
| 34 | 29.6406 | 420.10 |
| 35 | 30.2762 | 264.59 |
| 36 | 30.6101 | 34.47 |
| 37 | 30.7612 | 279.11 |
| 38 | 31.0901 | 104.75 |
| 39 | 31.5990 | 90.54 |
| 40 | 31.7777 | 245.98 |
| 41 | 32.2823 | 87.48 |
| 42 | 32.3925 | 109.80 |
| 43 | 33.5295 | 104.20 |
| 44 | 34.5338 | 46.09 |
| 45 | 35.1949 | 190.49 |
| 46 | 35.4195 | 161.86 |
| 47 | 35.8829 | 29.80 |
| 48 | 36.3435 | 84.09 |
| 49 | 37.3129 | 39.81 |
| 50 | 38.2855 | 15.15 |
| 51 | 39.8988 | 63.46 |

As shown, in an embodiment, this MSA-1 salt can be characterized by intense peaks in the X-Ray diffractogram at 10.1, 15.7, and 16.9±0.5 ° 2θ. In another embodiment, this salt can be characterized by peaks at 10.1, 15.7, 16.9, 22.2, and 22.6±0.5 ° 2θ. In another embodiment, this salt can be characterized by peaks at 10.1, 13.4, 15.7, 16.9, 17.4, 18.8, 20.4, 22.2, 22.6, and 22.7±0.5 ° 2θ. In another embodiment, this salt can be characterized by peaks at 10.1, 10.9, 11.8, 12.8, 13.4, 15.0, 15.7, 16.9, 17.4, 17.7, 18.0, 18.8, 19.1, 20.4, 20.8, 21.2, 22.2, 22.4, 22.6, 22.7, 23.3, 24.5, 24.6, 24.9, 25.2, 25.7, 26.0, 26.5, 26.9, 27.3, 27.5, 28.4, 28.6, 29.6, 30.3, 30.6, 30.8, 31.1, 31.6, 31.8, 32.3, 32.4, 33.5, 34.5, 35.2, 35.4, 35.9, 36.3, 37.3, 38.3, and 39.9±0.5 ° 2θ.

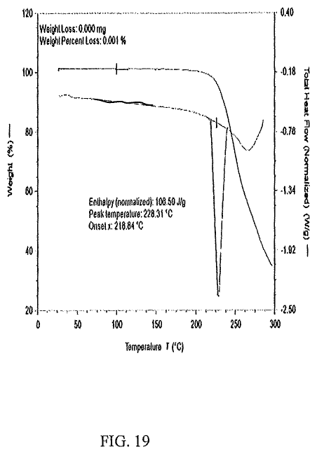

The methanesulfonate salt was analyzed using DSC-TGA with the methodology described hereinabove. The data is depicted in FIG. 19. There was a weight loss of about 0.01% by 100 °C. In addition, the DSC thermogram of the methanesulfonate salt exhibited one intense peak temperature at about 228.3 °C.

Deuterated (R)-2-(4-fluorophenyl)-2-(methylamino)cyclohexan-1-one methanesulfonate (deuterated R MSA-1) is prepared as described hereinabove for MSA-1, except that freebase (R)-2-(4-fluorophenyl)-2-(methylamino)cyclohexan-1-one is replaced with deuterated R free base. The XRPD and DSC-TGA of deuterated R MSA-1 are substantially the same as XRPD and DSC-TGA of MSA-1, respectively. Further, the DVS pattern of deuterated R MSA-1 is substantially the same as that of MSA-1. The stoichiometric ratio of methanesulfonic acid to base is 1:1.

2. (S)-2-(4-fluorophenyl)-2-(methylamino)cyclohexan-1-one methanesulfonate and deuterated S analog

(S)-2-(4-fluorophenyl)-2-(methylamino)cyclohexan-1-one methanesulfonate (S MSA-1) is prepared as described hereinabove for MSA-1, except that R freebase is replaced with S freebase. The XRPD and DSC-TGA of S MSA-1 are substantially the same as XRPD and DSC-TGA of MSA-1, respectively. Further, the DVS pattern of S MSA-1 is substantially the same as that of MSA-1. The stoichiometric ratio of methanesulfonic acid to base is 1:1.

Deuterated(S)-2-(4-fluorophenyl)-2-(methylamino)cyclohexan-1-one methanesulfonate (deuterated S MSA-1) is prepared as described hereinabove for MSA-1, except that freebase(S)-2-(4-fluorophenyl)-2-(methylamino)cyclohexan-1-one is replaced with S deuterated free base. The XRPD and DSC-TGA of deuterated S MSA-1 are substantially the same as XRPD and DSC-TGA of MSA-1, respectively. Further, the DVS pattern of deuterated S MSA-1 is substantially the same as that of MSA-1. The stoichiometric ratio of methanesulfonic acid to base is 1:1.

(e) 1. (R)-2-(4-fluorophenyl)-2-(methylamino)cyclohexan-1-one maleate and deuterated analog

Isopropanol, acetonitrile, ethyl acetate, tetrahydrofuran, and acetone/water mixture of 9:1 v/v (0.25 mL of each solvent) were added to five different glass vials each containing about 40 mg of freebase (R)-2-(4-fluorophenyl)-2-(methylamino)cyclohexan-1-one. One molar equivalent of maleic acid was added to each vial and the contents were stirred at room temperature. A solid product was observed in each and was collected from each and vacuum dried at room temperature. The products isolated from each of the solvents were crystalline. XRPD analysis revealed that two different polymorphs were collected; one polymorph was obtained from acetonitrile and the acetone/water mixture and is identified as (R)-2-(4-fluorophenyl)-2-(methylamino)cyclohexan-1-one Mal-1 (maleate-1, also denoted as Mal-1) and another polymorph was obtained from isopropanol, ethyl acetate, and tetrahydrofuran, and is designated as (R)-2-(4-fluorophenyl)-2-(methylamino)cyclohexan-1-one Mal-2 (maleate-2, also denoted as Mal-2). In all cases, as determined by proton NMR, the stoichiometric ratio of freebase to maleic acid was 1:1.

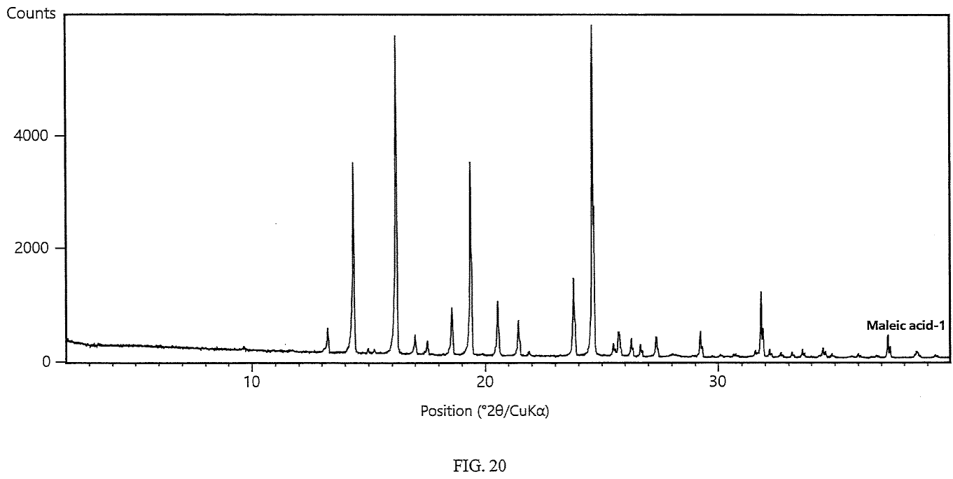

The Mal-1 salt obtained was analyzed by XRPD. The XRPD diffractogram of Mal-1 is depicted in FIG. 20. The peak values and intensity of the XPRD of the Mal-1 salt are provided below in Table 14.

TABLE 14
XRPD peak values for FIG. 20 (Mal-1).

| Peak # | Pos. [°2θ] | Intensity |
|---|---|---|
| 1 | 13.2432 | 420.32 |
| 2 | 14.3468 | 3280.48 |
| 3 | 14.9635 | 79.38 |
| 4 | 15.2352 | 56.35 |
| 5 | 16.1738 | 5527.95 |
| 6 | 16.9774 | 327.00 |
| 7 | 17.5099 | 232.91 |
| 8 | 18.5575 | 816.93 |
| 9 | 19.3647 | 3422.91 |
| 10 | 20.5284 | 937.16 |
| 11 | 21.4095 | 598.99 |
| 12 | 21.8730 | 68.09 |
| 13 | 23.7842 | 1369.25 |
| 14 | 24.5984 | 5687.74 |
| 15 | 24.6734 | 2251.41 |
| 16 | 25.4947 | 229.49 |
| 17 | 25.7065 | 431.43 |
| 18 | 26.2574 | 302.14 |
| 19 | 26.6534 | 209.26 |
| 20 | 27.3344 | 351.79 |
| 21 | 28.0112 | 35.58 |
| 22 | 29.2244 | 444.57 |
| 23 | 29.3093 | 164.44 |
| 24 | 30.6864 | 33.98 |
| 25 | 31.5733 | 109.81 |
| 26 | 31.8199 | 1155.78 |
| 27 | 31.9098 | 474.32 |
| 28 | 32.1809 | 131.15 |
| 29 | 32.6741 | 75.43 |
| 30 | 33.1449 | 87.37 |
| 31 | 33.5895 | 128.44 |
| 32 | 34.4816 | 155.99 |
| 33 | 34.5857 | 98.67 |
| 34 | 34.8459 | 49.64 |
| 35 | 35.7106 | 16.47 |
| 36 | 36.0025 | 67.75 |
| 37 | 36.8268 | 27.08 |
| 38 | 37.2722 | 402.52 |
| 39 | 37.3713 | 188.89 |
| 40 | 38.5350 | 106.58 |
| 41 | 39.3409 | 30.08 |

As shown, in an embodiment, this Mal-1 salt can be characterized by intense peaks in the X-Ray diffractogram at 14.3, 16.2, 19.4, and 24.6±0.5 ° 2θ. In another embodiment, this salt can be characterized by peaks at 14.3, 16.2, 19.4, 24.6, and 24.7±0.5 ° 2θ. In another embodiment, this salt can be characterized by peaks at 14.3, 16.2, 18.6, 19.4, 20.5, 21.4, 23.8, 24.6, 24.7, and 31.8±0.5 ° 2θ. In another embodiment, this salt can be characterized by peaks at 13.2, 14.3, 15.0, 15.2, 16.2, 17.0, 17.5, 18.6, 19.4, 20.5, 21.4, 21.9, 23.8, 24.6, 24.7, 25.5, 25.7, 26.3, 26.7, 27.3, 28.0, 29.2, 29.3, 30.7, 31.6, 31.8, 31.9, 32.2, 32.7, 33.1, 33.6, 34.5, 34.6, 34.8, 35.7, 36.0, 36.8, 37.3, 37.4, 38.5, and 39.3±0.5 ° 2θ.

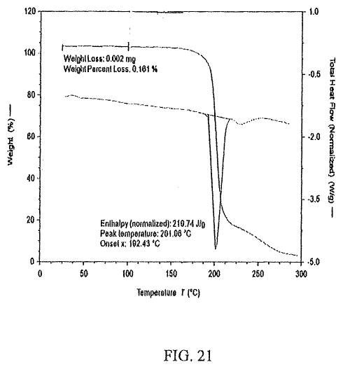

The Mal-1 salt was analyzed using DSC-TGA with the methodology described hereinabove. The data is depicted in FIG. 21. There was a weight loss of about 0.16% by 100 °C. In addition, the DSC thermogram of Mal-1 exhibited one intense peak temperature at about 202.0 °C.

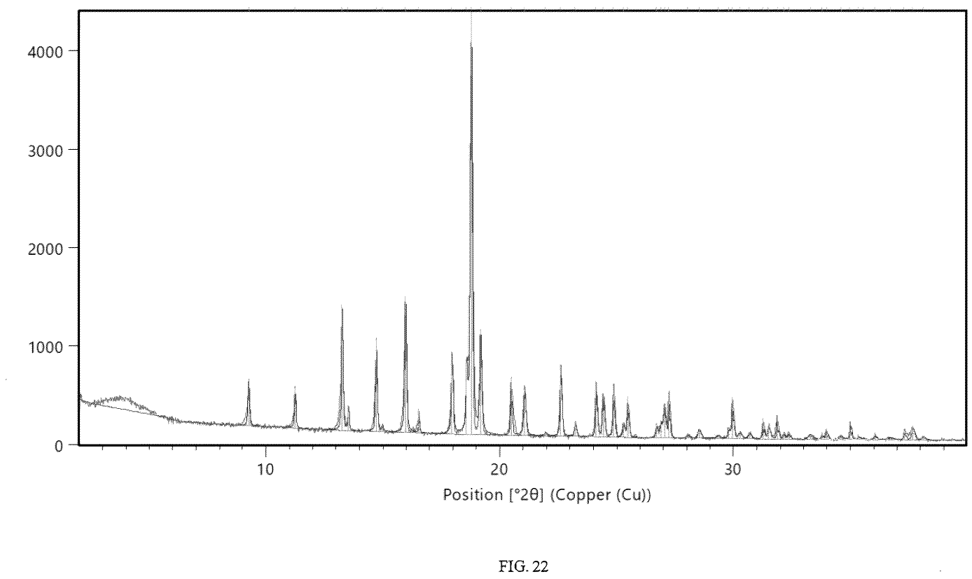

The Mal-2 salt obtained was also analyzed by XRPD. The XRPD diffractogram of Mal-2 is depicted in FIG. 22. The peak values and intensity of the XPRD of the Mal-2 salt are provided below in Table 15.

TABLE 15
XRPD peak values for FIG. 22 (Mal-2).

| Peak # | Pos. [°2θ] | Intensity |
|---|---|---|
| 1 | 9.2553 | 461.76 |
| 2 | 11.2370 | 403.14 |
| 3 | 13.2518 | 1272.25 |
| 4 | 13.5199 | 259.72 |
| 5 | 14.7128 | 927.24 |
| 6 | 14.9777 | 61.96 |
| 7 | 15.9593 | 1385.67 |
| 8 | 16.5301 | 248.15 |
| 9 | 17.9537 | 843.52 |
| 10 | 18.5771 | 757.13 |
| 11 | 18.7771 | 4321.64 |
| 12 | 19.1752 | 1059.71 |
| 13 | 20.4794 | 572.16 |
| 14 | 21.0517 | 464.67 |
| 15 | 21.9470 | 24.63 |
| 16 | 22.6082 | 727.67 |
| 17 | 23.2218 | 162.52 |
| 18 | 24.1143 | 567.37 |
| 19 | 24.4172 | 445.74 |
| 20 | 24.8589 | 518.43 |
| 21 | 25.2863 | 151.91 |
| 22 | 25.4717 | 419.34 |
| 23 | 26.7062 | 130.71 |
| 24 | 26.8939 | 123.75 |
| 25 | 27.0379 | 333.24 |
| 26 | 27.2302 | 450.65 |
| 27 | 28.0588 | 25.50 |
| 28 | 28.5249 | 89.73 |
| 29 | 29.3433 | 29.70 |
| 30 | 29.7942 | 69.58 |
| 31 | 29.9468 | 422.03 |
| 32 | 30.2728 | 68.11 |
| 33 | 30.6787 | 50.73 |
| 34 | 31.2574 | 219.14 |
| 35 | 31.5087 | 132.78 |
| 36 | 31.8671 | 241.68 |
| 37 | 32.1497 | 61.29 |
| 38 | 32.3687 | 69.05 |
| 39 | 33.2868 | 45.66 |
| 40 | 33.7755 | 30.81 |
| 41 | 33.9684 | 112.29 |
| 42 | 34.6040 | 36.98 |
| 43 | 34.9845 | 184.90 |
| 44 | 35.3425 | 27.11 |
| 45 | 35.5566 | 14.53 |
| 46 | 36.0466 | 53.03 |
| 47 | 36.7232 | 19.44 |
| 48 | 37.3013 | 98.14 |
| 49 | 37.6777 | 135.03 |
| 50 | 38.1361 | 34.80 |

As shown, in an embodiment, this Mal-2 salt can be characterized by intense peaks in the X-Ray diffractogram at 13.3, 16.0, and 18.8±0.5 ° 2θ. In another embodiment, this salt can be characterized by peaks at 13.3, 14.7, 16.0, 18.8, and 19.2±0.5 ° 2θ. In another embodiment, this salt can be characterized by peaks at 13.3, 14.7, 16.0, 18.0, 18.6, 18.8, 19.2, 20.5, 22.6, and 24.1±0.5 ° 2θ. In another embodiment, this salt can be characterized by peaks at 9.3, 11.2, 13.3, 13.5, 14.7, 15.0, 16.0, 16.5, 18.0, 18.6, 18.8, 19.2, 20.5, 21.1, 21.9, 22.6, 23.2, 24.1, 24.4, 24.9, 25.3, 25.5, 26.7, 26.9, 27.0, 27.2, 28.1, 28.5, 29.3, 29.8, 29.9, 30.3, 30.7, 31.3, 31.5, 31.9, 32.1, 32.4, 33.3, 33.8, 34.0, 34.6, 35.0, 35.3, 35.6, 36.0, 36.7, 37.3, 37.7, and 38.1±0.5 ° 2θ.

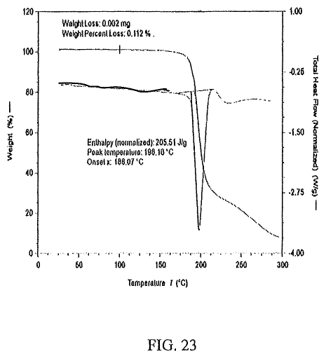

The Mal-2 salt was analyzed using DSC-TGA with the methodology described hereinabove. The data is depicted in FIG. 23. There was a weight loss of about 0.11% by 100 °C. In addition, the DSC thermogram of Mal-2 exhibited one intense peak temperature at about 198.2 °C.

Substituting deuterated R freebase for (R)-2-(4-fluorophenyl)-2-(methylamino)cyclohexan-1-one in the reaction with maleic acid described hereinabove produces two crystalline polymorphs, deuterated (R)-2-(4-fluorophenyl)-2-(methylamino)cyclohexan-1-one Mal-1 (deuterated R maleate-1, also identified herein as deuterated R Mal-1) and deuterated (R)-2-(4-fluorophenyl)-2-(methylamino)cyclohexan-1-one Mal-2 (deuterated R maleate-2, also identified herein as deuterated R Mal-2). In both cases, the stoichiometric ratio of freebase to maleic acid is 1:1. The XRPD and DSC-TGA of deuterated R Mal-1 is substantially the same as the XRPD and DSC-TGA of Mal-1, respectively. In addition, the DVS pattern of deuterated R Mal-1 is substantially the same as that of Mal-1. Furthermore, the XRPD and DSC-TGA of deuterated R Mal-2 is substantially the same as the XRPD and DSC-TGA of Mal-2, respectively. In addition, the DVS pattern of deuterated R Mal-2 is substantially the same as that of Mal-2.

2. (S)-2-(4-fluorophenyl)-2-(methylamino)cyclohexan-1-one maleate and deuterated analog

Substituting S freebase for (R)-2-(4-fluorophenyl)-2-(methylamino)cyclohexan-1-one in the reaction with maleic acid described hereinabove produces two crystalline polymorphs, deuterated(S)-2-(4-fluorophenyl)-2-(methylamino)cyclohexan-1-one Mal-1 (S maleate-1, also identified herein as S Mal-1) and(S)-2-(4-fluorophenyl)-2-(methylamino)cyclohexan-1-one Mal-2 (S maleate-2, also identified herein as S Mal-2). In both cases, the stoichiometric ratio of freebase to maleic acid is 1:1. The XRPD and DSC-TGA of S Mal-1 is substantially the same as the XRPD and DSC-TGA of Mal-1, respectively. In addition, the DVS pattern of S Mal-1 is substantially the same as that of Mal-1. Furthermore, the XRPD and DSC-TGA of S Mal-2 is substantially the same as the XRPD and DSC-TGA of Mal-2, respectively. In addition, the DVS pattern of S Mal-2 is substantially the same as that of Mal-2.

Substituting deuterated S freebase for (R)-2-(4-fluorophenyl)-2-(methylamino)cyclohexan-1-one in the reaction with maleic acid described hereinabove produces two crystalline polymorphs, deuterated(S)-2-(4-fluorophenyl)-2-(methylamino)cyclohexan-1-one Mal-1 (deuterated S maleate-1, also identified herein as deuterated S Mal-1) and deuterated(S)-2-(4-fluorophenyl)-2-(methylamino)cyclohexan-1-one Mal-2 (deuterated S maleate-2, also identified herein as deuterated S Mal-2). In both cases, the stoichiometric ratio of freebase to maleic acid is 1:1. The XRPD and DSC-TGA of deuterated S Mal-1 is substantially the same as the XRPD and DSC-TGA of Mal-1, respectively. In addition, the DVS pattern of deuterated S Mal-1 is substantially the same as that of Mal-1. Furthermore, the XRPD and DSC-TGA of deuterated S Mal-2 is substantially the same as the XRPD and DSC-TGA of Mal-2, respectively. In addition, the DVS pattern of deuterated S Mal-2 is substantially the same as that of Mal-2.

(f) 1. (R)-2-(4-fluorophenyl)-2-(methylamino)cyclohexan-1-one phosphate and deuterated analogs

Isopropanol, acetonitrile, ethyl acetate, tetrahydrofuran, and an acetone/water mixture of 9:1 v/v (0.25 mL of each solvent) were added to five different glass vials each containing about 40 mg of freebase (R)-2-(4-fluorophenyl)-2-(methylamino)cyclohexan-1-one. One molar equivalent of 85 wt % phosphoric acid was added to each vial and the contents were stirred at room temperature. A solid product was observed in each and the solids were collected from each and vacuum dried at room temperature. The products isolated from each of the solvents were crystalline. XRPD analysis showed that two different polymorphs were collected; one polymorph was obtained from isopropanol, tetrahydrofuran, and the acetone/water mixture and is identified as (R)-2-(4-fluorophenyl)-2-(methylamino)cyclohexan-1-one Phos-1 (phosphate-1, also denoted as Phos-1) and another polymorph was obtained from acetonitrile and ethyl acetate and is designated as (R)-2-(4-fluorophenyl)-2-(methylamino)cyclohexan-1-one Phos-2 (phosphate-2, also denoted as Phos-2). The stoichiometric ratio of base to phosphoric acid was not possible to determine by proton NMR.

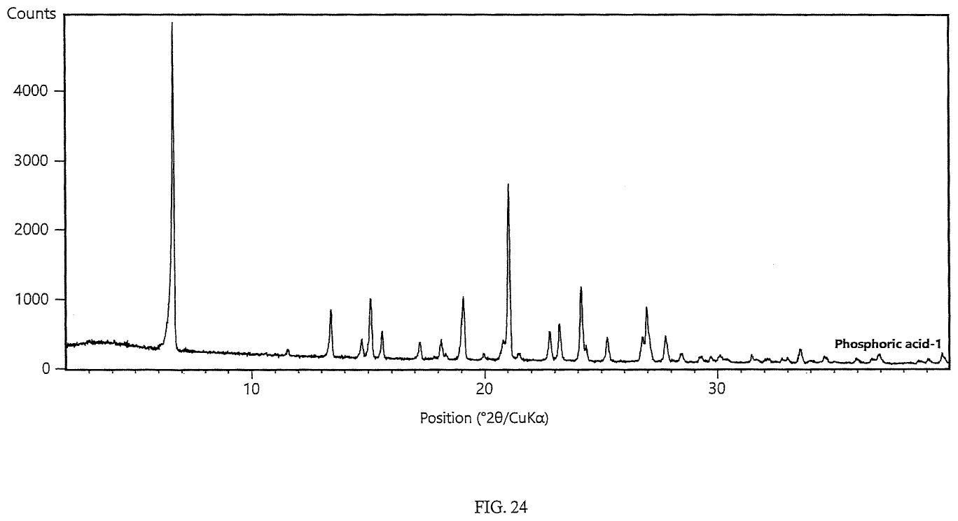

The Phos-1 salt obtained was analyzed by XRPD. The XRPD diffractogram of Phos-1 is depicted in FIG. 24. The peak values and intensity of the XPRD of the Phos-1 salt are provided below in Table 16.

TABLE 16
XRPD peak values for FIG. 24 (Phos-1).

| Peak # | Pos. [°2θ] | Intensity |
|---|---|---|
| 1 | 6.6386 | 4442.59 |
| 2 | 11.5482 | 84.45 |
| 3 | 13.4059 | 654.93 |
| 4 | 14.7275 | 239.08 |
| 5 | 15.1082 | 813.72 |
| 6 | 15.5807 | 357.08 |
| 7 | 17.2142 | 234.76 |
| 8 | 18.1353 | 258.84 |
| 9 | 19.0884 | 818.14 |
| 10 | 19.9530 | 87.21 |
| 11 | 20.7760 | 258.26 |
| 12 | 21.0277 | 2441.43 |
| 13 | 21.4589 | 85.79 |
| 14 | 22.7954 | 402.10 |
| 15 | 23.1959 | 486.71 |
| 16 | 24.1312 | 999.05 |
| 17 | 24.3218 | 149.20 |
| 18 | 25.2499 | 299.86 |
| 19 | 26.7778 | 347.60 |
| 20 | 26.9501 | 758.73 |
| 21 | 27.7621 | 360.05 |
| 22 | 28.4898 | 96.39 |
| 23 | 29.3072 | 74.20 |
| 24 | 29.7067 | 72.73 |
| 25 | 30.0759 | 90.56 |
| 26 | 31.4393 | 103.57 |
| 27 | 31.6326 | 40.81 |
| 28 | 32.1181 | 47.37 |
| 29 | 32.7531 | 62.83 |
| 30 | 32.9968 | 63.61 |
| 31 | 33.5429 | 191.26 |
| 32 | 33.9549 | 31.31 |
| 33 | 34.5658 | 91.37 |
| 34 | 35.9617 | 59.11 |
| 35 | 36.6269 | 59.53 |
| 36 | 36.9259 | 116.94 |
| 37 | 38.6750 | 32.79 |
| 38 | 39.0339 | 59.97 |
| 39 | 39.6387 | 143.88 |

As shown, in an embodiment, this Phos-1 salt can be characterized by peaks in the X-ray diffractogram at 6.6, 19.1, 21.0, and 24.1±0.5 ° 2θ. In another embodiment, this salt can be characterized by peaks at 6.6, 15.1, 19.1, 21.0, and 24.1±0.5 ° 2θ. In another embodiment, this salt can be characterized by peaks at 6.6, 13.4, 15.1, 19.1, 21.0, 22.8, 23.2, 24.1, 27.0, and 27.8±0.5 ° 2θ. In another embodiment, this salt can be characterized by peaks at 6.6, 11.5, 13.4, 14.7, 15.1, 15.6, 17.2, 18.1, 19.1, 20.0, 20.8, 21.0, 21.5, 22.8, 23.2, 24.1, 24.3, 25.2, 26.8, 27.0, 27.8, 28.5, 29.3, 29.7, 30.1, 31.4, 31.6, 32.1, 32.8, 33.0, 33.5, 34.0, 34.6, 36.0, 36.6, 36.9, 38.7, 39.0, and 39.6±0.5 ° 2θ.

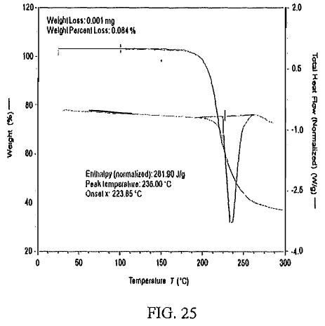

The Phos-1 salt was analyzed using DSC-TGA with the methodology described hereinabove. The data is depicted in FIG. 25. There was a weight loss of about 0.11% by 100 °C. In addition, the DSC thermogram of Phos-1 exhibited one intense peak temperature at about 236.0 °C.

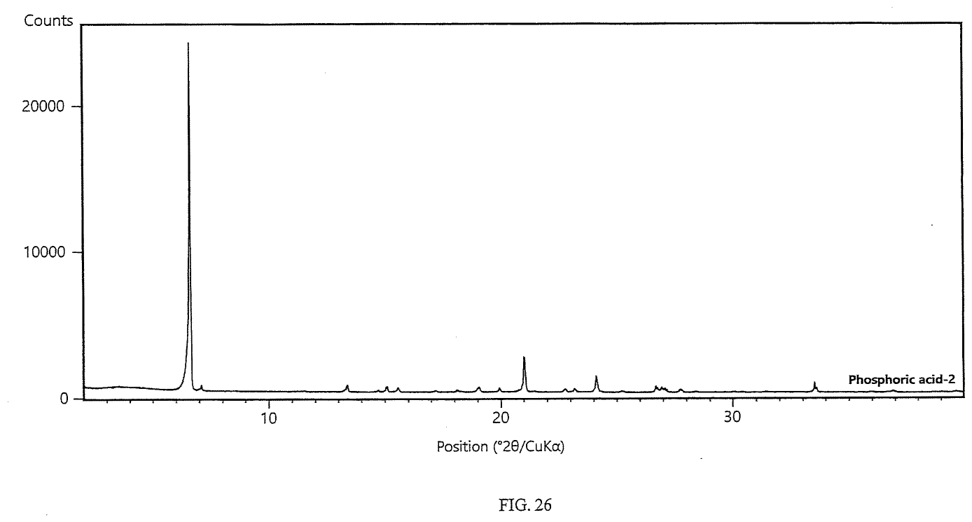

The Phos-2 salt obtained was analyzed by XRPD. The XRPD diffractogram of Phos-2 is depicted in FIG. 26. The peak values and intensity of the XPRD of the Phos-2 salt are provided below in Table 17.

TABLE 17
XRPD peak values for FIG. 26 (Phos-2).

| Peak # | Pos. [°2θ] | Intensity |
|---|---|---|
| 1 | 3.5187 | 92.94 |
| 2 | 6.6209 | 22232.20 |
| 3 | 7.0879 | 338.85 |
| 4 | 13.3764 | 441.45 |
| 5 | 14.7087 | 107.25 |
| 6 | 15.0942 | 288.93 |
| 7 | 15.5558 | 258.22 |
| 8 | 17.1973 | 86.73 |
| 9 | 18.1044 | 119.76 |
| 10 | 19.0564 | 291.86 |
| 11 | 19.9230 | 273.00 |
| 12 | 21.0023 | 2255.89 |
| 13 | 22.7472 | 216.14 |
| 14 | 23.1739 | 219.25 |
| 15 | 24.1072 | 1012.72 |
| 16 | 25.2430 | 79.85 |
| 17 | 26.6723 | 352.86 |
| 18 | 26.7486 | 299.25 |
| 19 | 26.9246 | 318.84 |
| 20 | 27.0934 | 168.28 |
| 21 | 27.7341 | 189.27 |
| 22 | 28.3871 | 35.58 |
| 23 | 29.2432 | 19.19 |
| 24 | 29.6744 | 21.06 |
| 25 | 30.0561 | 41.57 |
| 26 | 30.3712 | 31.38 |
| 27 | 31.4294 | 51.65 |
| 28 | 31.6452 | 15.52 |
| 29 | 33.5128 | 598.48 |
| 30 | 34.5961 | 22.83 |
| 31 | 35.9402 | 21.94 |
| 32 | 36.9090 | 87.29 |
| 33 | 39.6273 | 56.03 |

As shown, in an embodiment, this Phos-2 salt can be characterized by peaks in the X-Ray diffractogram at 6.6, 21.0, 24.1, and 33.5±0.5 ° 2θ. In another embodiment, this salt can be characterized by peaks at 6.6, 13.4, 21.0, 24.1, and 33.5±0.5 ° 2θ. In another embodiment, this salt can be characterized by peaks at 6.6, 7.1, 13.4, 19.1, 21.0, 24.1, 26.7 (26.67), 26.7 (26.75), 26.9, and 33.5±0.5 ° 2θ. In another embodiment, this salt can be characterized by peaks at 3.5, 6.6, 7.1, 13.4, 14.7, 15.1, 15.6, 17.2, 18.1, 19.1, 19.9, 21.0, 22.7, 23.2, 24.1, 25.2, 26.7 (26.67), 26.7 (26.75), 26.9, 27.1, 27.7, 28.4, 29.2, 29.7, 30.1, 30.4, 31.4, 31.6, 33.5, 34.6, 35.9, 36.9, and 39.6±0.5 ° 2θ.

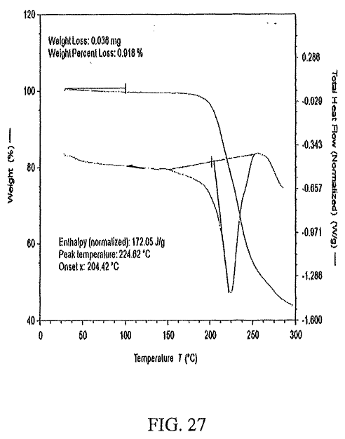

The Phos-2 salt was analyzed using DSC-TGA with the methodology described hereinabove. The data is depicted in FIG. 27. There was a weight loss of about 0.90% by 100 °C. In addition, the DSC thermogram of Phos-2 exhibited one broad peak temperature at about 224.6 °C.

Substituting deuterated R freebase for freebase (R)-2-(4-fluorophenyl)-2-(methylamino)cyclohexan-1-one in the various reactions with 85 wt % phosphoric acid, as described above produces two crystalline polymorph salts, deuterated (R)-2-(4-fluorophenyl)-2-(methylamino)cyclohexan-1-one phosphate-1 (deuterated R phosphate-1, also identified as deuterated R Phos-1) and deuterated (R)-2-(4-fluorophenyl)-2-(methylamino)cyclohexan-1-one phosphate-2 (deuterated R phosphate-2, also identified as deuterated R Phos-2). The XRPD diffractogram and DSC-TGA of deuterated R Phos-1 is substantially the same as the XRPD diffractogram and DSC-TGA of Phos-1. The DVS pattern of deuterated R phosphate-1 is substantially the same as that of Phos-1. The XRPD diffractogram and DSC-TGA of deuterated R Phos-2 is substantially the same as the XRPD diffractogram and DSC-TGA of Phos-2. In addition, the DVS pattern of deuterated R Phos-2 is substantially the same as that of Phos-2.

2. (S)-2-(4-fluorophenyl)-2-(methylamino)cyclohexan-1-one phosphate and deuterated analogs

Substituting S freebase for freebase (R)-2-(4-fluorophenyl)-2-(methylamino)cyclohexan-1-one in the various reactions with 85 wt % phosphoric acid, as described above produces two crystalline polymorph salts, (S)-2-(4-fluorophenyl)-2-(methylamino)cyclohexan-1-one phosphate-1 (S phosphate-1, also identified as S Phos-1) and(S)-2-(4-fluorophenyl)-2-(methylamino)cyclohexan-1-one phosphate-2 (S phosphate-2, also identified as S Phos-2). The XRPD diffractogram and DSC-TGA of S Phos-1 is substantially the same as the XRPD diffractogram and DSC-TGA of Phos-1. The DVS pattern of S Phos-1 is substantially the same as that of Phos-1. The XRPD diffractogram and DSC-TGA of S Phos-2 is substantially the same as the XRPD diffractogram and DSC-TGA of Phos-2. In addition, the DVS pattern of S Phos-2 is substantially the same as that of Phos-2.

Substituting deuterated S freebase for freebase (R)-2-(4-fluorophenyl)-2-(methylamino)cyclohexan-1-one in the various reactions with 85 wt % phosphoric acid, as described above, produces two crystalline polymorph salts, deuterated(S)-2-(4-fluorophenyl)-2-(methylamino)cyclohexan-1-one phosphate-1 (deuterated S phosphate-1, also identified as deuterated S Phos-1) and deuterated (R)-2-(4-fluorophenyl)-2-(methylamino)cyclohexan-1-one phosphate-2 (deuterated S phosphate-2, also identified as deuterated S Phos-2). The XRPD diffractogram and DSC-TGA of deuterated S Phos-1 is substantially the same as the XRPD diffractogram and DSC-TGA of Phos-1. The DSC thermogram of deuterated S Phos-1 exhibits one intense peak temperature at about 236.0 °C. The XRPD diffractogram and DSC-TGA of deuterated S Phos-2 is substantially the same as the XRPD diffractogram and DSC-TGA of Phos-2. In addition, the DVS pattern of deuterated S Phos-2 is substantially the same as that of Phos-2.

(g) (R)-2-(4-fluorophenyl)-2-(methylamino)cyclohexan-1-one L-tartrate and deuterated analogs thereof

Isopropanol, ethyl acetate, tetrahydrofuran, and an acetone/water mixture of 9:1 (v/v) (0.25 mL of each solvent) were added to four different glass vials each containing about 40 mg of freebase (R)-2-(4-fluorophenyl)-2-(methylamino)cyclohexan-1-one. One molar equivalent of L-tartaric acid was added to each vial, and the contents were stirred at room temperature. A solid product was observed in each and was collected from each and vacuum dried at room temperature. The products isolated from each of the solvents were crystalline. As determined by XRPD analysis, six different crystalline products were collected. One product was obtained from isopropanol and will be identified as (R)-2-(4-fluorophenyl)-2-(methylamino)cyclohexan-1-one L-Tar-1 (tartrate-1, also designated as Tar-1). Two products were isolated from ethyl acetate and will be identified as (R)-2-(4-fluorophenyl)-2-(methylamino)cyclohexan-1-one Tar-2 (tartrate-2, also identified as Tar-2) and (R)-2-(4-fluorophenyl)-2-(methylamino)cyclohexan-1-one Tar-5 (tartrate-5, also identified as Tar-5). As discussed below, Tar-2 converts to Tar-5. A fourth product was isolated from tetrahydrofuran and will be identified as (R)-2-(4-fluorophenyl)-2-(methylamino)cyclohexan-1-one Tar-3 (tartrate-3, also designated as Tar-3). Finally, two products were isolated from the acetone/water mixture and will be identified as (R)-2-(4-fluorophenyl)-2-(methylamino)cyclohexan-1-one Tar-4 (tartrate-4, also identified as Tar-4) and (R)-2-(4-fluorophenyl)-2-(methylamino)cyclohexan-1-one Tar-6 (tartrate-6, also identified as Tar-6). As explained below, Tar-4 converts to Tar-6. Except for Tar 2 and Tar-5, which had an acid to free base ratio of 1.6:1, and Tar-3 which was a hemitartrate, the other polymorphs were 1:1 salts by NMR analysis.

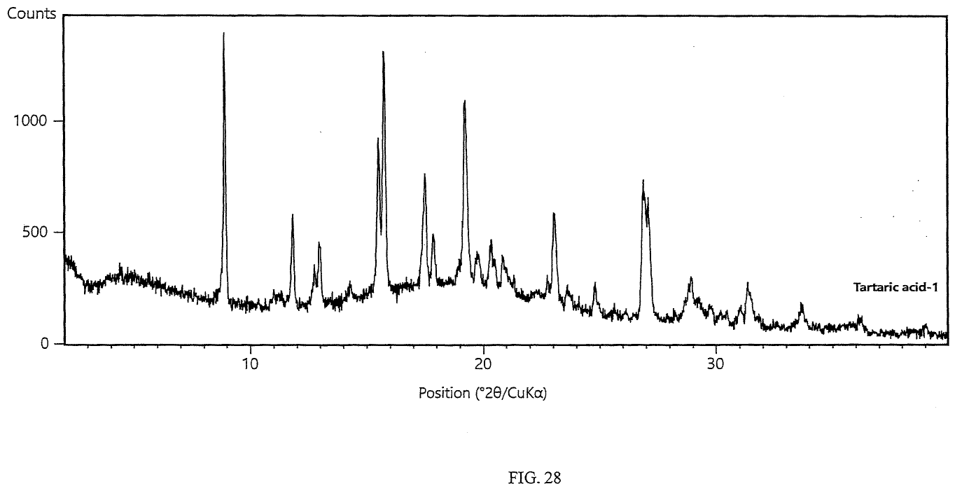

Tar-1 was isolated from isopropanol and is crystalline. The Tar-1 salt obtained was analyzed by XRPD. The XRPD diffractogram of Tar-1 is depicted in FIG. 28. The peak values and intensity of the XPRD of the Tar-1 salt are provided below in Table 18.

TABLE 18
XRPD peak values for FIG. 28 (Tar-1).

| Peak # | Pos. [°2θ] | Intensity |
|---|---|---|
| 1 | 8.8965 | 1204.75 |
| 2 | 11.1291 | 42.39 |
| 3 | 11.8244 | 420.46 |
| 4 | 12.3506 | 27.04 |
| 5 | 12.7453 | 126.01 |
| 6 | 12.9768 | 288.46 |
| 7 | 14.2488 | 97.64 |
| 8 | 15.5022 | 776.09 |
| 9 | 15.7438 | 1159.01 |
| 10 | 17.5066 | 596.01 |
| 11 | 17.8461 | 346.99 |
| 12 | 19.2063 | 957.63 |
| 13 | 19.7532 | 269.06 |
| 14 | 20.3499 | 345.92 |
| 15 | 20.8264 | 267.13 |
| 16 | 23.0449 | 486.50 |
| 17 | 23.5694 | 120.33 |
| 18 | 24.7990 | 159.59 |
| 19 | 25.6081 | 28.03 |
| 20 | 26.1047 | 32.39 |
| 21 | 26.8585 | 577.59 |
| 22 | 27.1001 | 517.29 |
| 23 | 28.6658 | 93.17 |
| 24 | 28.9456 | 211.34 |
| 25 | 29.2577 | 91.26 |
| 26 | 29.7721 | 84.13 |
| 27 | 30.1555 | 47.90 |
| 28 | 30.4372 | 49.17 |
| 29 | 31.0068 | 80.28 |
| 30 | 31.3340 | 188.41 |
| 31 | 33.6852 | 94.81 |
| 32 | 36.2154 | 50.26 |

As shown, in an embodiment, this Tar-1 salt can be characterized by peaks in the X-Ray diffractogram at 8.9, 15.7, and 19.2±0.5 ° 2θ. In another embodiment, this salt can be characterized by peaks at 8.9, 15.5, 15.7, 17.5, and 19.2±0.5 ° 2θ. In another embodiment, this salt can be characterized by peaks at 8.9, 11.8, 15.5, 15.7, 17.5, 17.8, 19.2, 23.0, 26.9, and 27.1±0.5 ° 2θ. In another embodiment, this salt can be characterized by peaks at 8.9, 11.1, 11.8, 12.4, 12.7, 13.0, 14.2, 15.5, 15.7, 17.5, 17.8, 19.2, 19.8, 20.3, 20.8, 23.0, 23.6, 24.8, 25.6, 26.1, 26.9, 27.1, 28.7, 28.9, 29.3, 29.8, 30.2, 30.4, 31.0, 31.3, 33.7, and 36.2±0.5 ° 2θ.

As indicated above, Tar-2 and Tar-5 were obtained from the reaction of freebase with L-tartaric acid in ethyl acetate. Both Tar-2 and Tar-5 are crystalline forms. Tar-2 was the initial crystal that exists in the wet cake from filtration. Upon drying in a vacuum oven at room temperature, Tar-2 transitioned into Tar-5. As determined by proton NMR, the stoichiometric ratio of base to acid of Tar-2 and Tar-5 was 1:1.6.

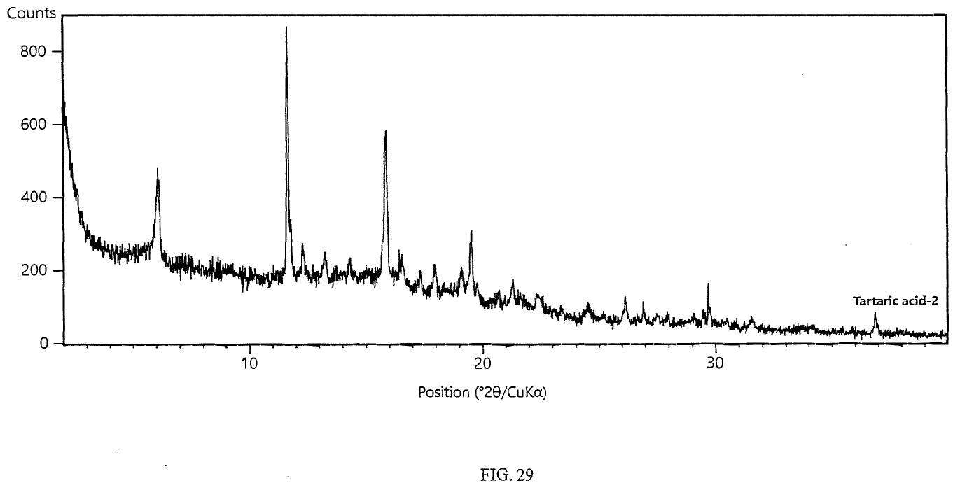

The Tar-2 salt obtained was analyzed by XRPD. The XRPD diffractogram of Tar-2 is depicted in FIG. 29. The peak values and intensity of the XPRD of the Tar-2 salt are provided below in Table 19.

TABLE 19
XRPD peak values for FIG. 29 (Tar-2).

| Peak # | Pos. [°2θ] | Intensity |
|---|---|---|
| 1 | 6.0785 | 252.38 |
| 2 | 11.6290 | 702.82 |
| 3 | 11.7515 | 120.92 |
| 4 | 12.2956 | 107.05 |
| 5 | 13.2298 | 87.62 |
| 6 | 14.2661 | 41.65 |
| 7 | 15.8517 | 455.11 |
| 8 | 16.5156 | 99.27 |
| 9 | 17.2952 | 56.64 |
| 10 | 17.9505 | 92.57 |
| 11 | 19.0770 | 91.63 |
| 12 | 19.5051 | 203.01 |
| 13 | 21.2799 | 77.30 |
| 14 | 22.3505 | 52.98 |
| 15 | 24.5158 | 30.16 |
| 16 | 26.1215 | 61.06 |
| 17 | 26.8920 | 33.65 |
| 18 | 27.4691 | 16.64 |
| 19 | 27.9000 | 15.51 |
| 20 | 29.0468 | 19.60 |
| 21 | 29.4672 | 42.62 |
| 22 | 29.6774 | 111.60 |
| 23 | 31.5349 | 24.03 |
| 24 | 33.9501 | 10.07 |
| 25 | 36.8307 | 54.74 |

As shown, in an embodiment, this Tar-2 salt can be characterized by peaks in the X-Ray diffractogram at 6.1, 11.6, and 15.9±0.5 ° 2θ. In another embodiment, this salt can be characterized by peaks at 6.1, 11.6, 11.8, 15.9, and 19.5±0.5 ° 2θ. In another embodiment, this salt can be characterized by peaks at 6.1, 11.6, 11.8, 12.3, 15.9, 16.5, 18.0, 19.1, 19.5, and 29.7±0.5 ° 2θ. In another embodiment, this salt can be characterized by peaks at 6.1, 11.6, 11.8, 12.3, 13.2, 14.3, 15.9, 16.5, 17.3, 18.0, 19.1, 19.5, 21.3, 22.4, 24.5, 26.1, 26.9, 27.5, 27.9, 26.9, 29.0, 29.5, 29.7, 31.5, 34.0, and 36.8±0.5 ° 2θ.

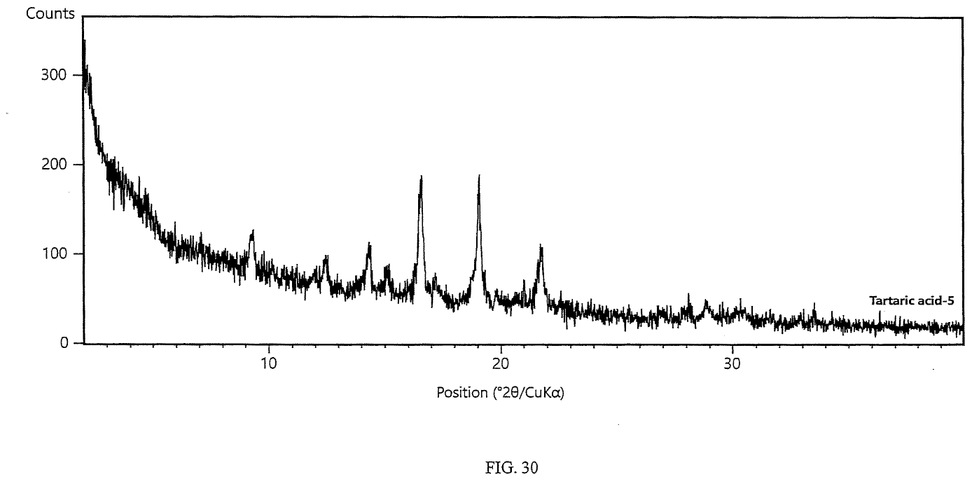

Tar-5, which is the product to which Tar-2 converts upon drying, was analyzed by XRPD. The XRPD diffractogram of Tar-5 is depicted in FIG. 30. The peak values and intensity of the XPRD of the Tar-5 salt are provided below in Table 20.

TABLE 20
XRPD peak values for FIG. 30 (Tar-5).

| Peak # | Pos. [°2θ] | Intensity |
|---|---|---|
| 1 | 9.3116 | 41.07 |
| 2 | 11.9824 | 10.08 |
| 3 | 12.4447 | 29.79 |
| 4 | 14.3398 | 46.90 |
| 5 | 15.0976 | 24.16 |
| 6 | 16.5683 | 128.28 |
| 7 | 19.0470 | 135.60 |
| 8 | 21.7262 | 64.67 |
| 9 | 28.8991 | 12.57 |

As shown, in an embodiment, this Tar-5 salt can be characterized by peaks in the X-Ray diffractogram at 16.6, 19.0, and 21.7±0.5 ° 2θ. In another embodiment, this salt can be characterized by peaks at 9.3, 14.3, 16.6, 19.0, and 21.7±0.5 ° 2θ. In another embodiment, this salt can be characterized by peaks at 9.3, 12.0, 12.4, 14.3, 15.1, 16.6, 19.0, 21.7, and 28.9±0.5 ° 2θ.

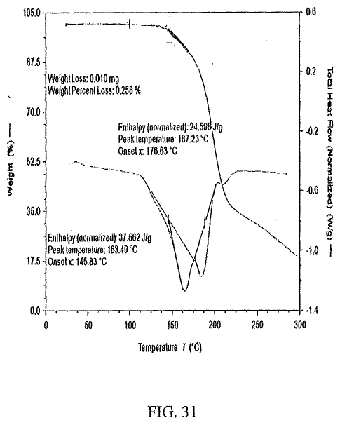

The Tar-5 salt was analyzed by DSC-TGA using the methodology described hereinabove. The data is depicted in FIG. 31. There was a weight loss of about 0.26% by 100 °C. In addition, the DSC thermogram of the Tar-5 salt exhibited two broad peak temperatures at about 163.5 and about 187.2 °C.

As indicated above, Tar-3 was isolated from the reaction of freebase and L-tartaric acid in THF. The Tar-3 salt is a hemitartrate, as the stoichiometric ratio of base: acid was 1.0:0.5, as determined by proton NMR. Also, noted in the NMR spectrum of Tar-3 crystals was the presence of THF solvent.

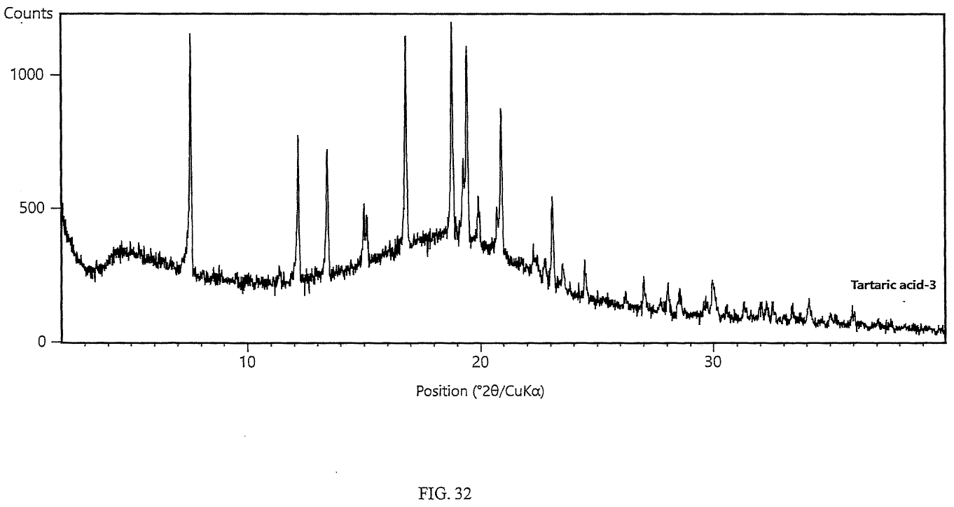

The Tar-3 obtained was analyzed by XRPD. The XRPD diffractogram of Tar-3 is depicted in FIG. 32. The peak values and intensity of the XPRD of the Tar-3 salt are provided below in Table 21.

TABLE 21
XRPD peak values for FIG. 32 (Tar-3).

| Peak # | Pos. [°2θ] | Intensity |
|---|---|---|
| 1 | 7.5630 | 867.18 |
| 2 | 12.1828 | 543.03 |
| 3 | 13.4211 | 506.15 |
| 4 | 14.9936 | 318.17 |
| 5 | 16.7966 | 953.75 |
| 6 | 18.7729 | 1013.10 |
| 7 | 19.2377 | 487.94 |
| 8 | 19.4118 | 925.01 |
| 9 | 19.9152 | 350.70 |
| 10 | 20.7353 | 108.32 |
| 11 | 20.8835 | 705.82 |
| 12 | 22.3076 | 171.20 |
| 13 | 22.7566 | 102.10 |
| 14 | 23.0713 | 371.78 |
| 15 | 23.5370 | 140.86 |
| 16 | 24.4799 | 158.57 |
| 17 | 26.2198 | 38.19 |
| 18 | 26.9908 | 108.74 |
| 19 | 28.0381 | 97.92 |
| 20 | 28.5574 | 74.21 |
| 21 | 29.6378 | 30.45 |
| 22 | 29.9618 | 125.54 |
| 23 | 31.3249 | 42.15 |
| 24 | 32.0024 | 44.22 |
| 25 | 32.2733 | 51.55 |
| 26 | 32.2948 | 31.30 |
| 27 | 32.5078 | 43.99 |
| 28 | 33.3784 | 41.73 |
| 29 | 34.0762 | 71.89 |
| 30 | 35.9818 | 37.03 |

As shown, in an embodiment, this Tar-3 salt can be characterized by peaks in the X-Ray diffractogram at 16.8, 18.8, and 19.4±0.5 ° 2θ. In another embodiment, this salt can be characterized by peaks at 7.6, 16.8, 18.8, 19.4, and 20.9±0.5 ° 2θ. In another embodiment, this salt can be characterized by peaks at 7.6, 12.2, 13.4, 16.8, 18.8, 19.2, 19.4, 19.9, 20.9, and 23.1±0.5 ° 2θ. In another embodiment, this salt can be characterized by peaks at 7.6, 12.2, 13.4, 15.0, 16.8, 18.8, 19.2, 19.4, 19.9, 20.7, 20.9, 22.3, 22.8, 23.1, 23.5, 24.5, 26.2, 27.0, 28.0, 28.6, 29.6, 30.0, 31.3, 32.0, 32.3 (32.27), 32.3 (32.29), 32.5, 33.4, 34.1, and 36.0±0.5 ° 2θ.

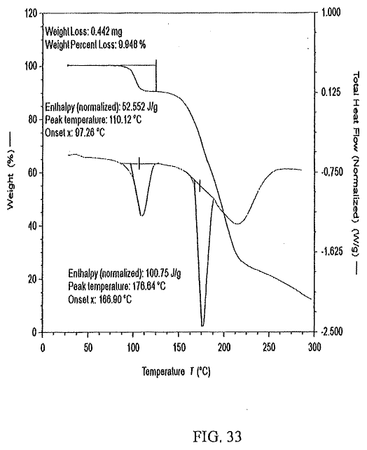

The Tar-3 salt was analyzed by DSC-TGA using the methodology described hereinabove. The data is depicted in FIG. 33. There was a weight loss of about 9.95% when the temperature was raised from room temperature to 125 °C. In addition, the DSC thermogram of the Tar-3 salt exhibited two peak temperatures, a sharp peak at about 176.6 °C and a broader and significantly smaller peak at about 110.1 °C.

As indicated above, Tar-4 and Tar-6 were obtained from the reaction of freebase with L-tartaric acid in a mixture of acetone/water in a 9:1 ratio (v/v). Tar-4 was the crystal form in the wet cake from filtration and was converted into Tar-6 upon drying in a vacuum oven at room temperature. Both Tar 4 and Tar-6 were found to be crystalline. As determined by proton NMR, the stoichiometric ratio of freebase to acid of Tar-6 was 1:1.

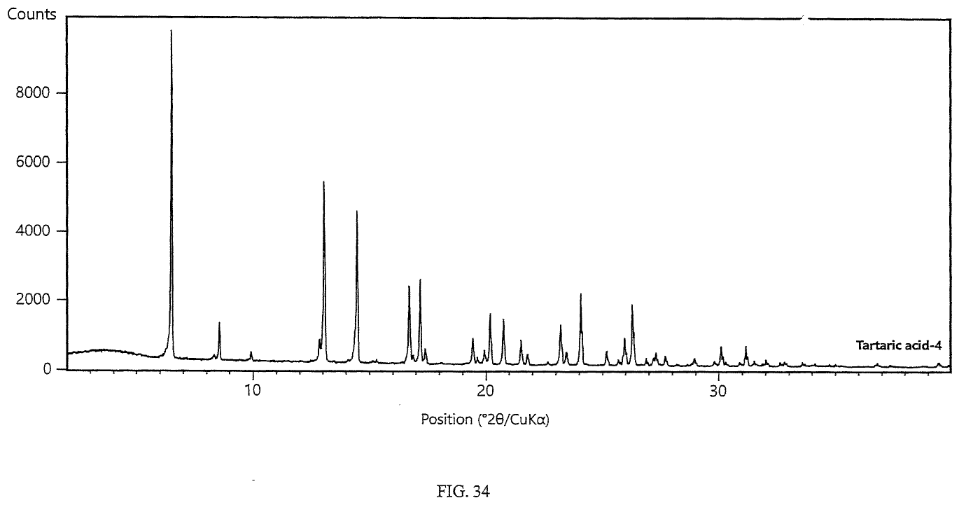

The Tar-4 salt obtained was analyzed by XRPD. The XRPD diffractogram of Tar-4 is depicted in FIG. 34. The peak values and intensity of the XPRD of Tar-4 salt are provided below in Table 22.

TABLE 22
XRPD peak values for FIG. 34 (Tar-4).

| Peak # | Pos. [°2θ] | Intensity |
|---|---|---|
| 1 | 6.5058 | 9249.33 |
| 2 | 8.3075 | 113.47 |
| 3 | 8.5506 | 1045.61 |
| 4 | 9.9120 | 247.89 |
| 5 | 12.8479 | 644.15 |
| 6 | 13.0470 | 5111.05 |
| 7 | 14.4629 | 4297.89 |
| 8 | 15.3086 | 62.05 |
| 9 | 16.7005 | 2216.35 |
| 10 | 16.8566 | 226.74 |
| 11 | 17.1596 | 2369.47 |
| 12 | 17.3936 | 425.82 |
| 13 | 19.4248 | 735.32 |
| 14 | 19.6235 | 189.28 |
| 15 | 19.9214 | 401.13 |
| 16 | 20.1720 | 1395.72 |
| 17 | 20.7392 | 1279.05 |
| 18 | 21.4973 | 706.84 |
| 19 | 21.7700 | 307.66 |
| 20 | 22.6663 | 74.07 |
| 21 | 23.1997 | 1151.84 |
| 22 | 23.4493 | 345.47 |
| 23 | 24.0627 | 1999.37 |
| 24 | 25.1771 | 391.70 |
| 25 | 25.6899 | 154.21 |
| 26 | 25.9534 | 781.38 |
| 27 | 26.2746 | 1691.86 |
| 28 | 26.3545 | 905.14 |
| 29 | 26.8796 | 193.60 |
| 30 | 27.1902 | 194.82 |
| 31 | 27.2985 | 373.29 |
| 32 | 27.6973 | 272.40 |
| 33 | 28.2061 | 26.31 |
| 34 | 28.9556 | 216.91 |
| 35 | 29.8074 | 133.69 |
| 36 | 30.0939 | 567.26 |
| 37 | 30.2660 | 87.79 |
| 38 | 30.8926 | 105.59 |
| 39 | 31.1510 | 559.25 |
| 40 | 31.2369 | 252.62 |
| 41 | 31.5112 | 132.40 |
| 42 | 31.8663 | 51.58 |
| 43 | 32.0219 | 176.25 |
| 44 | 32.6190 | 109.45 |
| 45 | 32.8287 | 105.45 |
| 46 | 33.5786 | 102.32 |
| 47 | 34.1238 | 87.58 |
| 48 | 35.1511 | 21.89 |
| 49 | 36.8056 | 99.53 |
| 50 | 37.4013 | 20.60 |
| 51 | 39.4354 | 119.15 |

As shown, in an embodiment, this Tar-4 salt can be characterized by peaks in the X-Ray diffractogram at 6.5, 13.0, and 14.5±0.5 ° 2θ. In another embodiment, this salt can be characterized by peaks at 6.5, 13.0, 14.5, 16.7, and 17.2±0.5 ° 2θ. In another embodiment, this salt can be characterized by peaks at 6.5, 8.6, 13.0, 14.5, 16.7, 17.2, 20.2, 20.7, 24.1, and 26.3±0.5 ° 2θ. In another embodiment, this salt can be characterized by peaks at 6.5, 8.3, 8.6, 9.9, 12.8, 13.0, 14.5, 15.3, 16.7, 16.9, 17.2, 17.4, 19.4, 19.6, 19.9, 20.2, 20.7, 21.5, 21.8, 22.7, 23.2, 23.4, 24.1, 25.2, 25.7, 26.0, 26.3, 26.4, 26.9, 27.2, 27.3, 27.7, 28.2, 29.0, 29.8, 30.1, 30.3, 30.9, 31.2 (31.15), 31.2 (31.24), 31.5, 31.9, 32.0, 32.6, 32.8, 33.6, 34.1, 35.2, 36.8, 37.4, 39.4±0.5 ° 2θ.

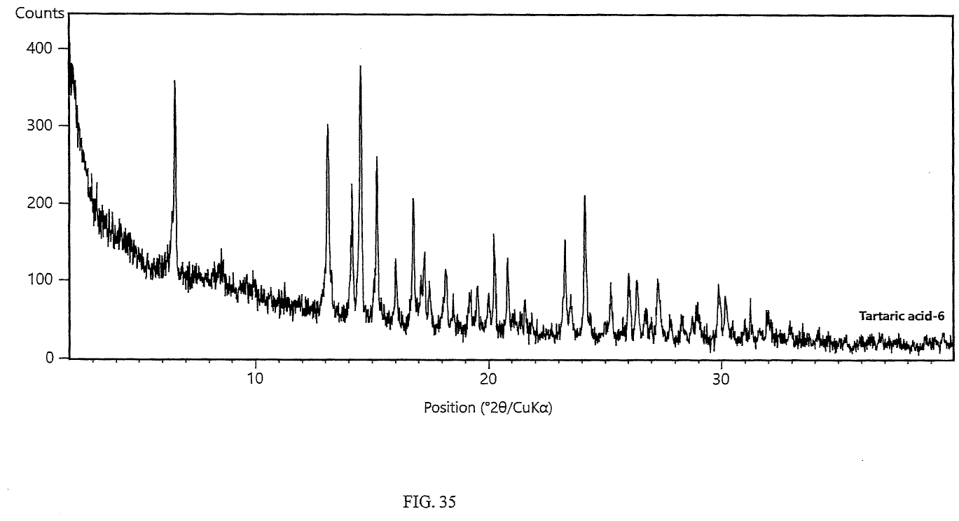

Tar-6, which is the product to which Tar-4 converts, was analyzed by XRPD. The XRPD diffractogram of Tar-6 is depicted in FIG. 35. The peak values and intensity of the XPRD of the Tar-6 salt are provided below in Table 23.

TABLE 23
XRPD peak values for FIG. 35 (Tar-6).

| Peak # | Pos. [°2θ] | Intensity |
|---|---|---|
| 1 | 6.4283 | 47.19 |
| 2 | 6.5560 | 254.47 |
| 3 | 13.0946 | 237.92 |
| 4 | 14.1515 | 153.37 |
| 5 | 14.5367 | 306.83 |
| 6 | 15.2000 | 196.30 |
| 7 | 16.0287 | 67.32 |
| 8 | 16.7749 | 158.14 |
| 9 | 17.0962 | 32.65 |
| 10 | 17.2382 | 90.88 |
| 11 | 17.4616 | 47.73 |
| 12 | 18.1697 | 72.26 |
| 13 | 18.4582 | 19.31 |
| 14 | 19.2051 | 37.05 |
| 15 | 19.5147 | 52.96 |
| 16 | 19.9766 | 25.99 |
| 17 | 20.2305 | 111.75 |
| 18 | 20.8001 | 91.82 |
| 19 | 21.0783 | 15.81 |
| 20 | 21.3630 | 13.85 |
| 21 | 21.5568 | 37.36 |
| 22 | 21.8319 | 17.43 |
| 23 | 23.2815 | 129.52 |
| 24 | 23.5098 | 31.80 |
| 25 | 24.1307 | 186.45 |
| 26 | 25.2487 | 62.43 |
| 27 | 26.0262 | 72.59 |
| 28 | 26.3613 | 70.45 |
| 29 | 26.7127 | 22.51 |
| 30 | 26.9751 | 15.65 |
| 31 | 27.2622 | 75.92 |
| 32 | 27.8008 | 17.14 |
| 33 | 28.3020 | 25.86 |
| 34 | 28.7152 | 15.13 |
| 35 | 28.9772 | 34.87 |
| 36 | 29.8602 | 67.31 |
| 37 | 30.1716 | 54.81 |
| 38 | 31.2197 | 45.35 |
| 39 | 31.5938 | 10.74 |
| 40 | 31.9903 | 28.98 |
| 41 | 32.9412 | 17.37 |

As shown, in an embodiment, this Tar-6 salt can be characterized by peaks in the X-Ray diffractogram at 6.5, 13.1, and 14.5±0.5 ° 2θ. In another embodiment, this salt can be characterized by peaks at 6.5, 13.1, 14.5, 15.2, and 24.1±0.5 ° 2θ. In another embodiment, this salt can be characterized by peaks at 6.6, 13.1, 14.2, 14.5, 15.2, 16.8, 20.2, 20.8, 23.3, and 24.1±0.5 ° 2θ. In another embodiment, this salt can be characterized by peaks at 6.4, 6.6, 13.1, 14.2, 14.5, 15.2, 16.0, 16.8, 17.1, 17.2, 17.5, 18.1, 18.5, 19.2, 19.5, 20.0, 20.2, 20.8, 21.1, 21.4, 21.6, 21.8, 23.3, 23.5, 24.1, 25.2, 26.0, 26.3, 26.7, 27.0, 27.3, 27.8, 28.3, 28.7, 29.0, 29.9, 30.2, 31.2, 31.6, 32.0, and 32.9±0.5 ° 2θ.

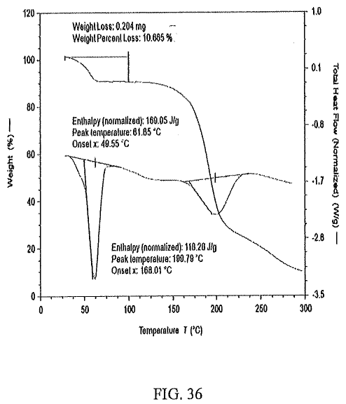

The Tar-6 salt was analyzed by DSC-TGA using the methodology described hereinabove. The data is depicted in FIG. 36. There was a weight loss of about 10.67% by 100 °C. In addition, the DSC thermogram of the Tar-6 salt exhibited two peak temperatures, a peak at about 61.7 °C and a broader and significantly smaller peak at about 199.8 °C.

Substituting deuterated R freebase for freebase (R)-2-(4-fluorophenyl)-2-(methylamino)cyclohexan-1-one in the various solvents of isopropanol, ethyl acetate, tetrahydrofuran, and an acetone/water mixture of 9:1 (v/v), as described hereinabove, six crystalline polymorph salts are produced. One product is obtained from isopropanol and will be identified as deuterated (R)-2-(4-fluorophenyl)-2-(methylamino)cyclohexan-1-one-L-Tar-1 (deuterated R tartrate-1 also designated as deuterated R Tar-1). Two products are isolated from ethyl acetate and will be identified as deuterated (R)-2-(4-fluorophenyl)-2-(methylamino)cyclohexan-1-one-L-Tar-2 (deuterated R tartrate-2 also designated as deuterated R Tar-2) and deuterated (R)-2-(4-fluorophenyl)-2-(methylamino)cyclohexan-1-one-L-Tar-5 (deuterated R tartrate-5 also designated as deuterated R Tar-5). As discussed below, deuterated R Tar-2 converts to deuterated R tartrate-5. A fourth product is isolated from tetrahydrofuran and will be identified as deuterated (R)-2-(4-fluorophenyl)-2-(methylamino)cyclohexan-1-one-L-Tar-3 (deuterated R tartrate-3 also designated as deuterated R Tar-3). Finally, two products are isolated from the acetone/water mixture and will be identified as deuterated (R)-2-(4-fluorophenyl)-2-(methylamino)cyclohexan-1-one-L-Tar-4 (deuterated R tartrate-4 also designated as deuterated R Tar-4) and deuterated (R)-2-(4-fluorophenyl)-2-(methylamino)cyclohexan-1-one-L-Tar-6 (deuterated R tartrate-6 also designated as deuterated R Tar-6). As explained below, deuterated R Tar-4 converts to deuterated R Tar-6. Except for deuterated R Tar-2 and deuterated R Tar-5, which has an acid to free base ratio of 1.6:1, and deuterated R Tar-3, which is a hemitartrate, the other deuterated R tartrate polymorphs are 1:1 salts.

Deuterated R Tar-1 is isolated from isopropanol. The XRPD diffractogram of deuterated R Tar-1 is substantially the same as Tar-1.

As indicated above, deuterated R Tar-2 and deuterated R Tar-5 are obtained from the reaction of deuterated R freebase with L-tartaric acid in ethyl acetate. Both deuterated R Tar-2 and deuterated R Tar-5 are crystalline salts. Deuterated R Tar-2 is the initial crystal that exists in the wet cake from filtration. Upon drying in a vacuum oven at room temperature, deuterated R Tar-2 transitions into deuterated R Tar-5. The stoichiometric ratio of base to acid of deuterated R Tar-2 and deuterated R Tar-5 is 1:1.6. The XRPD diffractograms of deuterated R Tar-2 and deuterated R Tar-5 is substantially the same as Tar-2 and Tar-5, respectively. The DSC-TGA of deuterated R Tar-5 is substantially the same as Tar-5. The DVS pattern of deuterated R Tar-5 is substantially the same as Tar-5.

Deuterated R Tar-3 is isolated from the reaction of deuterated R freebase and L-tartaric acid in THF. The deuterated R Tar-3 salt is crystalline. It is a hemitartrate, as the stoichiometric ratio of base: acid is 1.0:0.5. The XRPD diffractogram and DSC-TGA of deuterated R Tar-3 is substantially the same as the XRPD diffractogram and DSC-TGA of Tar-3, respectively. The DVS pattern of the deuterated R Tar-3 is substantially the same as that of Tar-3.

Deuterated R Tar-4 and deuterated R Tar-6 are obtained from the reaction of deuterated R freebase with L-tartaric acid in a mixture of acetone/water in a 9:1 ratio (v/v). Deuterated R Tar-4 is the crystal form in the wet cake from filtration and is converted into deuterated R Tar-6 upon drying in a vacuum oven at room temperature. Both deuterated R Tar-4 and deuterated R Tar-6 are crystalline. The stoichiometric ratio of deuterated R freebase to acid of deuterated R Tar-4 and deuterated R Tar-6 is 1:1. The XRPD diffractogram of deuterated R Tar-4 is substantially the same as the XRPD diffractogram of Tar-4. Further, the XRPD diffractogram and the DSC-TGA of deuterated R Tar-6 are substantially the same as the XRPD diffractogram and the DSC-TGA of Tar-6, respectively. The DVS pattern of the deuterated R Tar-6 salt is substantially the same as that of Tar-6.

2. (S)-2-(4-fluorophenyl)-2-(methylamino)cyclohexan-1-one tartrate and deuterated analogs thereof.

Substituting S freebase for freebase (R)-2-(4-fluorophenyl)-2-(methylamino)cyclohexan-1-one and D-tartaric acid for L-tartaric acid in the various solvents of isopropanol, ethyl acetate, tetrahydrofuran, and an acetone/water mixture of 9:1 (v/v), as described hereinabove, produces six crystalline polymorph salts. One product is obtained from isopropanol and will be identified as(S)-2-(4-fluorophenyl)-2-(methylamino)cyclohexan-1-one-D-Tar-1 (S tartrate-1 also designated as S Tar-1). Two products are isolated from ethyl acetate and will be identified(S)-2-(4-fluorophenyl)-2-(methylamino)cyclohexan-1-one-D-Tar-2 (S tartrate-2 also designated as S Tar-2) and(S)-2-(4-fluorophenyl)-2-(methylamino)cyclohexan-1-one-D-Tar-5 (S tartrate-5 also designated as S Tar-5). As discussed below, S Tar-2 converts to S Tar-5. A fourth product is isolated from tetrahydrofuran and will be identified as (R)-2-(4-fluorophenyl)-2-(methylamino)cyclohexan-1-one-D-Tar-3 (S tartrate-3 also designated as S Tar-3). Finally, two products are isolated from the acetone/water mixture and will be identified as (R)-2-(4-fluorophenyl)-2-(methylamino)cyclohexan-1-one-D-Tar-4 (S tartrate-4 also designated as S Tar-4) and(S)-2-(4-fluorophenyl)-2-(methylamino)cyclohexan-1-one-D-Tar-6 (S tartrate-6 also designated as S Tar-6). As explained below, S Tar-4 converts to S Tar-6. Except for S Tar-2 and S Tar-5, which has an acid to free base ratio of 1.6:1, and Tar-3 which is a hemitartrate, the other S tartrate polymorphs are 1:1 salts.

S Tar-1 is isolated from isopropanol and is crystalline. The XRPD diffractogram of S Tar-1 is substantially the same as Tar-1.

As indicated above, S Tar-2 and S Tar-5 are obtained from the reaction of deuterated R freebase with D-tartaric acid in ethyl acetate. Both S Tar-2 and S Tar-5 are crystalline salts. S Tar-2 is the initial crystal that exists in the wet cake from filtration. Upon drying in a vacuum oven at room temperature, S Tar-2 transitions into S Tar-5. The stoichiometric ratio of base to acid of S Tar-2 and S Tar-5 is 1: 1.6. The XRPD diffractograms of S Tar-2 and S Tar-5 is substantially the same as Tar-2 and Tar-5, respectively. The DSC-TGA of S Tar-5 is substantially the same as Tar-5. The DVS pattern of S Tar-5 is substantially the same as Tar-5.

S Tar-3 is isolated from the reaction of S freebase and D-tartaric acid in THF. The S Tar-3 salt is crystalline. It is a hemitartrate, as the stoichiometric ratio of base: acid is 1.0:0.5. The XRPD diffractogram and DSC-TGA of S Tar-3 is substantially the same as the XRPD diffractogram and DSC-TGA of Tar-3, respectively. The DVS pattern of the S Tar-3 is substantially the same as that of Tar-3.

S Tar-4 and S Tar-6 are obtained from the reaction of S freebase with D-tartaric acid in a mixture of acetone/water in a 9:1 ratio (v/v). S Tar-4 is the crystal form in the wet cake from filtration and is converted into S Tar-6 upon drying in a vacuum oven at room temperature. Both S Tar-4 and S Tar-6 are crystalline. The stoichiometric ratio of S freebase to acid of S Tar-4 and S Tar-6 is 1:1. The XRPD diffractogram of S Tar-4 is substantially the same as the XRPD diffractogram of Tar-4. Further, the XRPD diffractogram and the DSC-TGA of S Tar-6 are substantially the same as the XRPD diffractogram and the DSC-TGA of Tar-6, respectively. The DVS pattern of the S Tar-6 salt is substantially the same as that of Tar-6.

Substituting deuterated S freebase for freebase (R)-2-(4-fluorophenyl)-2-(methylamino)cyclohexan-1-one and D-tartaric acid for L-tartaric in the reaction described above in the various solvents: isopropanol, ethyl acetate, tetrahydrofuran, and an acetone/water mixture of 9:1 (v/v) produces six crystalline polymorph salts. One product is obtained from isopropanol and will be identified as deuterated(S)-2-(4-fluorophenyl)-2-(methylamino)cyclohexan-1-one-D-Tar-1 (deuterated S tartrate-1 also designated as deuterated S Tar-1). Two products are isolated from ethyl acetate and will be identified as deuterated(S)-2-(4-fluorophenyl)-2-(methylamino)cyclohexan-1-one-D-Tar-2 (deuterated S tartrate-2 also designated as deuterated S Tar-2) and deuterated(S)-2-(4-fluorophenyl)-2-(methylamino)cyclohexan-1-one-D-Tar-5 (deuterated S tartrate-5 also designated as deuterated S Tar-5). As discussed below, deuterated S Tar-2 converts to deuterated S Tar-5. A fourth product is isolated from tetrahydrofuran and will be identified as deuterated(S)-2-(4-fluorophenyl)-2-(methylamino)cyclohexan-1-one-D-Tar-3 (deuterated S tartrate-3 also designated as deuterated S Tar-3). Finally, two products are isolated from the acetone/water mixture and will be identified as deuterated(S)-2-(4-fluorophenyl)-2-(methylamino)cyclohexan-1-one-D-Tar-4 (deuterated S tartrate-4 also designated as deuterated S Tar-4) and deuterated(S)-2-(4-fluorophenyl)-2-(methylamino)cyclohexan-1-one-D-Tar-6 (deuterated S tartrate-6 also designated as deuterated S Tar-6). As explained below, deuterated S Tar-4 converts to deuterated S Tar-6. Except for deuterated S Tar-2 and deuterated S Tar-5, which has an acid to free base ratio of 1.6:1, and deuterated S Tar-3, which is a hemitartrate, the other deuterated S tartrate polymorphs are 1:1 salts.

Deuterated S Tar-1 is isolated from isopropanol and is crystalline. The XRPD diffractogram of deuterated S Tar-1 is substantially the same as Tar-1.

As indicated above, deuterated S Tar-2 and deuterated S Tar-5 are obtained from the reaction of deuterated S freebase with D-tartaric acid in ethyl acetate. Both deuterated S Tar-2 and deuterated S Tar-5 are crystalline salts. Deuterated S Tar-2 is the initial crystal that exists in the wet cake from filtration. Upon drying in a vacuum oven at room temperature, deuterated S Tar-2 transitions into deuterated S Tar-5. The stoichiometric ratio of base to acid of deuterated S Tar-2 and deuterated S Tar-5 is 1: 1.6. The XRPD diffractograms of deuterated S Tar-2 and deuterated S Tar-5 is substantially the same as Tar-2 and Tar-5, respectively. The DSC-TGA of deuterated S Tar-5 is substantially the same as Tar-5. The DVS pattern of deuterated S Tar-5 is substantially the same as that of Tar-5.

Deuterated S Tar-3 is isolated from the reaction of deuterated S freebase and D-tartaric acid in THF. The deuterated S Tar-3 salt is crystalline. It is a hemitartrate, as the stoichiometric ratio of base: acid is 1.0:0.5. The XRPD diffractogram and DSC-TGA of deuterated S Tar-3 is substantially the same as the XRPD diffractogram and DSC-TGA of Tar-3, respectively. The DVS pattern of the deuterated S Tar-3 is substantially the same as that of Tar-3.

Deuterated S Tar-4 and deuterated S Tar-6 are obtained from the reaction of deuterated S freebase with D-tartaric acid in a mixture of acetone/water in a 9:1 ratio (v/v). Deuterated S Tar-4 is the crystal form in the wet cake from filtration and is converted into deuterated S Tar-6 upon drying in a vacuum oven at room temperature. Both deuterated S Tar-4 and deuterated S Tar-6 are crystalline. The stoichiometric ratio of deuterated S freebase to acid with respect to deuterated S Tar-4 and deuterated S Tar-6 is 1:1. The XRPD diffractogram of deuterated S Tar-4 is substantially the same as the XRPD diffractogram of Tar-4. Further, the XRPD diffractogram and the DSC-TGA of deuterated S Tar-6 are substantially the same as the XRPD diffractogram and the DSC-TGA of Tar-6, respectively. The DVS pattern of the deuterated S Tar-6 salt is substantially the same as that of Tar-6.

(h) 1. (R)-2-(4-fluorophenyl)-2-(methylamino)cyclohexan-1-one fumarate and its deuterated analogs

Isopropanol, acetonitrile, ethyl acetate, tetrahydrofuran, and an acetone/water mixture of 9:1 v/v (0.25 mL of each solvent) were added to five different glass vials each containing about 40 mg of freebase (R)-2-(4-fluorophenyl)-2-(methylamino)cyclohexan-1-one. One molar equivalent of fumaric acid was added to each vial and the contents were stirred at room temperature. A solid product was collected from each and vacuum dried at room temperature. As determined by XRPD, the products isolated from each of the vials were crystalline and shared the same XRPD diffractogram. This product will be identified herein as (R)-2-(4-fluorophenyl)-2-(methylamino)cyclohexan-1-one Fum-1 (fumarate-1, also denoted as Fum-1). Using proton NMR, it was determined that the stoichiometry of base to acid is 1:1 in Fum-1.

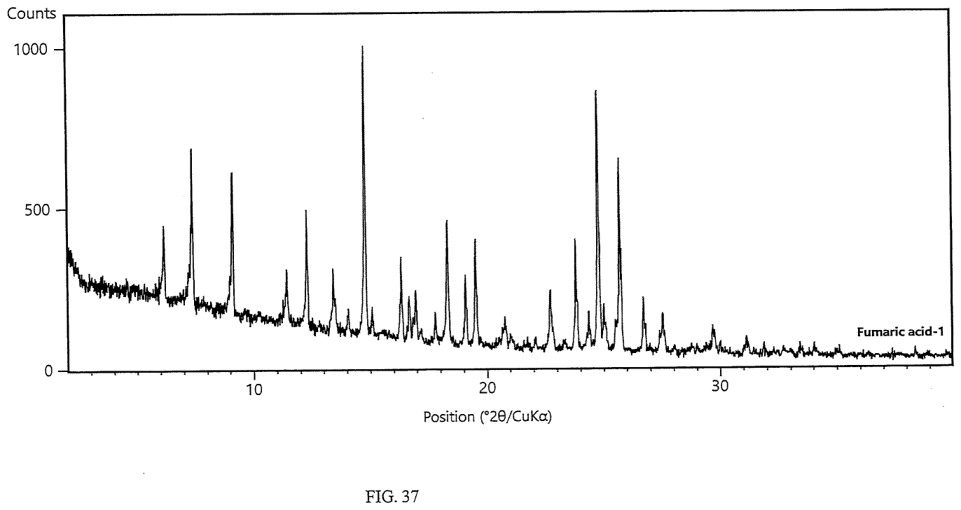

The Fum-1 obtained was analyzed by XRPD. The XRPD diffractogram of Fum-1 is depicted in FIG. 37. The peak values and intensity of the XPRD of the Fum-1 salt are provided below in Table 24.

TABLE 24
XRPD peak values for FIG. 37 (Fum-1).

| Peak # | Pos. [°2θ] | Intensity |
|---|---|---|
| 1 | 6.1581 | 236.76 |
| 2 | 7.3847 | 509.95 |
| 3 | 9.1154 | 452.82 |
| 4 | 11.4195 | 168.82 |
| 5 | 12.2962 | 332.21 |
| 6 | 13.4078 | 191.18 |
| 7 | 14.0368 | 56.65 |
| 8 | 14.7822 | 918.33 |
| 9 | 15.0745 | 51.31 |
| 10 | 16.3204 | 252.88 |
| 11 | 16.6445 | 123.03 |
| 12 | 16.9340 | 144.03 |
| 13 | 17.7696 | 75.43 |
| 14 | 18.2997 | 384.61 |
| 15 | 19.0686 | 216.92 |
| 16 | 19.5086 | 340.04 |
| 17 | 20.7804 | 77.90 |
| 18 | 21.0042 | 28.07 |
| 19 | 22.6948 | 164.50 |
| 20 | 23.8068 | 346.80 |
| 21 | 24.3575 | 120.94 |
| 22 | 24.7711 | 822.17 |
| 23 | 25.0041 | 143.79 |
| 24 | 25.5451 | 72.80 |
| 25 | 25.6922 | 608.20 |
| 26 | 26.6969 | 148.46 |
| 27 | 27.4101 | 35.26 |
| 28 | 27.5265 | 122.68 |
| 29 | 29.6765 | 80.57 |
| 30 | 31.1132 | 37.36 |
| 31 | 31.8859 | 14.76 |
| 32 | 33.9389 | -17.92 |
| 33 | 34.0255 | 20.23 |
| 34 | 35.0150 | 12.67 |
| 35 | 38.3871 | 13.83 |

As shown, in an embodiment, this Fum-1 salt can be characterized by peaks in the X-Ray diffractogram at 14.8, 24.8, and 25.7±0.5 ° 2θ. In another embodiment, this salt can be characterized by peaks at 7.4, 9.1, 14.8, 24.8, and 25.7±0.5 ° 2θ. In another embodiment, this salt can be characterized by peaks at 7.4, 9.1, 12.3, 14.8, 16.3, 18.3, 19.5, 23.8, 24.8, and 25.7±0.5 ° 2θ. In another embodiment, this salt can be characterized by peaks at 6.2, 7.4, 9.1, 11.4, 12.3, 13.4, 14.0, 14.8, 15.1, 16.3, 16.7, 16.9, 17.8, 18.3, 19.1, 19.5, 20.8, 21.0, 22.7, 23.8, 24.4, 24.8, 25.0, 25.5, 25.7, 27.0, 27.4, 27.5, 29.7, 31.1, 31.9, 33.9, 34.0, 35.0, and 38.3±0.5 ° 2θ.

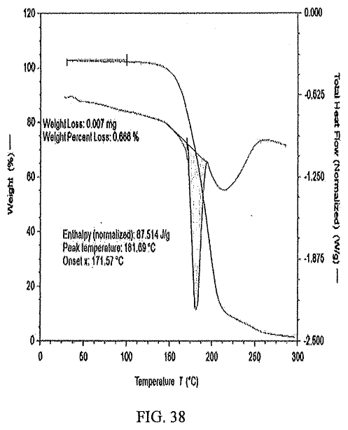

The Fum-1 salt was analyzed by DSC-TGA using the methodology described hereinabove. The data is depicted in FIG. 38. There was a weight loss of about 0.66% by 100 °C. In addition, the DSC thermogram of Fum-1 exhibited an intense peak temperature at about 181.7 °C.

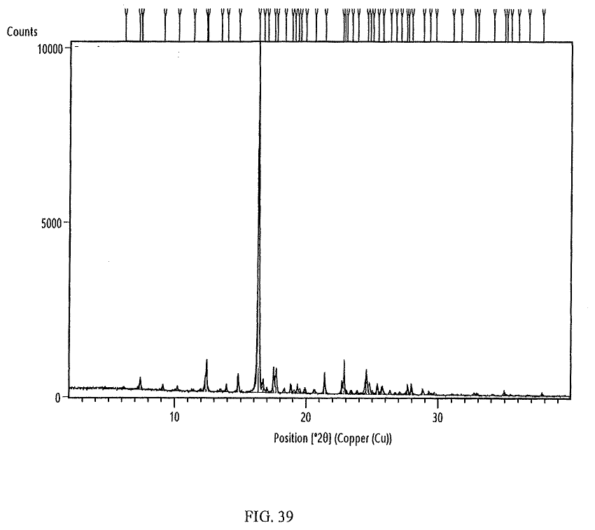

A different polymorph designated herein as (R)-2-(4-fluorophenyl)-2-(methylamino)cyclohexan-1-one Fum-2 (fumarate-2, also denoted as Fum-2) was prepared by reacting the free base directly with fumaric acid. Specifically, an equivalent of freebase was placed into a vial to which was added about 1.0 molar equivalent of fumaric acid. After initial mixing, about 10 volumes of ethyl acetate was added at room temperature to further aid in the mixing. After the reaction was completed, the product was collected by filtration, dried under vacuum at room temperature, and analyzed by XRPD. The XRPD diffractogram obtained was different from that of Fum-1 and this new form was designated as Fum-2. The XRPD diffractogram of Fum-2 is depicted in FIG. 39. The peak values and intensity of the XPRD of the Fum-2 salt are provided below in Table 25.

TABLE 25
XRPD peak values for FIG. 39 (Fum-2).

| Peak # | Pos. [°2θ] | Intensity |
|---|---|---|
| 1 | 6.1540 | 54.58 |
| 2 | 7.2296 | 50.16 |
| 3 | 7.3866 | 294.12 |
| 4 | 7.4479 | 71.07 |
| 5 | 9.1206 | 152.11 |
| 6 | 10.1946 | 127.50 |
| 7 | 11.3762 | 43.85 |
| 8 | 12.3290 | 330.04 |
| 9 | 12.4154 | 840.29 |
| 10 | 13.4500 | 65.15 |
| 11 | 13.9203 | 188.62 |
| 12 | 14.7970 | 534.59 |
| 13 | 16.3114 | 10165.32 |
| 14 | 16.6661 | 298.61 |
| 15 | 16.9675 | 126.38 |
| 16 | 17.4916 | 681.67 |
| 17 | 17.7000 | 706.15 |
| 18 | 18.3217 | 123.64 |
| 19 | 18.8542 | 239.91 |
| 20 | 19.0963 | 57.62 |
| 21 | 19.3479 | 256.85 |
| 22 | 19.5173 | 114.57 |
| 23 | 19.9167 | 148.85 |
| 24 | 20.6202 | 109.77 |
| 25 | 21.3875 | 589.96 |
| 26 | 22.6954 | 369.88 |
| 27 | 22.8453 | 980.12 |
| 28 | 23.0019 | 88.85 |
| 29 | 23.3932 | 106.16 |
| 30 | 23.8299 | 108.69 |
| 31 | 24.5593 | 688.44 |
| 32 | 24.7886 | 332.62 |
| 33 | 24.9816 | 76.90 |
| 34 | 25.3715 | 298.80 |
| 35 | 25.7410 | 228.95 |
| 36 | 26.3219 | 120.96 |
| 37 | 26.7332 | 56.87 |
| 38 | 27.1135 | 79.46 |
| 39 | 27.5581 | 67.00 |
| 40 | 27.6782 | 296.12 |
| 41 | 27.9552 | 323.20 |
| 42 | 28.8117 | 159.66 |
| 43 | 29.2920 | 103.76 |
| 44 | 29.7591 | 43.56 |
| 45 | 31.0436 | 34.47 |
| 46 | 31.6500 | 20.70 |
| 47 | 32.6959 | 51.23 |
| 48 | 32.9196 | 61.90 |
| 49 | 34.1011 | 33.39 |
| 50 | 34.9384 | 148.14 |
| 51 | 35.0892 | 21.33 |
| 52 | 35.4102 | 21.77 |
| 53 | 35.9672 | 24.75 |
| 54 | 36.7310 | 30.00 |
| 55 | 37.7763 | 90.16 |

As shown, in an embodiment, this Fum-2 salt can be characterized by peaks in the X-Ray diffractogram at 12.4, 16.3, and 22.8±0.5 ° 2θ. In another embodiment, this salt can be characterized by peaks at 12.4, 16.3, 17.7, 22.8, and 24.6±0.5 ° 2θ. In another embodiment, this salt can be characterized by peaks at 12.4, 14.8, 16.3, 17.5, 17.7, 21.4, 22.7, 22.8, 24.6, and 24.8±0.5 ° 2θ. In another embodiment, this salt can be characterized by peaks at 6.2, 7.3, 7.4 (7.39), 7.4 (7.45), 9.1, 10.1, 11.3, 12.3, 12.4, 13.5, 13.9.0, 14.8, 16.3, 16.7, 17.0, 17.5, 17.7, 18.3, 18.9, 19.1, 19.3, 19.5, 19.9, 20.6, 21.4, 22.7, 22.8, 23.0, 23.4, 23.8, 24.6, 24.8, 25.0, 25.4, 25.7, 26.3, 26.7, 27.1, 27.6, 27.7, 28.0, 28.8, 29.3, 29.8, 31.0, 31.7, 32.7, 32.9, 33.9, 34.1, 34.9, 35.1, 35.4, 36.0, 36.7, and 37.8±0.5 ° 2θ.

The Fum-2 thus obtained was determined to have a 1:0.65 stoichiometric ratio of base: acid by proton NMR.

A better sample of Fum-2 was obtained by reacting a molar equivalent of freebase with 0.5 molar equivalent of fumaric acid in 5 volumes of ethyl acetate. The contents of the vial were stirred and the resulting solid was collected. A proton NMR spectrum was recorded to confirm that the stoichiometric ratio of base to acid was 2:1. The XPRD of this sample essentially matched the XRPD of Fum-2 depicted in FIG. 39.

There are at least two crystalline polymorph salts of deuterated (R)-2-(4-fluorophenyl)-2-(methylamino)cyclohexan-1-one fumarates. Substituting deuterated R freebase for freebase (R)-2-(4-fluorophenyl)-2-(methylamino)cyclohexan-1-one in the reaction with fumaric acid in isopropanol, acetonitrile, ethyl acetate, tetrahydrofuran, and an acetone/water mixture of 9: 1 (v/v), as described above, produces one polymorph, designated as deuterated (R)-2-(4-fluorophenyl)-2-(methylamino)cyclohexan-1-one fumarate-1 (deuterated R fumarate-1 or also identified as deuterated R Fum-1). The stoichiometry of deuterated R freebase to acid is 1:1. The XRPD diffractogram and DSC-TGA of deuterated Fum-1 are substantially the same as the XRPD diffractogram and DSC-TGA of Fum-1, respectively. In addition, the DVS patterns of deuterated R Fum-1 is substantially the same as that of Fum-1.

A second polymorph is obtained by substituting deuterated R freebase for freebase (R)-2-(4-fluorophenyl)-2-(methylamino)cyclohexan-1-one in the reaction with fumaric acid, as described above to form deuterated (R)-2-(4-fluorophenyl)-2-(methylamino)cyclohexan-1-one fumarate-2 (deuterated R fumarate-2 or also identified as deuterated R Fum-2). The XRPD diffractogram of the deuterated R Fum-2 is substantially the same as the XRPD diffractogram of Fum-2. The deuterated R Fum-2 has a 2:1 stoichiometric ratio of base: acid.

2. (S)-2-(4-fluorophenyl)-2-(methylamino)cyclohexan-1-one fumarate and its deuterated analogs

There are at least two crystalline polymorph salts of each of(S)-2-(4-fluorophenyl)-2-(methylamino)cyclohexan-1-one fumarates and deuterated(S)-2-(4-fluorophenyl)-2-(methylamino)cyclohexan-1-one fumarates. Substituting S freebase and deuterated S freebase for freebase (R)-2-(4-fluorophenyl)-2-(methylamino)cyclohexan-1-one in the reaction with fumaric acid in isopropanol, acetonitrile, ethyl acetate, tetrahydrofuran, and an acetone/water mixture of 9: 1 (v/v), as described above, produces the first polymorph, (S)-2-(4-fluorophenyl)-2-(methylamino)cyclohexan-1-one fumarate-1 (S fumarate-1, also identified as S Fum-1) and deuterated(S)-2-(4-fluorophenyl)-2-(methylamino)cyclohexan-1-one fumarate-1 (deuterated S fumarate-1, also identified as deuterated S Fum-1), respectively. The stoichiometry of deuterated S freebase to acid with respect to the reaction forming S Fum-1 and deuterated S Fum-1 is 1:1. The XRPD diffractogram and DSC-TGA of both S Fum-1 and deuterated S Fum-1 are substantially the same as the XRPD diffractogram and DSC-TGA of Fum-1, respectively. In addition, the DVS patterns of S Fum-1 and deuterated S Fum-1 are substantially the same as that of Fum-1.

A second polymorph is obtained by substituting S freebase and deuterated S freebase for freebase (R)-2-(4-fluorophenyl)-2-(methylamino)cyclohexan-1-one in the reaction with fumaric acid, as described above, to form(S)-2-(4-fluorophenyl)-2-(methylamino)cyclohexan-1-one fumarate-2 (S fumarate-2, also identified as S Fum-2) and deuterated(S)-2-(4-fluorophenyl)-2-(methylamino)cyclohexan-1-one fumarate-2 (deuterated S fumarate-2, also identified as deuterated S Fum-2) The XRPD diffractogram of both S Fum-2 and deuterated S Fum-2 are substantially the same as the XRPD diffractogram of Fum-2. Both S Fum-2 and deuterated S Fum-2 have a 2:1 stoichiometric ratio of base: acid.

(i) 1. (R)-2-(4-fluorophenyl)-2-(methylamino)cyclohexan-1-one citrate and its deuterated analogs

About 0.25 mL of ethyl acetate was added to a glass vial containing about 40 mg of freebase (R)-2-(4-fluorophenyl)-2-(methylamino)cyclohexan-1-one. One molar equivalent of citric acid was added to the vial, and the contents were stirred at room temperature. A solid product was observed and was collected and vacuum dried at room temperature. The initially collected wet crystals exhibited one XRPD pattern identified as (R)-2-(4-fluorophenyl)-2-(methylamino)cyclohexan-1-one Cit-1 (citrate-1, also designated as Cit-1), but this pattern transitioned to a different XRPD pattern identified as (R)-2-(4-fluorophenyl)-2-(methylamino)cyclohexan-1-one Cit-2 (citrate-2, also designated as Cit-2) upon drying in a vacuum oven at room temperature. As determined by XRPD, both products were crystalline.

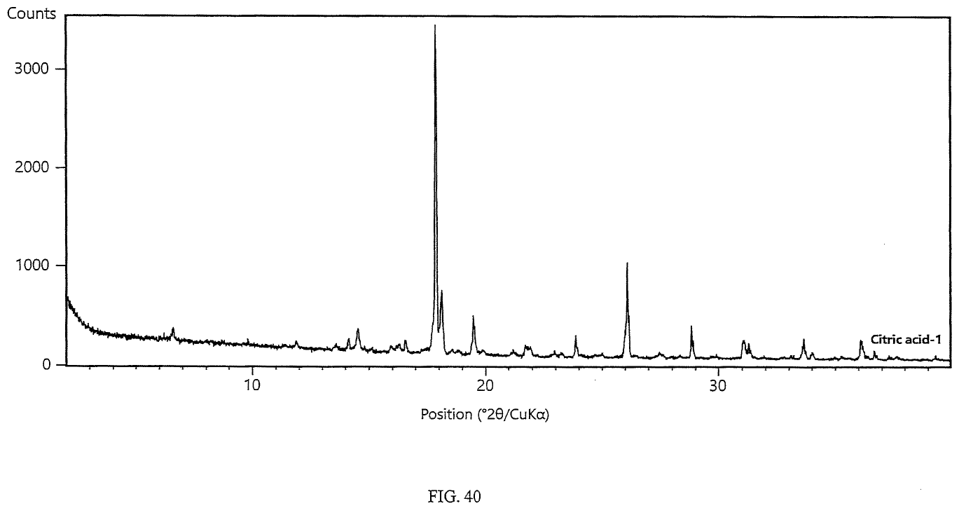

The XRPD diffractogram of Cit-1 is depicted in FIG. 40. The peak values and intensity of the XPRD of the Cit-1 salt are provided below in Table 26.

TABLE 26
XRPD peak values for FIG. 40 (Cit-1).

| Peak # | Pos. [°2θ] | Intensity |
|---|---|---|
| 1 | 2.0371 | 334.78 |
| 2 | 6.5825 | 120.96 |
| 3 | 11.8395 | 25.15 |
| 4 | 13.5643 | 39.71 |
| 5 | 14.1277 | 108.06 |
| 6 | 14.5230 | 216.73 |
| 7 | 15.9498 | 46.94 |
| 8 | 16.1700 | 43.40 |
| 9 | 16.2782 | 59.24 |
| 10 | 16.5632 | 101.30 |
| 11 | 17.8589 | 3260.50 |
| 12 | 18.1133 | 621.45 |
| 13 | 19.4696 | 371.40 |
| 14 | 19.8746 | 47.40 |
| 15 | 21.1769 | 38.35 |
| 16 | 21.7212 | 88.04 |
| 17 | 21.8800 | 66.57 |
| 18 | 22.9451 | 37.06 |
| 19 | 23.2493 | 33.26 |
| 20 | 23.8653 | 218.05 |
| 21 | 24.8723 | 18.03 |
| 22 | 26.0797 | 935.09 |
| 23 | 27.4941 | 41.76 |
| 24 | 28.8328 | 321.39 |
| 25 | 28.9021 | 171.17 |
| 26 | 29.8506 | 17.27 |
| 27 | 31.0629 | 173.63 |
| 28 | 31.2851 | 151.20 |
| 29 | 33.6284 | 136.11 |
| 30 | 34.0054 | 64.22 |
| 31 | 36.1035 | 177.65 |
| 32 | 36.3313 | 33.30 |
| 33 | 36.6836 | 86.84 |
| 34 | 37.3301 | 23.25 |
| 35 | 37.6551 | 29.47 |
| 36 | 39.3146 | 26.01 |

As shown, in an embodiment, this Cit-1 salt can be characterized by peaks in the X-Ray diffractogram at 17.9, 18.1, and 26.1±0.5 ° 2θ. In another embodiment, this salt can be characterized by peaks at 2.0, 17.9, 18.1, 19.5, and 26.1±0.5 ° 2θ. In another embodiment, this salt can be characterized by peaks at 2.0, 17.9, 18.1, 19.5, 23.9, 26.1, 28.8, 28.9, 31.1, and 36.1±0.5 ° 2θ. In another embodiment, this salt can be characterized by peaks at 2.0, 6.6, 11.8, 13.6, 14.1, 14.5, 15.9, 16.2, 16.3, 16.6, 17.9, 18.1, 19.5, 16.7, 17.0, 17.5, 17.8, 18.3, 18.9, 19.1, 19.3, 19.5, 19.9, 21.2, 21.7, 21.9, 22.9, 23.2, 23.9, 24.9, 26.1, 27.5, 28.8, 28.9, 29.9, 31.1, 31.3, 33.6, 34.0, 36.1, 36.3, 36.7, 37.3, 37.7, and 39.3±0.5 ° 2θ.

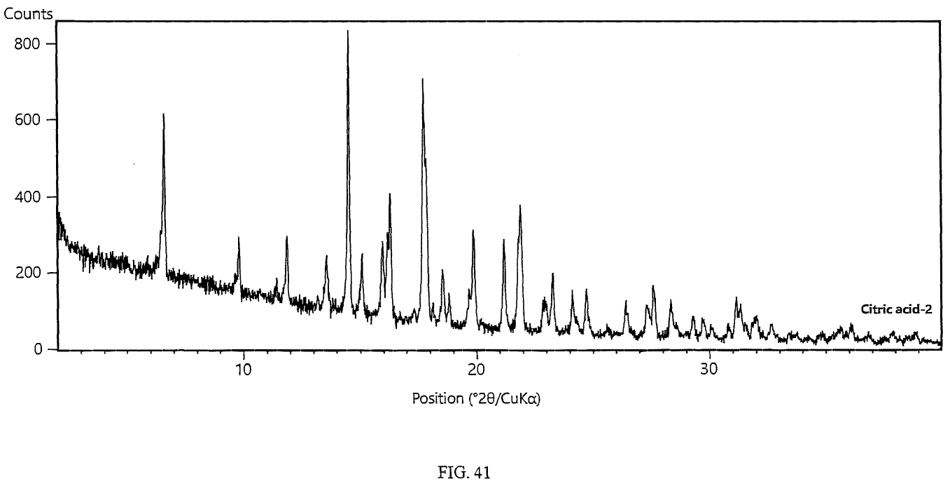

As determined by proton NMR, the stoichiometric ratio of base: acid was found to be 1:1 for Cit-2 and by inference, 1:1 for Cit-1. The XRPD diffractogram of Cit-2 is depicted in FIG. 41. The peak values and intensity of the XPRD of the Cit-2 salt are provided below in Table 27.

TABLE 27
XRPD peak values for FIG. 41 (Cit-2).

| Peak # | Pos. [°2θ] | Intensity |
|---|---|---|
| 1 | 6.5998 | 425.57 |
| 2 | 9.6551 | 24.05 |
| 3 | 9.7957 | 148.50 |
| 4 | 11.4014 | 37.20 |
| 5 | 11.8503 | 169.03 |
| 6 | 13.5554 | 125.76 |
| 7 | 14.5195 | 760.83 |
| 8 | 15.0763 | 139.04 |
| 9 | 15.9444 | 179.18 |
| 10 | 16.2729 | 297.32 |
| 11 | 17.3045 | 22.44 |
| 12 | 17.7152 | 606.91 |
| 13 | 17.8538 | 381.10 |
| 14 | 18.1082 | 27.59 |
| 15 | 18.5099 | 125.44 |
| 16 | 18.8195 | 66.29 |
| 17 | 19.6964 | 64.12 |
| 18 | 19.8697 | 242.69 |
| 19 | 21.1815 | 223.83 |
| 20 | 21.8268 | 157.19 |
| 21 | 21.9332 | 239.06 |
| 22 | 22.9002 | 69.82 |
| 23 | 23.2813 | 130.83 |
| 24 | 24.1037 | 99.68 |
| 25 | 24.2538 | 28.89 |
| 26 | 24.6810 | 87.84 |
| 27 | 26.3792 | 76.31 |
| 28 | 27.2855 | 76.35 |
| 29 | 27.5593 | 130.34 |
| 30 | 28.3165 | 85.41 |
| 31 | 28.5502 | 21.61 |
| 32 | 29.2935 | 48.34 |
| 33 | 29.7160 | 42.80 |
| 34 | 30.0847 | 25.69 |
| 35 | 30.7944 | 28.98 |
| 36 | 31.1373 | 109.33 |
| 37 | 31.2900 | 61.91 |
| 38 | 31.4909 | 25.03 |
| 39 | 31.9655 | 48.91 |
| 40 | 32.6336 | 36.25 |
| 41 | 35.5599 | 22.68 |
| 42 | 36.1405 | 27.24 |
| 43 | 36.8110 | 13.28 |
| 44 | 37.8493 | 22.06 |
| 45 | 38.8677 | 23.78 |

As shown, in an embodiment, this Cit-2 salt can be characterized by peaks in the X-Ray diffractogram at 6.6, 14.5, and 17.7±0.5 ° 2θ. In another embodiment, this salt can be characterized by peaks at 6.6, 14.5, 16.3, 17.7, and 17.9±0.5 ° 2θ. In another embodiment, this salt can be characterized by peaks at 6.6, 11.9, 14.5, 15.9, 16.3, 17.7, 17.9, 19.9, 21.2, and 21.9±0.5 ° 2θ. In another embodiment, this salt can be characterized by peaks at 6.6, 9.7, 9.8, 11.4, 11.9, 13.6, 14.5, 15.1, 15.9, 16.3, 17.3, 17.7, 17.9, 18.1, 18.5, 18.8, 19.7, 19.9, 21.2, 21.8, 21.9, 22.9, 23.3, 23.9, 24.1, 24.3, 24.7, 26.4, 27.3, 27.6, 28.3, 28.6, 29.3, 29.7, 30.1, 30.8, 31.1, 31.3, 31.5, 32.0, 32.6, 35.6, 36.1, 36.8, 37.8, and 38.9±0.5 ° 2θ.

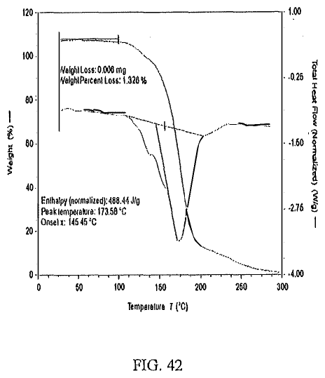

The Cit-2 salt was analyzed by DSC-TGA using the methodology described hereinabove. The data is depicted in FIG. 42. There was a weight loss of about 1.33% when the temperature was raised from room temperature to 100 °C. In addition, the DSC thermogram of the Cit-2 salt exhibited a very broad peak temperature at about 173.6 °C.

Substituting deuterated R freebase for freebase (R)-2-(4-fluorophenyl)-2-(methylamino)cyclohexan-1-one in the reaction with citric acid described above produces a crystalline polymorph salt of deuterated (R)-2-(4-fluorophenyl)-2-(methylamino)cyclohexan-1-one citrate-1 (deuterated R citrate-1, also designated as deuterated R-Cit-1). The XRPD diffractogram of the initially collected wet crystals of deuterated R-Cit-1 is substantially the same as the XRPD diffractogram of Cit-1.

As with Cit-1, the deuterated R-Cit-1 transforms into a different crystalline polymorph salt upon drying in a vacuum oven at room temperature. The transformed deuterated R-Cit-1 is designated as deuterated (R)-2-(4-fluorophenyl)-2-(methylamino)cyclohexan-1-one citrate-2 (deuterated R citrate-2, also identified as deuterated R-Cit-2). The XRPD and the DSC-TGA of deuterated R-Cit-2 are substantially the same as the XRPD and the DSC-TGA of Cit-2, respectively. The DVS pattern of the deuterated R-Cit-2 salt is substantially the same as that of Cit-2.

The stoichiometric ratio of base: acid for both deuterated R-Cit-1 and deuterated R Cit-2 is 1:1.

2. (S)-2-(4-fluorophenyl)-2-(methylamino)cyclohexan-1-one citrate and its deuterated analogs

Substituting S freebase and deuterated S freebase for freebase (R)-2-(4-fluorophenyl)-2-(methylamino)cyclohexan-1-one in the reaction with citric acid described above produces a crystalline polymorph salt of(S)-2-(4-fluorophenyl)-2-(methylamino)cyclohexan-1-one citrate-1 (S citrate-1, also designated as S Cit-1) and deuterated(S)-2-(4-fluorophenyl)-2-(methylamino)cyclohexan-1-one citrate-1 (deuterated S citrate-1, also designated as deuterated S Cit-1). The XRPD diffractogram of the initially collected wet crystals of both S Cit-1 and deuterated S-Cit-1 is substantially the same as the XRPD diffractogram of Cit-1.

As with Cit-1, both S Cit-1 and deuterated S Cit-1 transform into a different crystalline polymorph salts upon drying in a vacuum oven at room temperature. The transformed S Cit-1 and deuterated S Cit-1 are designated as(S)-2-(4-fluorophenyl)-2-(methylamino)cyclohexan-1-one citrate-2 (S citrate-2, also identified as S Cit-2) and deuterated(S)-2-(4-fluorophenyl)-2-(methylamino)cyclohexan-1-one citrate-2 (deuterated S citrate-2, also identified as deuterated S Cit-2), respectively. The XRPD and the DSC-TGA of both S-Cit-2 and deuterated R-Cit-2 are substantially the same as the XRPD and the DSC-TGA of Cit-2, respectively. The DVS patterns of both S Cit-2 and deuterated S-Cit-2 salt are substantially the same as that of Cit-2.

The stoichiometric ratio of base: acid for both S Cit-1 and deuterated S Cit-1 as well as S Cit-2 and deuterated S Cit-2 is 1:1.

(j) 1. (R)-2-(4-fluorophenyl)-2-(methylamino)cyclohexan-1-one glycolate and deuterated analog

Isopropanol, acetonitrile, ethyl acetate, and tetrahydrofuran (0.25 mL of each solvent) were added to four different glass vials each containing about 40 mg of freebase (R)-2-(4-fluorophenyl)-2-(methylamino)cyclohexan-1-one. One molar equivalent of glycolic acid was added to each vial and the contents were stirred at room temperature. A solid product was observed in each and was collected from each and vacuum dried at room temperature. XRPD analysis revealed that the products isolated from each of the vials were crystalline and that all the samples exhibited the same XRPD diffraction pattern. This product will be identified herein as (R)-2-(4-fluorophenyl)-2-(methylamino)cyclohexan-1-one Glyc-1 (glycolate-1, also denoted as Glyc-1). As determined by proton NMR, the stoichiometric ratio of base to acid was 1:1.

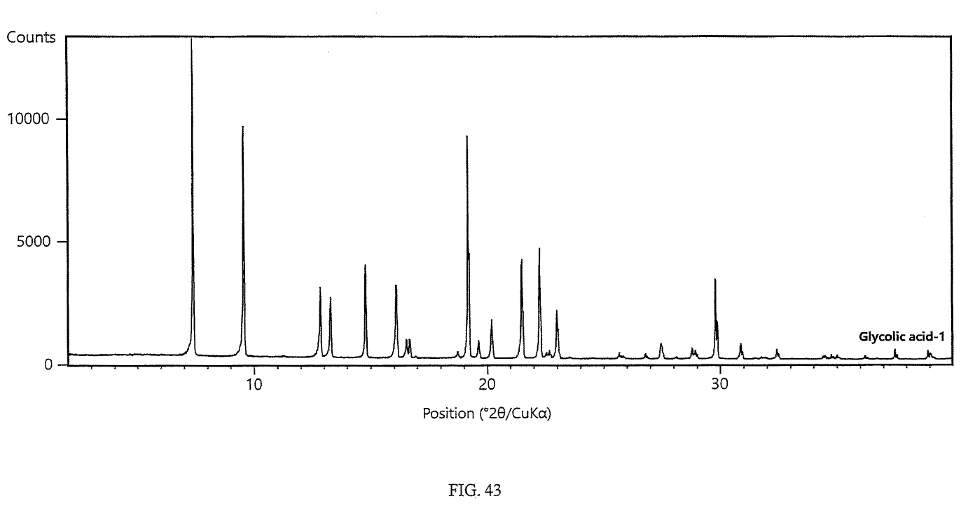

The Glyc-1 obtained was analyzed by XRPD. The XRPD diffractogram of Glyc-1 is depicted in FIG. 43. The peak values and intensity of the XPRD of the Glyc-1 salt are provided below in Table 28.

TABLE 28
XRPD peak values for FIG. 43 (Glyc-1).

| Peak # | Pos. [°2θ] | Intensity |
|---|---|---|
| 1 | 7.3781 | 9286.28 |
| 2 | 9.5595 | 10460.89 |
| 3 | 12.8368 | 1735.88 |
| 4 | 13.2781 | 1880.11 |
| 5 | 14.4115 | -626.08 |
| 6 | 14.7740 | 4919.75 |
| 7 | 16.0860 | 2362.49 |
| 8 | 16.5106 | 848.43 |
| 9 | 16.6600 | 925.80 |
| 10 | 18.7071 | 182.12 |
| 11 | 19.1661 | 13246.06 |
| 12 | 19.6151 | 375.83 |
| 13 | 20.1742 | 1140.73 |
| 14 | 21.4695 | 2499.35 |
| 15 | 22.2322 | 6670.06 |
| 16 | 22.3004 | 2762.17 |
| 17 | 22.5229 | 254.88 |
| 18 | 22.6719 | 186.66 |
| 19 | 22.9726 | 1369.79 |
| 20 | 23.0401 | 1013.30 |
| 21 | 23.5351 | 40.17 |
| 22 | 25.6547 | 190.90 |
| 23 | 25.8124 | 63.39 |
| 24 | 26.7756 | 109.60 |
| 25 | 27.4726 | 544.78 |
| 26 | 28.0609 | 55.59 |
| 27 | 28.7794 | 291.81 |
| 28 | 28.9281 | 600.97 |
| 29 | 29.7951 | 5585.26 |
| 30 | 29.8768 | 2482.86 |
| 31 | 30.8563 | 416.03 |
| 32 | 31.7534 | 69.51 |
| 33 | 31.9014 | 64.41 |
| 34 | 32.4036 | 252.58 |
| 35 | 32.4965 | 107.11 |
| 36 | 33.7047 | 19.35 |
| 37 | 34.4871 | 132.46 |
| 38 | 34.7451 | 139.98 |
| 39 | 34.9954 | 131.40 |
| 40 | 36.2036 | 174.13 |
| 41 | 36.3003 | 72.59 |
| 42 | 37.4923 | 737.45 |
| 43 | 37.5894 | 298.89 |
| 44 | 38.9064 | 634.04 |
| 45 | 39.0121 | 299.56 |

As shown, in an embodiment, this Glyc-1 salt can be characterized by intense peaks in the X-Ray diffractogram at 7.4, 9.6, and 19.2±0.5 ° 2θ. In another embodiment, this salt can be characterized by peaks at 7.4, 9.6, 19.2, 22.2, and 29.8±0.5 ° 2θ. In another embodiment, this salt can be characterized by peaks at 7.4, 9.6, 14.8, 16.1, 19.2, 21.5, 22.2, 22.3, and 29.8, 29.9±0.5 ° 2θ. In another embodiment, this salt can be characterized by peaks at 7.4, 9.6, 12.8, 13.3, 14.4, 14.8, 16.1, 16.5, 16.7, 18.7, 19.2, 19.6, 20.2, 21.5, 22.2, 22.3, 22.5, 22.7, 23.0 (22.97), 23.0 (23.04), 23.5, 25.7, 25.8, 26.8, 27.5, 28.1, 28.8, 28.9, 29.8, 29.9, 30.9, 31.8, 31.9, 32.4, 32.5, 33.7, 34.5, 34.7, 35.0, 36.2, 36.3, 37.5, 37.6, 38.9, and 39.0±0.5 ° 2θ.

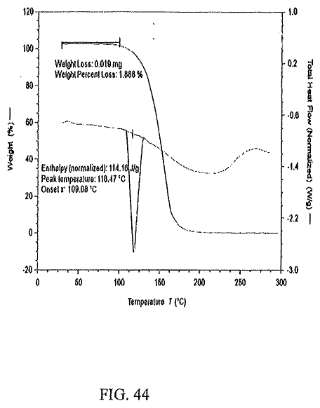

The Glyc-1 salt was analyzed by DSC-TGA using the methodology described hereinabove. The data is depicted in FIG. 44. There was a weight loss of about 1.89% when the temperature was raised from room temperature to 100 °C. In addition, the DSC thermogram of the Glyc-1 salt exhibited an intense peak temperature at about 118.5 °C.

Substituting deuterated R free base for (R)-2-(4-fluorophenyl)-2-(methylamino)cyclohexan-1-one in the reaction with glycolic acid, as described hereinabove produces deuterated (R)-2-(4-fluorophenyl)-2-(methylamino)cyclohexan-1-one glycolate-1 (deuterated R glycolate-1, also identified as deuterated R Glyc-1). The XRPD diffractogram and DSC-TGA of deuterated R Glyc-1 are substantially the same as the XRPD diffractogram and DSC-TGA of Glyc-1, respectively. The DVS pattern of the deuterated R Glyc-1 salt is substantially the same as that of Glyc-1.

2. (S)-2-(4-fluorophenyl)-2-(methylamino)cyclohexan-1-one glycolate and deuterated analog

Substituting S free base and deuterated S free base for (R)-2-(4-fluorophenyl)-2-(methylamino)cyclohexan-1-one in the reaction with glycolic acid, as described hereinabove, produces(S)-2-(4-fluorophenyl)-2-(methylamino)cyclohexan-1-one glycolate-1 (S glycolate-1, also identified as S Glyc-1) and deuterated(S)-2-(4-fluorophenyl)-2-(methylamino)cyclohexan-1-one glycolate-1 (deuterated S glycolate-1, also identified as deuterated S Glyc-1). The XRPD diffractogram and DSC-TGA of both S Glyc-1 and deuterated S Glyc-1 are substantially the same as the XRPD diffractogram and DSC-TGA of Glyc-1, respectively. The DVS patterns of both S Glyc-1 and deuterated S Glyc-1 salt are substantially the same as that of Glyc-1.

(k) 1. (R)-2-(4-fluorophenyl)-2-(methylamino)cyclohexan-1-one-L-malate and its deuterated analog

Ethyl acetate (0.25 mL) was added to a glass vial containing about 40 mg of freebase (R)-2-(4-fluorophenyl)-2-(methylamino)cyclohexan-1-one. One molar equivalent of L-malic acid was added to the vial and the contents were stirred at room temperature. A solid product was observed and was collected and vacuum dried at room temperature. The product isolated from the vial was crystalline and will be identified herein as (R)-2-(4-fluorophenyl)-2-(methylamino)cyclohexan-1-one-L-Mali-1 (malate-1, also designated as Mali-1). As determined by proton NMR, the stoichiometric ratio of base to acid was 1:1.

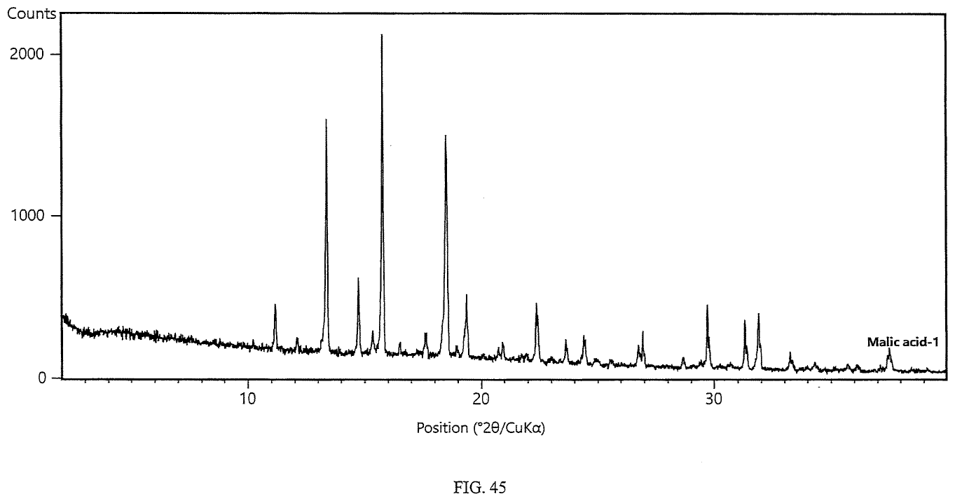

The Mali-1 obtained was analyzed by XRPD. The XRPD diffractogram of Mali-1 is depicted in FIG. 45. The peak values and intensity of the XPRD of the Mali-1 salt are provided below in Table 29.

TABLE 29
XRPD peak values for FIG. 45 (Mali-1).

| Peak # | Pos. [°2θ] | Intensity |
|---|---|---|
| 1 | 11.1721 | 263.82 |
| 2 | 12.1059 | 67.66 |
| 3 | 13.3822 | 1374.07 |
| 4 | 14.7302 | 456.69 |
| 5 | 15.3409 | 122.29 |
| 6 | 15.7688 | 1930.08 |
| 7 | 16.4979 | 70.44 |
| 8 | 17.6293 | 116.18 |
| 9 | 18.4924 | 1317.74 |
| 10 | 18.9377 | 58.50 |
| 11 | 19.3630 | 375.73 |
| 12 | 20.9303 | 96.92 |
| 13 | 22.3519 | 316.01 |
| 14 | 23.6187 | 140.13 |
| 15 | 24.4159 | 125.50 |
| 16 | 24.9386 | 23.34 |
| 17 | 26.7272 | 108.90 |
| 18 | 26.9167 | 159.22 |
| 19 | 28.6489 | 58.21 |
| 20 | 29.4632 | 40.35 |
| 21 | 29.6955 | 370.71 |
| 22 | 31.2940 | 283.92 |
| 23 | 31.8862 | 338.13 |
| 24 | 33.2404 | 107.60 |
| 25 | 34.3110 | 34.06 |
| 26 | 35.7117 | 35.48 |
| 27 | 36.1274 | 29.99 |
| 28 | 37.5101 | 129.37 |

As shown, in an embodiment, this Mali-1 salt can be characterized by peaks in the X-Ray diffractogram at 13.4, 15.8, and 18.5±0.5 ° 2θ. In another embodiment, this salt can be characterized by peaks at 13.4, 14.7, 15.8, 18.5, and 19.4±0.5 ° 2θ. In another embodiment, this salt can be characterized by peaks at 11.2, 13.4, 14.7, 15.8, 18.5, 19.4, 22.4, 29.7, 31.3, and 31.9±0.5 ° 2θ. In another embodiment, this salt can be characterized by peaks at 11.2, 12.1, 13.4, 14.7, 15.3, 15.8, 16.5, 17.6, 18.5, 18.9, 19.4, 20.9, 22.4, 23.6, 24.4, 24.9, 26.7, 26.9, 28.6, 29.4, 29.7, 31.3, 31.9, 33.2, 34.3, 35.7, 36.1, and 37.5±0.5 ° 2θ.

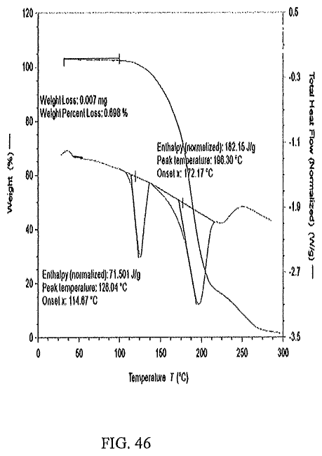

The Mali-1 salt was analyzed by DSC-TGA using the methodology described hereinabove. The data is depicted in FIG. 46. There was a weight loss of about 0.70% when the temperature was raised from room temperature to 100 °C. In addition, the DSC thermogram of the Mali-1 salt exhibited two temperature peaks, a broad temperature peak at about 196.3 °C and a sharp temperature peak at about 126.0 °C.

Substituting deuterated R freebase for freebase (R)-2-(4-fluorophenyl)-2-(methylamino)cyclohexan-1-one in the reaction with L-malic acid produces a crystalline polymorph salt of deuterated (R)-2-(4-fluorophenyl)-2-(methylamino)cyclohexan-1-one malate-1 (deuterated R malate-1, also identified as deuterated R-Mali-1). The XRPD diffractogram and DSC-TGA of deuterated R-Mali-1 are substantially the same as the XRPD diffractogram and DSC-TGA, respectively of Mali-1. The DVS pattern of the deuterated R-Mali-1 is substantially the same as that of Mali-1.

2. (S)-2-(4-fluorophenyl)-2-(methylamino)cyclohexan-1-one-D-malate and its deuterated analogs

Substituting S freebase and deuterated S freebase for freebase (R)-2-(4-fluorophenyl)-2-(methylamino)cyclohexan-1-one and D-malic acid for L-malic acid in the reaction described hereinabove produces a crystalline polymorph salt of(S)-2-(4-fluorophenyl)-2-(methylamino)cyclohexan-1-one D-malate-1 (also identified as S Mali-1) and deuterated(S)-2-(4-fluorophenyl)-2-(methylamino)cyclohexan-1-one D-malate-1 (deuterated S malate-1, also identified as deuterated S Mali-1), respectively. The XRPD diffractogram and DSC-TGA of S Mali-1 and deuterated S Mali-1 are substantially the same as the XRPD diffractogram and DSC-TGA, respectively of Mali-1. The DVS patterns both the S Mali-1 and deuterated S Mali-1 are substantially the same as that of Mali-1.

(1) 1. (R)-2-(4-fluorophenyl)-2-(methylamino)cyclohexan-1-one hippurate and its deuterated analog

Isopropanol, ethyl acetate, and tetrahydrofuran (0.25 mL of each solvent) were added to three different glass vials containing about 40 mg of freebase (R)-2-(4-fluorophenyl)-2-(methylamino)cyclohexan-1-one. One equivalent of hippuric acid was added to each vial and the contents were stirred at room temperature. A solid product was observed in each and was collected from each and vacuum dried at room temperature. As determined by XRPD, the products isolated from each of the vials were crystalline and all exhibited the same XRPD diffraction pattern. This product will be identified herein as (R)-2-(4-fluorophenyl)-2-(methylamino)cyclohexan-1-one Hip-1 (hippurate-1, also designated as Hip-1). As determined by proton NMR, the stoichiometric ratio of base to acid was 1:1.

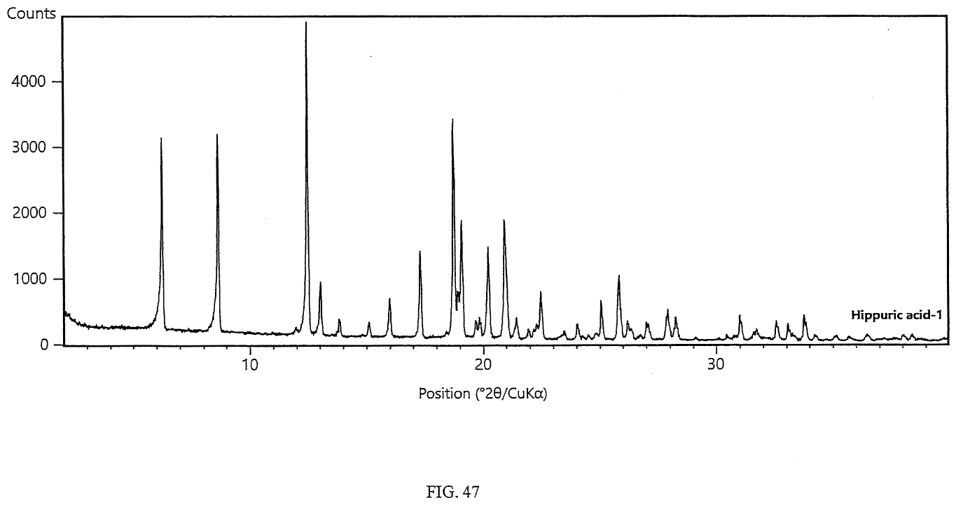

The Hip-1 obtained was analyzed by XRPD. The XRPD diffractogram of Hip-1 is depicted in FIG. 47. The peak values and intensity of the XPRD of the Hip-1 salt are provided below in Table 30.

TABLE 30
XRPD peak values for FIG. 47 (Hip-1).

| Peak # | Pos. [°2θ] | Intensity |
|---|---|---|
| 1 | 6.2305 | 2211.45 |
| 2 | 8.6362 | 2257.64 |
| 3 | 12.4614 | 3087.53 |
| 4 | 13.0098 | 762.47 |
| 5 | 13.8322 | 163.90 |
| 6 | 15.0865 | 205.62 |
| 7 | 15.9754 | 437.85 |
| 8 | 17.2922 | 854.62 |
| 9 | 18.7236 | 1929.06 |
| 10 | 18.8918 | 692.28 |
| 11 | 19.0590 | 1370.20 |
| 12 | 19.6755 | 171.47 |
| 13 | 19.8315 | 293.95 |
| 14 | 20.2045 | 1085.04 |
| 15 | 20.9043 | 1380.78 |
| 16 | 20.9821 | 1102.20 |
| 17 | 21.4090 | 214.57 |
| 18 | 21.9261 | 86.91 |
| 19 | 22.2102 | 106.70 |
| 20 | 22.4489 | 476.50 |
| 21 | 23.4805 | 78.96 |
| 22 | 24.0299 | 138.50 |
| 23 | 24.5032 | 56.96 |
| 24 | 25.0452 | 270.99 |
| 25 | 25.8273 | 672.63 |
| 26 | 26.1795 | 233.65 |
| 27 | 26.3063 | 102.45 |
| 28 | 26.9773 | 212.90 |
| 29 | 27.8778 | 253.64 |
| 30 | 28.2464 | 212.39 |
| 31 | 29.1055 | 15.85 |
| 32 | 30.4384 | 54.57 |
| 33 | 30.8096 | 78.09 |
| 34 | 30.9916 | 231.09 |
| 35 | 31.7061 | 103.25 |
| 36 | 32.5413 | 194.27 |
| 37 | 33.0512 | 150.10 |
| 38 | 33.1714 | 67.49 |
| 39 | 33.7479 | 233.31 |
| 40 | 33.8534 | 154.87 |
| 41 | 34.2352 | 33.93 |
| 42 | 35.1513 | 36.72 |
| 43 | 35.7076 | 23.37 |
| 44 | 36.4758 | 44.93 |
| 45 | 38.0252 | 60.99 |
| 46 | 38.3701 | 55.99 |

As shown, in an embodiment, this Hip-1 salt can be characterized by intense peaks in the X-Ray diffractogram at 6.2, 8.6, and 12.5±0.5 ° 2θ. In another embodiment, this salt can be characterized by peaks at 6.2, 8.6, 12.5, 18.7, and 20.9±0.5 ° 2θ. In another embodiment, this salt can be characterized by peaks at 6.2, 8.6, 12.5, 13.0, 17.3, 18.7, 19.1, 20.2, 20.9, and 21.0±0.5 ° 2θ. In another embodiment, this salt can be characterized by peaks at 6.2, 8.6, 12.5, 13.0, 13.8, 15.1, 16.0, 17.3, 18.7, 18.9, 19.1, 19.7, 19.8, 20.2, 20.9, 21.0, 21.4, 21.9, 22.2, 22.4, 23.5, 24.0.5, 24.5, 25.0, 25.8, 26.2, 26.3, 27.0, 27.9, 28.2, 29.1, 30.4, 30.8, 31.0, 31.7, 32.5, 33.1, 33.2, 33.7, 33.9, 34.2, 35.2, 35.7, 36.5, 38.0, and 38.4±0.5 ° 2θ.

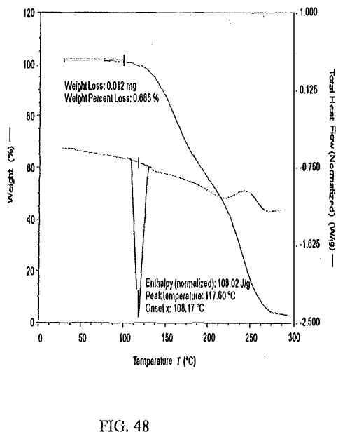

The Hip-1 salt was analyzed by DSC-TGA using the methodology described hereinabove. The data is depicted in FIG. 48. There was a weight loss of about 0.69% when the temperature was raised from room temperature to 100 °C. In addition, the DSC thermogram of the Hip-1 salt exhibited an intense peak temperature at about 117.6 °C.

Substituting deuterated R freebase for freebase (R)-2-(4-fluorophenyl)-2-(methylamino)cyclohexan-1-one in the reaction with hippuric acid produces deuterated (R)-2-(4-fluorophenyl)-2-(methylamino)cyclohexan-1-one hippurate-1 (deuterated R hippurate-1, also identified as deuterated R Hip-1). The XRPD diffractogram and the DSC-TGA of deuterated R Hip-1 are substantially the same as the XRPD diffractogram and the DSC-TGA of Hip-1. Further, the DVS pattern of deuterated R Hip-1 salt is substantially similar to that of Hip-1.

2. (S)-2-(4-fluorophenyl)-2-(methylamino)cyclohexan-1-one hippurate and its deuterated analog. Substituting S freebase and deuterated S freebase for freebase (R)-2-(4-fluorophenyl)-2-(methylamino)cyclohexan-1-one in the reaction with hippuric acid described above produces(S)-2-(4-fluorophenyl)-2-(methylamino)cyclohexan-1-one hippurate-1 (S-hippurate-1, also identified as S Hip-1) and deuterated(S)-2-(4-fluorophenyl)-2-(methylamino)cyclohexan-1-one hippurate-1 (deuterated S hippurate-1, also identified as deuterated S Hip-1) respectively. The XRPD diffractogram and the DSC-TGA of both S Hip-1 and deuterated S Hip-1 are substantially the same as the XRPD diffractogram and the DSC-TGA of Hip-1. Further, the DVS patterns of the S Hip-1 and deuterated S Hip-1 salt are substantially the same as that of Hip-1.

### Utilities and Pharmaceutical Compositions

The salts disclosed herein are useful for treating psychiatric disorders. The utility is the same as described in U.S. Pat. No. 11,344,510, the contents of which are incorporated by reference.

More specifically, in another aspect, provided herein is a method of treating depression or anxious depression in a subject in need thereof, the method comprising administering to the subject in need thereof an effective amount of a salt disclosed herein. In some embodiments, the salts are orally administered.

The salts are useful in a method for treating a psychiatric disorder in a patient in need of treatment, such method comprising administering to a subject in need thereof a therapeutically effective amount of a salt or composition comprising a salt disclosed herein. Contemplated psychiatric disorders may include Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, and Depressive Disorder Due to Another Medical Condition. The salts described herein are useful for treating refractory depression, e.g., patients suffering from a depressive disorder that does not, and/or has not, responded to adequate courses of at least one, or at least two, other antidepressant compounds or therapeutics. As used herein “depressive disorder” encompasses refractory depression. In some embodiments, the salts disclosed herein may be used to treat a psychiatric disorder including Bipolar and Related Disorders, e.g., Bipolar I Disorder, Bipolar II Disorder, Cyclothymic Disorder, Substance/Medication-Induced Bipolar and Related Disorder, and Bipolar and Related Disorders due to another medical condition. In some embodiments, the salts disclosed herein may be used to treat a psychiatric disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine, and tobacco. As used herein “substance” or “substances” are psychoactive compounds which can be addictive, such as alcohol, caffeine, cannabis, hallucinogens, inhalants, opioids, sedatives, hypnotics, anxiolytics, stimulants, nicotine, and tobacco. For example, the methods and compositions may be used to facilitate smoking cessation or cessation of opioid use.

In some embodiments, the salts disclosed herein may be used to treat a psychiatric disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition. In embodiments, the salts disclosed herein may be used to treat a psychiatric disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive, and Related Disorder, and Obsessive-Compulsive and Related Disorder Due to Another Medical Condition. In some embodiments, the salts disclosed herein may be used to treat a psychiatric disorder including Trauma- and Stressor-Related Disorders, e.g., Reactive Attachment Disorder, Disinhibited Social Engagement Disorder, Posttraumatic Stress Disorder, Acute and Stress Disorder, and Adjustment Disorders. In some embodiments, the salts disclosed herein may be used to treat a psychiatric disorder including Feeding and Eating Disorders, e.g., Anorexia Nervosa, Bulimia Nervosa, Binge-Eating Disorder, Pica, Rumination Disorder, and Avoidant/Restrictive Food Intake Disorder.

Further, in some embodiments, the salts disclosed herein may be used to treat a psychiatric disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies. Moreover, in some embodiments, the salts disclosed herein may be used to treat a psychiatric disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder. Further, in some embodiments, the salts disclosed herein may be used to treat a psychiatric disorder including Personality Disorders, e.g., Borderline Personality Disorder.

In addition, in some embodiments, the salts disclosed herein may be used to treat a psychiatric disorder including Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction. In some embodiments the salts disclosed herein may be used to treat a psychiatric disorder including Gender Dysphoria, e.g., Gender Dysphoria.

In some embodiments, methods of using the salts disclosed herein include treating a psychiatric disorder by administering to a subject in need thereof a pharmaceutical composition including about 0.01 mg to about 400 mg of a salt disclosed herein. In some embodiments, doses may be, e.g., in the range of about 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 300 mg, 10 to 250 mg, 10 to 200 mg, 10 to 150 mg, 10 to 100 mg, 10 to 50 mg, 10 to 25 mg, 10 to 15 mg, 20 to 300 mg, 20 to 250 mg, 20 to 200 mg, 20 to 150 mg, 20 to 100 mg, 20 to 50 mg, 50 to 300 mg, 50 to 250 mg, 50 to 200 mg, 50 to 150 mg, 50 to 100 mg, 100 to 300 mg, 100 to 250 mg, 100 to 200 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In some embodiments, dosages may include amounts of the salts disclosed herein in the range of about, e.g., 1 mg to 200 mg, 1 mg to 100 mg, 1 mg to 50 mg, 1 mg to 40 mg, 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, 30 mg, 35 mg, 40 mg, 45 mg, 50 mg, 60 mg, 75 mg, 80 mg, 90 mg, 100 mg, 125 mg, 150 mg, and 200 mg being specific examples of doses.

Typically, dosages of a salt disclosed herein are administered once, twice, three or four times daily, every other day, every three days, once weekly, or once a month to a patient in need thereof. In some embodiments, the dosage is about, e.g., 1-400 mg/day, or 1-300 mg/day, or 1-250 mg/day, or 1-200 mg/day, for example 300 mg/day, 250 mg/day, 200 mg/day, 150 mg/day, 100 mg/day, 75 mg/day, 50 mg/day, 25 mg/day, 20 mg/day, 10 mg/day, 5 mg/day, or 1 mg/day.

In some embodiments, pharmaceutical compositions for parenteral administration or inhalation, e.g., a spray or mist of salt disclosed herein, include a concentration of about 0.005 mg/mL to about 500 mg/mL. In some embodiments, the compositions include a salt disclosed herein at a concentration of, e.g., about 0.05 mg/mL to about 50 mg/mL, about 0.05 mg/mL to about 100 mg/mL, about 0.005 mg/mL to about 500 mg/mL, about 0.1 mg/mL to about 50 mg/mL, about 0.1 mg/mL to about 10 mg/mL, about 0.05 mg/mL to about 25 mg/mL, about 0.05 mg/mL to about 10 mg/mL, about 0.05 mg/mL to about 5 mg/mL, or about 0.05 mg/mL to about 1 mg/mL.

In some embodiments, the composition includes a salt disclosed herein at a concentration of, e.g., about 0.05 mg/mL to about 15 mg/mL, about 0.5 mg/mL to about 10 mg/mL, about 0.25 mg/mL to about 5 mg/mL, about 0.5 mg/mL to about 7 mg/mL, about 1 mg/mL to about 10 mg/mL, about 5 mg/mL to about 10 mg/mL, about 5 mg/mL to about 15 mg/mL, about 5 mg/mL to 25 mg/mL, about 5 mg/mL to 50 mg/mL, about 10 mg/mL to 100 mg/mL, about 20 mg/mL to 200 mg/mL, or about 30 mg/mL to about 300 mg/mL. In some embodiments, the pharmaceutical compositions are formulated as a total volume of about, e.g., 10 mL, 20 mL, 25 mL, 50 mL, 100 mL, 200 mL, 250 mL, or 500 mL.

Typically, dosages may be administered to a subject once, twice, three or four times daily, every other day, every three days, twice weekly, once weekly, twice monthly, or once monthly. In some embodiments, a salt disclosed herein is administered to a subject once in the morning, or once in the evening. In some embodiments, a salt disclosed herein is administered to a subject once in the morning, and once in the evening. In some embodiments, a salt disclosed herein is administered to a subject three times a day (e.g., at breakfast, lunch, and dinner), at a dose, e.g., of 50 mg/administration (e.g., 150 mg/day).

In some embodiments, a salt disclosed herein is administered to a subject at a dose of 25 mg/day in one or more doses. In some embodiments, a salt disclosed herein is administered to a subject at a dose of 50 mg/day in one or more doses. In some embodiments, a salt disclosed herein is administered to a subject at a dose of 100 mg/day in one or more doses. In some embodiments, a salt disclosed herein is administered to a subject at a dose of 150 mg/day in one or more doses. In some embodiments, a salt disclosed herein is administered to a subject at a dose of 200 mg/day in one or more doses. In some embodiments, a salt disclosed herein is administered to a subject at a dose of 250 mg/day in one or more doses.

In some embodiments, the dosage of a salt disclosed herein is 0.01-100 mg/kg, 0.5-50 mg/kg, 0.5-10 mg/kg, or 25-50 mg/kg once, twice, three times or four times daily. For example, in some embodiments, the dosage is 0.1 mg/kg, 0.25 mg/kg, 0.5 mg/kg, 1 mg/kg, 5 mg/kg, 7.5 mg/kg, or 10 mg/kg once, twice, three times or four times daily. In some embodiments, a subject is administered a total daily dose of 0.01 mg to 500 mg of a salt disclosed herein once, twice, three times, or four times daily. In some embodiments, the total amount administered to a subject in a 24-hour period is, e.g., 5 mg, 10 mg, 20 mg, 25 mg, 30 mg, 35 mg, 40 mg, 45 mg, 50 mg, 60 mg, 75 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg. In some embodiments, the subject may be started at a low dose and the dosage is escalated. In some embodiments, the subject may be started at a high dose and the dosage is decreased.

In some embodiments, a salt disclosed herein is administered to a patient under the supervision of a healthcare provider.

In some embodiments, a salt disclosed herein is administered to a patient under the supervision of a healthcare provider at a clinic specializing in the delivery of psychoactive treatments.

In some embodiments, a salt disclosed herein, is administered to a patient under the supervision of a healthcare provider at a dose intended to induce a psychedelic experience in the subject.

In some embodiments, the administration to a patient under the supervision of a healthcare provider occurs periodically in order to maintain a therapeutic effect in the patient, e.g., every three days, twice weekly, once weekly, twice monthly, once monthly, thrice yearly, twice yearly, or once yearly.

In some embodiments, a salt disclosed herein, is administered by a patient on their own at home or otherwise away from the supervision of a healthcare provider.

In some embodiments, the administration by a patient on their own occurs periodically in order to maintain a therapeutic effect in the patient, e.g., daily, every other day, every three days, twice weekly, once weekly, twice monthly, or once monthly.

In some embodiments, a salt disclosed herein may be administered at specified intervals. For example, during treatment a patient may be administered a salt disclosed herein at intervals of every, e.g., 1 year, 6 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In some embodiments, a pharmaceutical composition comprises one or more of the salts disclosed herein. In some embodiments, a salt disclosed herein is used in any of the methods, uses, or compositions described herein.

The present disclosure thus also relates to pharmaceutical compositions comprising a salt disclosed herein in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal, and sublingual), vaginal, or parenteral (including subcutaneous, intramuscular, intravenous, and intradermal) administration or administration via an implant. The compositions may be prepared by any method well known in the art of pharmacy.

Such methods include the step of bringing in association a salt disclosed herein with any auxiliary agent. The auxiliary agent(s), also include accessory ingredient(s), include those conventional in the art, such as carriers, fillers, binders, diluents, disintegrants, lubricants, colorants, flavoring agents, anti-oxidants, and wetting agents. Such auxiliary agents are suitably selected with respect to the intended form and route of administration and as consistent with conventional pharmaceutical practices.

Pharmaceutical compositions suitable for oral administration may be presented as discrete dosage units such as pills, tablets, dragées or capsules, or as a powder or granules, or as a solution or suspension. The active ingredient comprised of a salt disclosed herein may also be presented as a bolus or paste. The compositions can further be processed into a suppository or enema for rectal administration.

The salts in a pharmaceutical composition may be administered in a solid dosage form. For example, tablets may contain as the active ingredient, a salt disclosed in association with suitable binders, lubricants, disintegrating agents, coloring agents, flavoring agents, flow-inducing agents, and melting agents. Gelatin capsules may contain the active ingredient salt disclosed herein and powdered carriers, such as lactose, starch, cellulose derivatives, magnesium stearate, stearic acid, and the like. Similar diluents can be used to make compressed tablets. Compressed tablets can be sugar coated or film coated to mask any unpleasant taste and protect the tablet from the atmosphere, or enteric coated for selective disintegration in the gastrointestinal tract. For instance, for oral administration in the dosage unit form of a tablet or capsule, the active drug component comprised of a salt disclosed herein can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

For oral administration in liquid dosage form, one or more salts disclosed herein, are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols, or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration can contain coloring and flavoring to increase patient acceptance.

For parenteral administration, suitable compositions include aqueous and non-aqueous sterile solutions comprised of one or more salts disclosed herein. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, i.e., a salt disclosed herein, and suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g., gels, patches or sprays can be contemplated. Compositions or formulations suitable for pulmonary administration e.g., by nasal inhalation, include fine dusts or mists which may be generated by means of metered dose pressurized aerosols, nebulizers, or insufflators. Parenteral and intravenous forms may also include minerals and other materials to make them compatible with the type of injection or delivery system chosen.

The salts disclosed herein used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The salts disclosed herein may be administered as components of tissue-targeted emulsions.

The salts disclosed herein used in the method of the present disclosure may also be coupled to soluble polymers as targetable drug carriers or as prodrugs. Such polymers include polyvinylpyrrolidone, pyran copolymer, polyhydroxylpropylmethacrylamide-phenol, polyhydroxyethylaspartamide-phenol, or polyethyleneoxide-polylysine substituted with palmitoyl residues. Furthermore, the salts disclosed herein may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Pharmaceutical compositions herein may be provided with immediate release, delayed release, extended release, or modified release profiles. In some embodiments, pharmaceutical compositions with different drug release profiles may be combined to create a two-phase or three-phase release profile. For example, pharmaceutical compositions may be provided with an immediate release and an extended-release profile. Such composition may be provided as pulsatile formulations, multilayer tablets, or capsules containing tablets, beads, granules, etc.

Pharmaceutical compositions herein may be provided with abuse deterrent features by techniques known in the art, for example, by making a tablet that is difficult to crush or to dissolve in water.

The pharmaceutical composition, as hereinbefore described, may be in combination with packaging material, including instructions for the use of the composition for a use as hereinbefore described.

The exact dose and regimen of administration of the composition comprised of salts disclosed herein, will necessarily be dependent upon the type and magnitude of the therapeutic or nutritional effect to be achieved and may vary depending on factors such as the particular compound, formula, route of administration, or age and condition of the individual subject to whom the composition is to be administered.

Furthermore, in some embodiments, a pharmaceutical composition disclosed herein may include a single enantiomer, diastereomer or structural isomer of a salt disclosed herein, where such stereoisomers exist. In other embodiments, a pharmaceutical composition disclosed herein may include a mixture of at least one single enantiomer, diastereomer or structural isomer of a salt disclosed herein. together with another enantiomer, diastereomer or structural isomer of a salt disclosed herein. In further embodiments, said mixture is a racemic mixture. In other embodiments, said mixture is a non-racemic mixture (wherein one enantiomer or diastereomer is enriched in said non-racemic mixture). In some embodiments, the salts disclosed herein are substantially pure, or are enantiomerically pure, or both, or may contain polymorphs of any of the salts disclosed herein.

The salts disclosed herein may be administered in various forms, including those detailed herein. The treatment with the salt disclosed may be a component of a combination therapy or an adjunct therapy, i.e., the subject or patient in need of the drug is treated or given another drug for the disease in conjunction with one or more of the instant salts. This combination therapy can be sequential therapy where the patient is treated first with one drug and then the other or the two drugs can be given simultaneously. These can be administered independently by the same route or by two or more different routes of administration depending on the dosage forms employed.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A crystalline salt of (R)-2-(4-fluorophenyl)-2-(methylamino)cyclohexan-1-one comprising crystalline (R)-2-(4-fluorophenyl)-2-(methylamino)cyclohexan-1-one hydrochloride salt, crystalline (R)-2-(4-fluorophenyl)-2-(methylamino)cyclohexan-1-one sulfate salt, crystalline (R)-2-(4-fluorophenyl)-2-(methylamino)cyclohexan-1-one p-toluenesulfonate salt, crystalline (R)-2-(4-fluorophenyl)-2-(methylamino)cyclohexan-1-one methanesulfonate salt, crystalline (R)-2-(4-fluorophenyl)-2-(methylamino)cyclohexan-1-one maleate salt, crystalline (R)-2-(4-fluorophenyl)-2-(methylamino)cyclohexan-1-one phosphate salt, crystalline (R)-2-(4-fluorophenyl)-2-(methylamino)cyclohexan-1-one tartrate salt, crystalline (R)-2-(4-fluorophenyl)-2-(methylamino)cyclohexan-1-one fumarate salt, crystalline (R)-2-(4-fluorophenyl)-2-(methylamino)cyclohexan-1-one citrate salt, crystalline (R)-2-(4-fluorophenyl)-2-(methylamino)cyclohexan-1-one malate salt, or crystalline (R)-2-(4-fluorophenyl)-2-(methylamino)cyclohexan-1-one hippurate salt.

2. The crystalline (R)-2-(4-fluorophenyl)-2-(methylamino)cyclohexan-1-one hydrochloride salt of claim 1, which is crystalline (R)-2-(4-fluorophenyl)-2-(methylamino)cyclohexan-1-one hydrochloride Form 1 salt characterized by an X-ray powder diffraction pattern comprising peaks at 9.7, 15.0, and 18.6±0.5 ° 2θ.

3. (canceled)

4. (canceled)

5. The crystalline (R)-2-(4-fluorophenyl)-2-(methylamino)cyclohexan-1-one hydrochloride Form 1 salt of claim 2 characterized by an X-ray powder diffraction pattern comprising peaks at 9.7, 13.1, 15.0, 18.6, and 24.2±0.5 ° 2θ.

6. The crystalline (R)-2-(4-fluorophenyl)-2-(methylamino)cyclohexan-1-one hydrochloride Form 1 salt of claim 2 characterized by an X-ray powder diffraction pattern comprising peaks at 9.7, 13.1, 14.6, 15.0, 18.6, 19.2, 22.5, 24.2, 25.1, and 31.6±0.5 ° 2θ.

7. (canceled)

8. (canceled)

9. The crystalline (R)-2-(4-fluorophenyl)-2-(methylamino)cyclohexan-1-one hydrochloride Form 1 salt of claim 2 characterized by a DSC thermogram having at least one endotherm with a peak at about 238 °C.

10. (canceled)

11. The crystalline (R)-2-(4-fluorophenyl)-2-(methylamino)cyclohexan-1-one hydrochloride salt of claim 1 which is crystalline (R)-2-(4-fluorophenyl)-2-(methylamino)cyclohexan-1-one hydrochloride Form 2 salt characterized by an X-ray powder diffraction pattern comprising peaks at 13.2, 15.0, and 24.0±0.5 ° 2θ.

12. (canceled)

13. The crystalline (R)-2-(4-fluorophenyl)-2-(methylamino)cyclohexan-1-one hydrochloride Form 2 salt of claim 11 characterized by an X-ray powder diffraction pattern comprising peaks at 13.2, 15.0, 18.4, 18.7, and 24.0±0.5 ° 2θ.

14. The crystalline (R)-2-(4-fluorophenyl)-2-(methylamino)cyclohexan-1-one hydrochloride Form 2 salt of claim 11 characterized by an X-ray powder diffraction pattern comprising peaks at 13.0, 13.2, 15.0, 17.2, 18.4, 18.7, 24.0, 26.9, 27.1, and 31.1±0.5 ° 2θ.

15. (canceled)

16. The crystalline (R)-2-(4-fluorophenyl)-2-(methylamino)cyclohexan-1-one sulfate salt of claim 1, which is crystalline (R)-2-(4-fluorophenyl)-2-(methylamino)cyclohexan-1-one sulfate-1 salt, characterized by an X-ray powder diffraction pattern comprising peaks at 10.2, 16.0, and 17.6±0.5 ° 2θ.

17. (canceled)

18. (canceled)

19. The crystalline (R)-2-(4-fluorophenyl)-2-(methylamino)cyclohexan-1-one sulfate-1 salt of claim 16 characterized by an X-ray powder diffraction pattern comprising peaks at 10.2, 16.0, 16.4, 17.6, and 22.7±0.5° 2θ.

20. (canceled)

21. (canceled)

22. (canceled)

23. The crystalline (R)-2-(4-fluorophenyl)-2-(methylamino)cyclohexan-1-one sulfate-1 salt of claim 16 characterized by a DSC thermogram having at least one endotherm with a peak at about 221.3 °C.

24. (canceled)

25. (canceled)

26. The crystalline (R)-2-(4-fluorophenyl)-2-(methylamino)cyclohexan-1-one sulfate salt of claim 1, which is crystalline (R)-2-(4-fluorophenyl)-2-(methylamino)cyclohexan-1-one sulfate-3 salt characterized by an X-ray powder diffraction pattern comprising peaks at 11.9, 16.0, and 18.7±0.5 ° 2θ.

27. (canceled)

28. The crystalline (R)-2-(4-fluorophenyl)-2-(methylamino)cyclohexan-1-one sulfate-3 salt of claim 26 characterized by an X-ray powder diffraction pattern comprising peaks at 10.2, 11.9, 16.0, 17.6, and 18.7±0.5° 2θ.

29. (canceled)

30. (canceled)

31. (canceled)

32. The crystalline (R)-2-(4-fluorophenyl)-2-(methylamino)cyclohexan-1-one sulfate salt of claim 1, which is crystalline (R)-2-(4-fluorophenyl)-2-(methylamino)cyclohexan-1-one sulfate-4 salt characterized by an X-ray powder diffraction pattern comprising peaks at 7.9, 16.0, and 17.6±0.5 ° 2θ.

33. (canceled)

34. The crystalline (R)-2-(4-fluorophenyl)-2-(methylamino)cyclohexan-1-one sulfate-4 salt of claim 32 characterized by an X-ray powder diffraction pattern comprising peaks at 7.9, 10.2, 16.0, 17.6, and 22.7±0.5 ° 2θ.

35. (canceled)

36. (canceled)

37. (canceled)

38. The crystalline (R)-2-(4-fluorophenyl)-2-(methylamino)cyclohexan-1-one p-toluenesulfonate salt of claim 1, which is (R)-2-(4-fluorophenyl)-2-(methylamino)cyclohexan-1-one p-toluenesulfonate-1 salt characterized by an X-ray powder diffraction pattern comprising peaks at 6.6, 10.6, and 19.5±0.5 ° 2θ.

39. (canceled)

40. (canceled)

41. The crystalline (R)-2-(4-fluorophenyl)-2-(methylamino)cyclohexan-1-one p-toluenesulfonate-1 salt of claim 38 characterized by an X-ray powder diffraction pattern comprising peaks at 6.6, 10.6, 13.3, 14.6, and 19.5±0.5 ° 2θ.

42. (canceled)

43. (canceled)

44. (canceled)

45. The crystalline (R)-2-(4-fluorophenyl)-2-(methylamino)cyclohexan-1-one p-toluenesulfonate-1 salt of claim 38 characterized by a DSC thermogram having at least one endotherm with a peak at about 156.9 °C.

46. (canceled)

47. (canceled)

48. The crystalline (R)-2-(4-fluorophenyl)-2-(methylamino)cyclohexan-1-one p-toluenesulfonate salt of claim 1, which is (R)-2-(4-fluorophenyl)-2-(methylamino)cyclohexan-1-one p-toluenesulfonate-2B, characterized by an X-ray powder diffraction pattern comprising peaks at 5.4, 19.6, and 21.6±0.5 ° 2θ.

49. (canceled)

50. The crystalline (R)-2-(4-fluorophenyl)-2-(methylamino)cyclohexan-1-one p-toluenesulfonate-2B salt of claim 48 characterized by an X-ray powder diffraction pattern comprising peaks at 5.4, 10.7, 16.1, 19.6, and 21.6±0.5 ° 2θ.

51. (canceled)

52. (canceled)

53. The crystalline (R)-2-(4-fluorophenyl)-2-(methylamino)cyclohexan-1-one p-toluenesulfonate-2B salt of claim 48 characterized by a DSC thermogram having at least one endotherm with a peak at about 168.7 °C.

54. (canceled)

55. (canceled)

56. (canceled)

57. The crystalline (R)-2-(4-fluorophenyl)-2-(methylamino)cyclohexan-1-one methanesulfonate salt of claim 1 characterized by an X-ray powder diffraction pattern comprising peaks at 10.1, 15.7, and 16.9±0.5 ° 2θ.

58. (canceled)

59. The crystalline (R)-2-(4-fluorophenyl)-2-(methylamino)cyclohexan-1-one methanesulfonate salt of claim 57 characterized by an X-ray powder diffraction pattern comprising peaks at 10.1, 15.7, 16.9, 22.2, and 22.6±0.5 ° 2θ.

60. (canceled)

61. (canceled)

62. (canceled)

63. The crystalline (R)-2-(4-fluorophenyl)-2-(methylamino)cyclohexan-1-one methanesulfonate salt of claim 57 characterized by a DSC thermogram having at least one endotherm with a peak at about 228.3 °C.

64. (canceled)

65. The crystalline (R)-2-(4-fluorophenyl)-2-(methylamino)cyclohexan-1-one maleate salt of claim 1 which is crystalline (R)-2-(4-fluorophenyl)-2-(methylamino)cyclohexan-1-one maleate-1 salt characterized by an X-ray powder diffraction pattern comprising peaks at 16.2, 19.4, and 24.6±0.5 ° 2θ.

66. (canceled)

67. (canceled)

68. The crystalline (R)-2-(4-fluorophenyl)-2-(methylamino)cyclohexan-1-one maleate-1 salt of claim 65 characterized by an X-ray powder diffraction pattern comprising peaks at 14.3, 16.2, 19.4, 24.6, and 24.7±0.5 ° 2θ.

69. (canceled)

70. (canceled)

71. (canceled)

72. The crystalline (R)-2-(4-fluorophenyl)-2-(methylamino)cyclohexan-1-one maleate-1 salt of claim 65 characterized by a DSC thermogram having at least one endotherm with a peak at about 202.0 °C.

73. (canceled)

74. The crystalline (R)-2-(4-fluorophenyl)-2-(methylamino)cyclohexan-1-one maleate salt of claim 1, which is crystalline (R)-2-(4-fluorophenyl)-2-(methylamino)cyclohexan-1-one maleate-2 salt characterized by an X-ray powder diffraction pattern comprising peaks at 13.3, 16.0, and 18.8±0.5 ° 2θ.

75. (canceled)

76. The crystalline (R)-2-(4-fluorophenyl)-2-(methylamino)cyclohexan-1-one maleate-2 salt of claim 74 characterized by an X-ray powder diffraction pattern comprising peaks at 13.3, 14.7, 16.0, 18.8, and 19.2±0.5 ° 2θ.

77. (canceled)

78. (canceled)

79. (canceled)

80. The crystalline (R)-2-(4-fluorophenyl)-2-(methylamino)cyclohexan-1-one maleate-2 salt of claim 74 characterized by a DSC thermogram having at least one endotherm with a peak at about 198.2 °C.

81. (canceled)

82. The crystalline (R)-2-(4-fluorophenyl)-2-(methylamino)cyclohexan-1-one phosphate salt of claim 1, which is crystalline (R)-2-(4-fluorophenyl)-2-(methylamino)cyclohexan-1-one phosphate-1 salt characterized by an X-ray powder diffraction pattern comprising peaks at 6.6, 19.1, 21.0, and 24.1±0.5 ° 2θ.

83. (canceled)

84. (canceled)

85. The crystalline (R)-2-(4-fluorophenyl)-2-(methylamino)cyclohexan-1-one phosphate-1 salt of claim 82 characterized by an X-ray powder diffraction pattern comprising peaks at 6.6, 15.1, 19.1, 21.0, and 24.1±0.5 ° 2θ.

86. (canceled)

87. (canceled)

88. (canceled)

89. The crystalline (R)-2-(4-fluorophenyl)-2-(methylamino)cyclohexan-1-one phosphate-1 salt of claim 82 characterized by a DSC thermogram having at least one endotherm with a peak at about 236.0 °C.

90. (canceled)

91. The crystalline (R)-2-(4-fluorophenyl)-2-(methylamino)cyclohexan-1-one phosphate salt of claim 1, which is crystalline (R)-2-(4-fluorophenyl)-2-(methylamino)cyclohexan-1-one phosphate-2 salt characterized by an X-ray powder diffraction pattern comprising peaks at 6.6, 21.0, 24.1, and 33.5±0.5 ° 2θ.

92. (canceled)

93. The crystalline (R)-2-(4-fluorophenyl)-2-(methylamino)cyclohexan-1-one phosphate-2 salt of claim 91 characterized by an X-ray powder diffraction pattern comprising peaks at 6.6, 13.4, 21.0, 24.1, and 33.5±0.5 ° 2θ.

94. (canceled)

95. (canceled)

96. (canceled)

97. The crystalline (R)-2-(4-fluorophenyl)-2-(methylamino)cyclohexan-1-one phosphate-2 salt claim 91 characterized by a DSC thermogram having at least one endotherm with a peak at about 224.6 °C.

98. (canceled)

99. The crystalline (R)-2-(4-fluorophenyl)-2-(methylamino)cyclohexan-1-one tartrate salt of claim 1, which is crystalline (R)-2-(4-fluorophenyl)-2-(methylamino)cyclohexan-1-one tartrate-1 salt characterized by an X-ray powder diffraction pattern comprising peaks at 8.9, 15.7, and 19.2±0.5 ° 2θ.

100. (canceled)

101. (canceled)

102. The crystalline (R)-2-(4-fluorophenyl)-2-(methylamino)cyclohexan-1-one tartrate-1 salt of claim 99 characterized by an X-ray powder diffraction pattern comprising peaks at 8.9, 15.5, 15.7, 17.5, and 19.2±0.5° 2θ.

103. (canceled)

104. (canceled)

105. (canceled)

106. The crystalline (R)-2-(4-fluorophenyl)-2-(methylamino)cyclohexan-1-one tartrate salt of claim 1, which is crystalline (R)-2-(4-fluorophenyl)-2-(methylamino)cyclohexan-1-one tartrate-5 characterized by an X-ray powder diffraction pattern comprising peaks at 16.6, 19.0, and 21.7±0.5 ° 2θ.

107. (canceled)

108. The crystalline (R)-2-(4-fluorophenyl)-2-(methylamino)cyclohexan-1-one tartrate-5 salt of claim 106 characterized by an X-ray powder diffraction pattern comprising peaks at 9.3, 14.3, 16.6, 19.0, and 21.7±0.5 ° 2θ.

109. (canceled)

110. (canceled)

111. The crystalline (R)-2-(4-fluorophenyl)-2-(methylamino)cyclohexan-1-one tartrate-5 salt of claim 106 characterized by a DSC thermogram having at least one endotherm with a peak at about 163.5 °C or about 187.2 °C.

112. (canceled)

113. (canceled)

114. The crystalline (R)-2-(4-fluorophenyl)-2-(methylamino)cyclohexan-1-one tartrate salt of claim 1, which is crystalline (R)-2-(4-fluorophenyl)-2-(methylamino)cyclohexan-1-one tartrate-3 salt characterized by an X-ray powder diffraction pattern comprising peaks at 16.8, 18.8, and 19.4±0.5 ° 2θ.

115. (canceled)

116. The crystalline (R)-2-(4-fluorophenyl)-2-(methylamino)cyclohexan-1-one tartrate-3 salt of claim 114 characterized by an X-ray powder diffraction pattern comprising peaks at 7.6, 16.8, 18.8, 19.4, and 20.9±0.5 ° 2θ.

117. (canceled)

118. (canceled)

119. (canceled)

120. The crystalline (R)-2-(4-fluorophenyl)-2-(methylamino)cyclohexan-1-one tartrate-3 salt of claim 114 characterized by a DSC thermogram having at least one endotherm with a peak at about 176.6 °C.

121. (canceled)

122. (canceled)

123. The crystalline (R)-2-(4-fluorophenyl)-2-(methylamino)cyclohexan-1-one tartrate salt of claim 1, which is crystalline (R)-2-(4-fluorophenyl)-2-(methylamino)cyclohexan-1-one tartrate-6 salt characterized by an X-ray powder diffraction pattern comprising peaks at 6.6, 13.1, and 14.5±0.5 ° 2θ.

124. (canceled)

125. The crystalline (R)-2-(4-fluorophenyl)-2-(methylamino)cyclohexan-1-one tartrate-6 salt of claim 123 characterized by an X-ray powder diffraction pattern comprising peaks at 6.6, 13.1, 14.5, 15.2, and 24.1±0.5 ° 2θ.

126. (canceled)

127. (canceled)

128. (canceled)

129. The crystalline (R)-2-(4-fluorophenyl)-2-(methylamino)cyclohexan-1-one tartrate-6 salt of claim 123 characterized by a DSC thermogram having at least one endotherm with a peak at about 61.7 °C.

130. (canceled)

131. (canceled)

132. The crystalline (R)-2-(4-fluorophenyl)-2-(methylamino)cyclohexan-1-one fumarate salt of claim 1 which is crystalline (R)-2-(4-fluorophenyl)-2-(methylamino)cyclohexan-1-one fumarate-1 salt characterized by an X-ray powder diffraction pattern comprising peaks at 14.8, 24.8, and 25.7±0.5 ° 2θ.

133. (canceled)

134. (canceled)

135. The crystalline (R)-2-(4-fluorophenyl)-2-(methylamino)cyclohexan-1-one fumarate-1 salt of claim 132 characterized by an X-ray powder diffraction pattern comprising peaks at 7.4, 9.1, 14.8, 24.8, and 25.7±0.5 ° 2θ.

136. (canceled)

137. (canceled)

138. (canceled)

139. The crystalline (R)-2-(4-fluorophenyl)-2-(methylamino)cyclohexan-1-one fumarate-1 salt of claim 132 characterized by a DSC thermogram having at least one endotherm with a peak at about 181.7 °C.

140. (canceled)

141. The crystalline (R)-2-(4-fluorophenyl)-2-(methylamino)cyclohexan-1-one fumarate salt of claim 1, which is crystalline (R)-2-(4-fluorophenyl)-2-(methylamino)cyclohexan-1-one fumarate-2 salt characterized by an X-ray powder diffraction pattern comprising peaks at 12.4, 16.3, and 22.8±0.5°2θ.

142. (canceled)

143. The crystalline (R)-2-(4-fluorophenyl)-2-(methylamino)cyclohexan-1-one fumarate-2 salt of claim 141 characterized by an X-ray powder diffraction pattern comprising peaks at 12.4, 16.3, 17.7, 22.8, and 24.6±0.5 ° 2θ.

144. (canceled)

145. (canceled)

146. (canceled)

147. The crystalline (R)-2-(4-fluorophenyl)-2-(methylamino)cyclohexan-1-one citrate salt of claim 1 which is crystalline (R)-2-(4-fluorophenyl)-2-(methylamino)cyclohexan-1-one citrate-2 salt characterized by an X-ray powder diffraction pattern comprising peaks at 6.6, 14.5, and 17.7±0.5 ° 2θ.

148. (canceled)

149. (canceled)

150. The crystalline (R)-2-(4-fluorophenyl)-2-(methylamino)cyclohexan-1-one citrate-2 salt of claim 147 characterized by an X-ray powder diffraction pattern comprising peaks at 6.6, 14.5, 16.3, 17.7, and 17.9±0.5° 2θ.

151. (canceled)

152. (canceled)

153. (canceled)

154. The crystalline (R)-2-(4-fluorophenyl)-2-(methylamino)cyclohexan-1-one citrate-2 salt of claim 147 characterized by a DSC thermogram having at least one endotherm with a peak at about 173.6 °C.

155. (canceled)

156. The crystalline salt of claim 1, which is crystalline (R)-2-(4-fluorophenyl)-2-(methylamino)cyclohexan-1-one glycolate salt which is characterized by an X-ray powder diffraction pattern comprising peaks at 7.4, 9.6, and 19.2±0.5 ° 2θ.

157. (canceled)

158. The crystalline (R)-2-(4-fluorophenyl)-2-(methylamino)cyclohexan-1-one glycolate salt of claim 156 characterized by an X-ray powder diffraction pattern comprising peaks at 7.4, 9.6, 19.2, 22.2, and 29.8±0.5 ° 2θ.

159. (canceled)

160. (canceled)

161. (canceled)

162. The crystalline (R)-2-(4-fluorophenyl)-2-(methylamino)cyclohexan-1-one glycolate salt of claim 156 characterized by a DSC thermogram having at least one endotherm with a peak at about 118.5 °C.

163. (canceled)

164. The crystalline salt of claim 1, which is crystalline (R)-2-(4-fluorophenyl)-2-(methylamino)cyclohexan-1-one malate salt which is characterized by an X-ray powder diffraction pattern comprising peaks at 13.4, 15.8, and 18.5±0.5 ° 2θ.

165. (canceled)

166. The crystalline (R)-2-(4-fluorophenyl)-2-(methylamino)cyclohexan-1-one malate salt of claim 164 characterized by an X-ray powder diffraction pattern comprising peaks at 13.4, 14.7, 15.8, 18.5, and 19.4±0.5 ° 2θ.

167. (canceled)

168. (canceled)

169. (canceled)

170. The crystalline (R)-2-(4-fluorophenyl)-2-(methylamino)cyclohexan-1-one malate salt of claim 164 characterized by a DSC thermogram having at least one endotherm with a peak at about 196.3 °C or about 126.0 °C.

171. (canceled)

172. (canceled)

173. The crystalline salt of claim 1, which is crystalline (R)-2-(4-fluorophenyl)-2-(methylamino)cyclohexan-1-one hippurate salt which is characterized by an X-ray powder diffraction pattern comprising peaks at 6.2, 8.6, and 12.5±0.5 ° 2θ.

174. (canceled)

175. The crystalline (R)-2-(4-fluorophenyl)-2-(methylamino)cyclohexan-1-one hippurate salt of claim 173 characterized by an X-ray powder diffraction pattern comprising peaks at 6.2, 8.6, 12.5, 18.7, and 20.9±0.5 ° 2θ.

176. (canceled)

177. (canceled)

178. (canceled)

179. The crystalline (R)-2-(4-fluorophenyl)-2-(methylamino)cyclohexan-1-one hippurate salt of claim 173 characterized by a DSC thermogram having at least one endotherm with a peak at about 117.6 °C.

180. (canceled)

181. A solid comprising one or more of the crystalline salts of (R)-2-(4-fluorophenyl)-2-(methylamino)cyclohexan-1-one of claim 1.

182. The solid of claim 181 comprising at least 50% by weight of any one of the crystalline salts of (R)-2-(4-fluorophenyl)-2-(methylamino)cyclohexan-1-one.

183. The solid of claim 181 comprising at least 90% by weight of any one of the crystalline salts of (R)-2-(4-fluorophenyl)-2-(methylamino)cyclohexan-1-one.

184. The solid of claim 181 comprising at least 95% by weight of any one of the crystalline salts of (R)-2-(4-fluorophenyl)-2-(methylamino)cyclohexan-1-one.

185. (canceled)

186. (canceled)

187. A solid comprising a substantially chemically and polymorphically pure form of any one of the crystalline salts of (R)-2-(4-fluorophenyl)-2-(methylamino)cyclohexan-1-one of claim 1.

188. A pharmaceutical composition comprising one or more of the crystalline salts of claim 1.

189. A method of treating a psychiatric disorder comprising administering a therapeutically effective amount of one or more of the crystalline salts of claim 1.
